(19) United States
(12) Patent Application Publication
(10) Pub. No.: US 2014/0256756 A1
(43) Pub. Date: Sep. 11, 2014

(54) PIPERIDINYL-SUBSTITUTED LACTAMS AS GPR119 MODULATORS

(71) Applicant: Array BioPharma Inc., Boulder, CO (US)

(72) Inventors: Thomas Daniel Aicher, Boulder, CO (US); Josef Roland Benscik, Boulder, CO (US); Steven Armen Boyd, Boulder, CO (US); Kevin Ronald Condroski, Boulder, CO (US); Jay Bradford Fell, Boulder, CO (US); John Peter Fischer, Boulder, CO (US); Ronald Jay Hinklin, Boulder, CO (US); Scott Pratt, Boulder, CO (US); Ajay Singh, Boulder, CO (US); Timothy M. Turner, Boulder, CO (US)

(21) Appl. No.: 14/355,804

(22) PCT Filed: Oct. 30, 2012
(86) PCT No.: PCT/US2012/062576

§ 371 Date: May 1, 2014

Related U.S. Application Data
(60) Provisional application No. 61/555,431, filed on Nov. 3, 2011.

(51) Int. Cl. C07D 417/14; C07D 413/14

(52) U.S. Cl. CPC C07D 417/14; C07D 413/14
USPC 514/272; 546/209; 514/326; 544/320; 546/193; 514/318; 546/187; 514/316; 546/194

(57) ABSTRACT

Compounds of Formula (I) and pharmaceutically acceptable salts thereof in which X¹, X², X³, L, R³, R⁴, R⁵, R⁷ and n have the meanings given in the specification, are modulators of GPR119 and are useful in the treatment or prevention of diseases such as, but not limited to, type 2 diabetes, diabetic complications, symptoms of diabetes, metabolic syndrome, obesity, dyslipidemia, and related conditions.

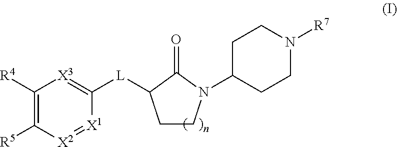

The present invention relates to novel compounds, to pharmaceutical compositions comprising the compounds, to processes for making the compounds, and to the use of the compounds in therapy. More particularly, it relates to certain piperidinyl-substituted lactams which are modulators of GPR119 and are useful in the treatment or prevention of diseases such as, but not limited to, type 2 diabetes, diabetic complications, symptoms of diabetes, metabolic syndrome, obesity, dyslipidemia, and related conditions. In addition, the compounds are useful in decreasing food intake, decreasing weight gain, and increasing satiety in mammals.

Diabetes is diagnosed by elevated fasting plasma glucose levels ≧126 mg/dL or by plasma glucose levels after an oral glucose tolerance test ≧200 mg/dL. Diabetes is associated with the classic symptoms of polydipsia, polyphagia and polyuria (The Expert Committee on the Diagnosis and Classification of Diabetes Mellitus, Diabetes Care, 1998, 21, S5-19). Of the two major forms of diabetes, insulin dependent diabetes mellitus (Type I) accounts for 5-10% of the diabetic population. Type I diabetes is characterized by near total beta cell loss in the pancreas and little or no circulating insulin. Non-insulin dependent diabetes mellitus (Type 2 diabetes) is the more common form of diabetes. Type 2 diabetes is a chronic metabolic disease that develops from a combination of insulin resistance in the muscle, fat, and liver and from partial beta cell loss in the pancreas. The disease progresses with the inability of the pancreas to secrete sufficient insulin to overcome such resistance. Uncontrolled type 2 diabetes is associated with an increased risk of heart disease, stroke, neuropathy, retinopathy and nephropathy among other diseases.

Obesity is a medical condition characterized by high levels of adipose tissue in the body. Body mass index is calculated by dividing weight by height squared (BMI=kg/m²), where a person with a BMI of ≧30 is considered obese and medical intervention is recommended (For the Clinical Efficacy Assessment Subcommittee of the American College of Physicians. Pharmacological and surgical management of obesity in primary care: a clinical practice guideline from the American College of Physicians. Ann Intern Med, 2005, 142, 525-531). The main causes of obesity are increased calorie intake accompanied with a lack of physical activity and genetic predisposition. Obesity leads to an increased risk of many diseases including, but not limited to, diabetes, heart disease, stroke, dementia, cancer, and osteoarthritis.

Metabolic syndrome is present when a group of risk factors are found in a mammal (Grundy, S. M.; Brewer, H. B. Jr.; et al., Circulation, 2004, 109, 433-438). Abdominal obesity, dyslipidemia, high blood pressure and insulin resistance predominate in this disease. Similar to obesity, metabolic syndrome results from increased calorie intake, physical inactivity, and aging. Of major concern is that this condition can lead to coronary artery disease and type 2 diabetes.

Clinically there are a number of treatments currently being used to lower blood glucose in type 2 diabetic patients. Metformin (De Fronzo, R. A.; Goodman, A. M., N. Engl. J. Med., 1995, 333, 541-549) and the PPAR agonists (Wilson, T. M., et al., J. Med. Chem., 1996, 39, 665-668) partially ameliorate insulin resistance by improving glucose utilization in cells. Treatment with sulfonylureas (Blickle, J. F., Diabetes Metab. 2006 32, 113-120) has been shown to promote insulin secretion by affecting the pancreatic KATP channel; however, the increase in insulin is not glucose dependent and such treatment can lead to hypoglycemia. The recently approved DPP4 inhibitors and GLP-1 mimetics promote insulin secretion by the beta cell through an incretin mechanism, and administration of these agents causes insulin release in a glucose dependent manner (Vahl, T. P., D'Alessio, D. A., Expert Opinion on Invest. Drugs, 2004, 13, 177-188). However, even with these newer treatments, it is difficult to achieve precise control of blood glucose levels in type 2 diabetic patients in accordance with the guidelines recommended by the American Diabetes Association.

GPR119 is a Gs-coupled receptor that is predominately expressed in the pancreatic beta cells and in the enteroendocrine K and L cells of the GI tract. In the gut, this receptor is activated by endogenous lipid-derived ligands such as oleoylethanolamide (Lauffer, L. M., et al., Diabetes, 2009, 58, 1058-1066). Upon activation of GPR119 by an agonist, the enteroendocrine cells release the gut hormones glucagon like peptide 1 (GLP-1), glucose-dependent insulinotropic peptide (GIP), and peptide YY (PYY) among others. GLP-1 and GIP have multiple mechanisms of action that are important for controlling blood glucose levels (Parker, H. E., et al., Diabetologia, 2009, 52, 289-298). One action of these hormones is to bind to GPCRs on the surface of beta cells leading to a rise in intracellular cAMP levels. This rise results in a glucose dependent release of insulin by the pancreas (Drucker, D. J. J. Clin. Investigation, 2007, 117, 24-32; Winzell, M. S., Pharmacol. and Therap. 2007, 116, 437-448). In addition, GLP-1 and GIP have been shown to increase beta cell proliferation and decrease the rate of apoptosis in vivo in animal models of diabetes and in vitro with human beta cells (Farilla, L.; et al., Endocrinology, 2002, 143, 4397-4408; Farilla, L.; et al., Endocrinology, 2003, 144 5149-5158; and Hughes, T. E., Current Opin. Chem. Biol., 2009, 13, 1-6). Current GLP-1 mechanism based therapies, such as sitagliptin and exenatide, are clinically validated to improve glucose control in type 2 diabetic patients.

GPR119 receptors are also expressed directly on the pancreatic beta cells. A GPR119 agonist can bind to the pancreatic GPR119 receptor and cause a rise in cellular cAMP levels consistent with a Gs-coupled GPCR signaling mechanism. The increased cAMP then leads to a release of insulin in a glucose dependent manner. The ability of GPR119 agonists to enhance glucose-dependent insulin release by direct action on the pancreas has been demonstrated in vitro and in vivo (Chu Z., et al., Endocrinology 2007, 148:2601-2609). This dual mechanism of action of the release of incretin hormones in the gut and binding directly to receptors on the pancreas may offer an advantage for GPR119 agonists over current therapies for treating diabetes.

GPR119 agonists, by increasing the release of PYY, may also be of benefit in treating many of comorbidities associated with diabetes and to treat these diseases in the absence of diabetes. Administration of PYY₃₋₃₆ has been reported to reduce food intake in animals (Batterham, R. L., et al., Nature, 2002, 418, 650-654), increase satiety and decrease food intake in humans (Batterham, R. L., et al., Nature, 2002, 418, 650-654), increase resting body metabolism (Sloth B., et al., Am. J. Physiol. Endocrinol. Metab., 2007, 292, E1062-1068 and Guo, Y., et al., Obesity, 2006, 14, 1562-1570), increase fat oxidation (Adams, S. H., et al., J. Nutr., 2006, 136, 195-201 and van den Hoek, A. M., et al., Diabetes, 2004, 53, 1949-1952), increase thyroid hormone activity, and increase adiponectin levels. PYY release caused by GPR119 agonists can therefore be beneficial in treating the metabolic syndrome and obesity.

Several classes of small molecule GPR119 agonists are known (Fyfe, M. T. E. et al., Expert Opin. Drug. Discov., 2008, 3(4), 403-413; Jones, R. M., et al., Expert Opin. Ther. Patents, 2009, 19(10), 1339-1359).

There remains, however, a need for compounds and methods for the treatment or prevention of diabetes, dyslipidemia, diabetic complications, and obesity.

### SUMMARY OF THE INVENTION

It has now been found that novel piperidinyl-substituted lactams are modulators of GPR119 and may be useful for treating type 2 diabetes, diabetic complications, metabolic syndrome, obesity, dyslipidemia, and related conditions.

Accordingly, in one aspect of the present invention there is provided compounds having the general Formula I

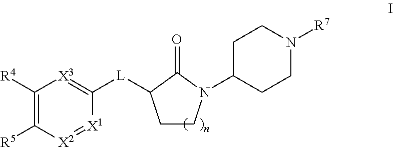

and pharmaceutically acceptable salts thereof, wherein X¹, X², X³, L, R⁴, R⁵, R⁷ and n are as defined herein.

In another aspect of the invention, there are provided pharmaceutical compositions comprising a compound of Formula I and a pharmaceutically acceptable carrier, diluent or excipient.

In another aspect of the invention, there is provided a method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I or a pharmaceutically acceptable salt thereof. In one embodiment, the disease is type 2 diabetes. In one embodiment, the method comprises administering a compound of Formula I in combination with one or more additional drugs. In one embodiment, the additional drug is a biguanide. In one embodiment, the additional drug is a DPP4 inhibitor.

In another aspect of the invention, there is provided the use of a compound of Formula I in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

In another aspect of the invention, there is provided compounds of Formula I or pharmaceutically acceptable salts thereof, for use in therapy.

In another aspect of the invention, there is provided compounds of Formula I or pharmaceutically acceptable salts thereof, for use in treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis, diabetic complications, metabolic syndrome (including hyperglycemia, impaired glucose tolerance, and insulin resistance), obesity, dyslipidemia, dyslipoproteinemia.

In another aspect of the invention, there is provided compounds of Formula I or pharmaceutically acceptable salts thereof, for use in treating type 2 diabetes.

Another aspect of the invention provides intermediates for preparing compounds of Formula I. In one embodiment, certain compounds of Formula I may be used as intermediates for the preparation of other compounds of Formula I.

Another aspect of the invention includes processes for preparing, methods of separation, and methods of purification of the compounds described herein.

### DETAILED DESCRIPTION OF THE INVENTION

One embodiment of this invention provides compounds of the general Formula I

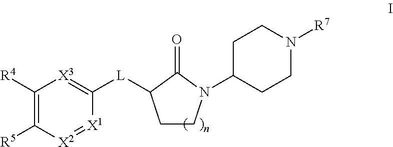

and pharmaceutically acceptable salts thereof, wherein:

L is O, NR^{x} or CH₂;

R^{x} is H or (1-3C)alkyl;

X¹ is N or CR¹, X² is N or CR², and X³ is N or CR³, wherein only one of X¹ and X² may be N;

R¹, R², R³ and R⁴ are independently selected from H, halogen, CF₃, (1-6C)alkyl, CN and (1-6C)alkoxy;

R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, Br, tetrazolyl optionally substituted with (1-3C)alkyl, or oxadiazolyl optionally substituted with (1-3C)alkyl;

R′, R″ and R′″ are independently H or (1-4C)alkyl;

R⁷ is selected from

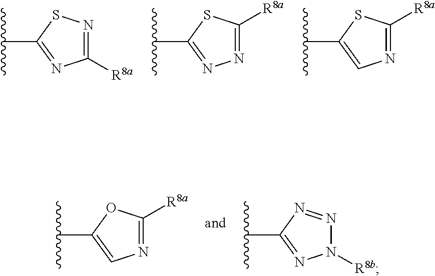

R^{8a} is selected from (1-6C)alkyl, fluoro(1-6C)alkyl, difluoro(1-6C)alkyl, trifluoro(1-6C)alkyl, (1-3C alkoxy)(1-6C)alkyl, dihydroxy(2-6C)alkyl, Br, Cyc¹, Ar¹, —OAr¹, hetCyc¹, hetAr¹ and —OhetAr¹;

R^{8b} is (1-6C)alkyl;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having 1-2 ring heteroatoms and optionally substituted with one or more groups independently selected from (1-4C)alkyl;

hetAr¹ is a 5-6-membered heteroaryl having 1-2 ring heteroatoms and optionally substituted with one or more substituents independently selected from (1-4C)alkyl, halogen, CF₃ and (1-4C)alkoxy; and

n is 1, 2 or 3.

In one embodiment of Formula I, n is 1.

In one embodiment of Formula I, n is 2.

In one embodiment of Formula I, n is 3.

In one embodiment of Formula I, L is O.

In one embodiment of Formula I, L is NR^{x}.

In one embodiment, L is NH.

In one embodiment, L is N(1-3C)alkyl. Particular examples include NCH₃ and NCH₂CH₃.

In one embodiment, L is CH₂.

In one embodiment, X¹ is CR¹ and R¹ is H, F, Cl, CN, Me or CF₃.

In one embodiment, R¹ is H, F or Cl.

In one embodiment, R¹ is H.

In one embodiment, R¹ is F.

In one embodiment, R¹ is Cl.

In one embodiment, X² is CR² and R² is H, F, Me or CF₃.

In one embodiment, R² is H.

In one embodiment, X³ is CR³ and R³ is H, F, Cl or CF₃.

In one embodiment, R³ is H.

In one embodiment, R⁴ is H, Me, F, or Cl.

In one embodiment, R⁴ is H.

In one embodiment, R⁴ is Me.

In one embodiment, R⁴ is F.

In one embodiment, R⁴ is Cl.

In one embodiment of Formula I, the residue:

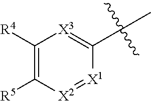

of Formula I, wherein the wavy line represents the point of attachment of the residue in Formula I, is selected from a residue wherein X¹ is CR¹, X² is CR², and X³ is CR³, such that the residue can be represented as:

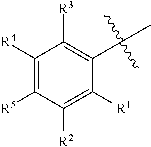

wherein R¹, R², R³, R⁴ and R⁵ are as defined for Formula I. In one embodiment of Formula I, R¹, R², R³ and R⁴ are independently selected from H, (1-6C)alkyl, CF₃, CN and halogen. In one embodiment, R¹, R², R³ and R⁴ are independently selected from H, F, Cl, CF₃, CN, methyl, ethyl, and propyl. In one embodiment, R¹ is H, F, Cl, CF₃, CN or Me, R² is H, F or Cl, R³ is H, and R⁴ is H, Me, F, or Cl.

In one embodiment of Formula I, the residue:

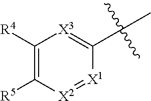

of Formula I, wherein the wavy line represents the point of attachment of the residue to “L” in Formula I, is selected from a residue wherein X¹ is N, X² is CR², and X³ is CR³, such that the residue can be represented as:

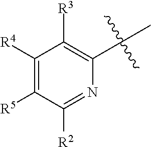

wherein R², R³, R⁴ and R⁵ are as defined for Formula I. In one embodiment, R², R³ and R⁴ are independently selected from H, halogen, CF₃ and (1-6C)alkyl. In one embodiment, R², R³ and R⁴ are independently selected from H, F, Cl, CF₃, methyl, ethyl, propyl, and isopropyl. In one embodiment, R², R³ and R⁴ are independently selected from H, F, Cl and Me. In one embodiment, R², R³ and R⁴ are each H.

In one embodiment of Formula I, the residue:

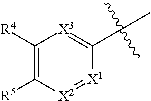

of Formula I, wherein the wavy line represents the point of attachment of the residue to “L” in Formula I, is selected from a residue wherein X¹ is CR¹, X² is N, and X³ is CR³, such that the residue can be represented as:

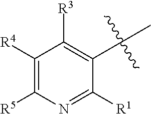

wherein R¹, R³, R⁴ and R⁵ are as defined for Formula I. In one embodiment, R¹, R³ and R⁴ are independently selected from H, halogen, CF₃ and (1-6C)alkyl. In one embodiment, R¹, R³ and R⁴ are independently selected from H, F, Cl, CF₃, methyl, ethyl, propyl and isopropyl. In one embodiment, R¹, R³ and R⁴ are independently selected from H, F, Cl and Me. In one embodiment, each of R¹, R³ and R⁴ is H.

In one embodiment of Formula I, the residue:

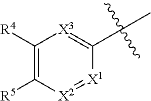

of Formula I, wherein the wavy line represents the point of attachment of the residue to “L” in Formula I, is selected from a residue wherein X¹ is N, X² is CR², and X³ is N, such that the residue can be represented as:

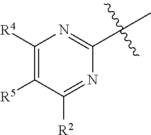

wherein R², R⁴ and R⁵ are as defined for Formula I. In one embodiment, R² and R⁴ are independently selected from H, halogen, CF₃ and (1-6C)alkyl. In one embodiment, R² and R⁴ are independently selected from H, F, Cl, CF₃, methyl, ethyl, propyl and isopropyl. In one embodiment, R² and R⁴ are independently selected from H, F, Cl and Me. In one embodiment, each of R² and R⁴ is H.

In one embodiment of Formula I, R⁵ is selected from (1-3C alkyl)sulfonyl, (3-6C cycloalkyl)sulfonyl, (cyclopropylmethyl)sulfonyl and phenylsulfonyl.

In one embodiment, R⁵ is (1-3C alkyl)sulfonyl. Examples include CH₃SO₂—, CH₃CH₂SO₂—, CH₃CH₂CH₂SO₂— and (CH₃)₂CHSO₂—. In one embodiment, R⁵ is CH₃SO₂—. In one embodiment, R⁵ is CH₃CH₂SO₂—. In one embodiment, R⁵ is (CH₃)₂CHSO₂—.

In one embodiment, R⁵ is (3-6C cycloalkyl)sulfonyl. An example is (cyclopropyl)SO₂—.

In one embodiment, R⁵ is (cyclopropylmethyl)sulfonyl which can be represented by the structure:

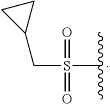 [SMILES: *S(=O)(=O)CC1CC1]

In one embodiment, R⁵ is phenylsulfonyl.

In one embodiment, R⁵ is CH₃SO₂—, CH₃CH₂SO₂—, CH₃CH₂CH₂SO₂—, (CH₃)₂CHSO₂—, (cyclopropyl)SO₂—, (cyclopropylmethyl)sulfonyl or phenylsulfonyl.

In one embodiment, R⁵ is selected from di(1-3C alkyl)NSO₂—, (1-3C alkyl)S—, HOCH₂CH₂NHC(═O)— and R′R″NCH₂CH₂NR′″C(═O)—.

In one embodiment, R⁵ is di(1-3C alkyl)NSO₂—. An example is (CH₃)₂NSO₂—.

In one embodiment, R⁵ is (1-3C alkyl)S—. An example is CH₃S—.

In one embodiment, R⁵ is HOCH₂CH₂NHC(═O)—.

In one embodiment, R⁵ is R′R″NCH₂CH₂NR′″C(═O)—. In one embodiment, R′ and R″ are hydrogen or (1-4C)alkyl and R′″ is hydrogen. In one embodiment, R′ and R″ are hydrogen or (1-4C)alkyl and R′″ is (1-4C)alkyl. Examples of R⁵ include H₂NCH₂CH₂NCH₃C(═O)—, (CH₃)₂NCH₂CH₂NHC(═O)— and (CH₃)NCH₂CH₂N(CH₃)C(═O)—.

In one embodiment, R⁵ is CN or Br.

In one embodiment, R⁵ is CN.

In one embodiment, R⁵ is Br.

In one embodiment, R⁵ is tetrazolyl or oxadiazolyl, each of which is optionally substituted with (1-3C)alkyl.

In one embodiment, R⁵ is tetrazolyl optionally substituted with (1-3C)alkyl. In one embodiment, R⁵ is tetrazolyl optionally substituted with methyl. Particular examples of R⁵ include groups having the structures:

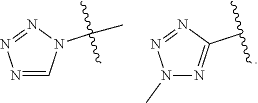

In one embodiment, R⁵ is oxadiazolyl optionally substituted with (1-3C)alkyl. An example includes the structure:

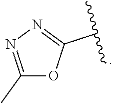 [SMILES: *c1nnc(C)o1]

Particular examples of the group having the structure

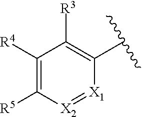

include the following structures:

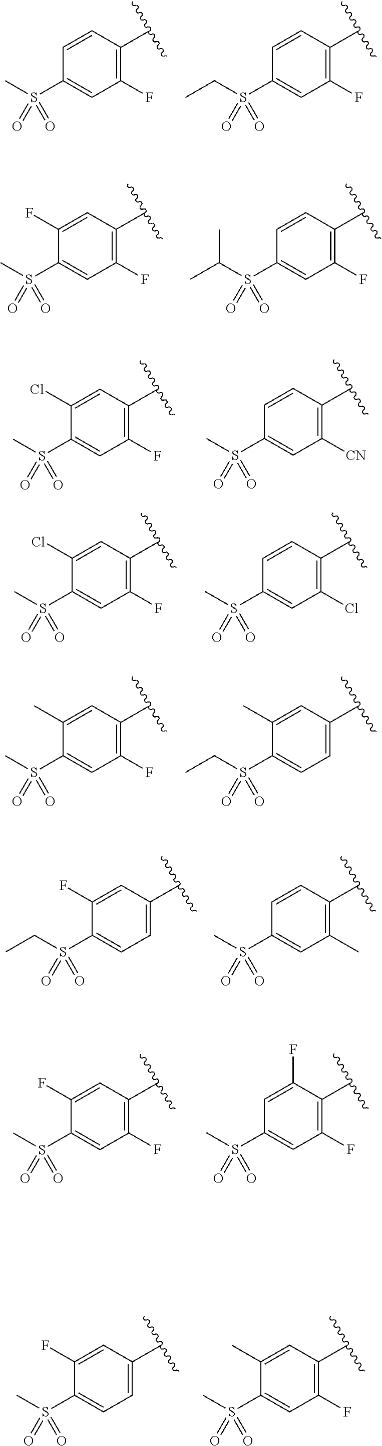

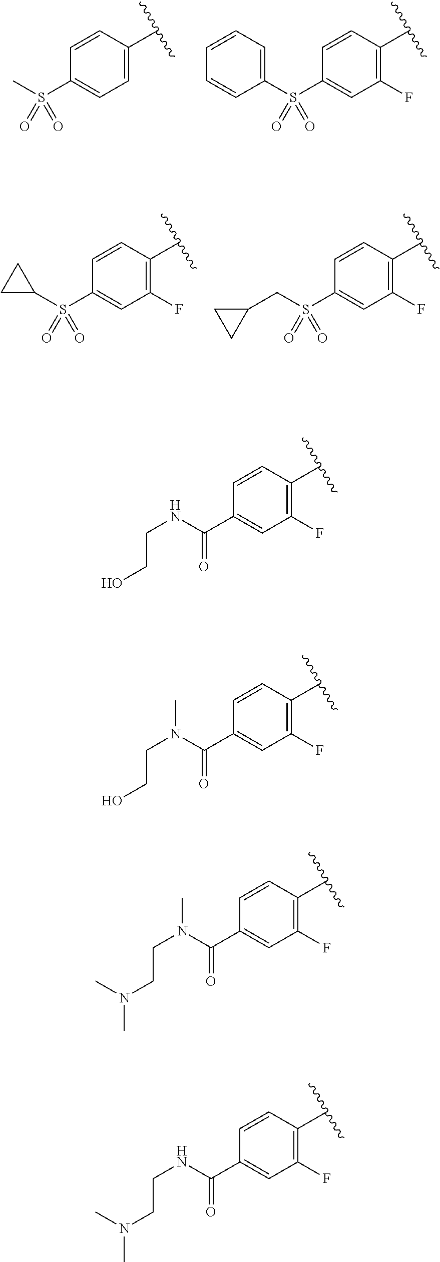

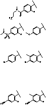

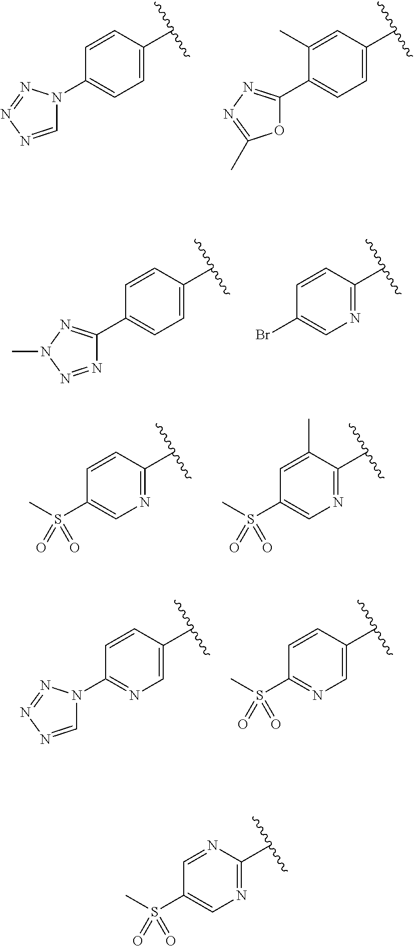

In one embodiment, the group having the structure

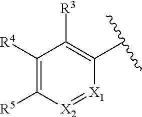

is selected from the structures:

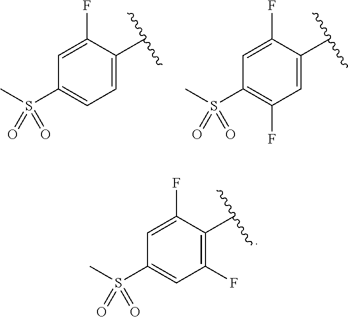 [SMILES: *c1ccc(S(C)(=O)=O)cc1F ; *c1cc(F)c(S(C)(=O)=O)cc1F ; *c1c(F)cc(S(C)(=O)=O)cc1F]

In one embodiment of Formula I, R⁷ is selected from the structures:

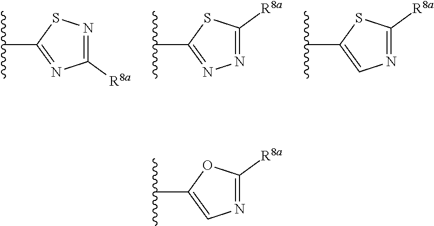

wherein R^{8a} is as defined for Formula I.

In one embodiment of Formula I, R^{8a} is selected from (1-6C)alkyl, fluoro(1-6C)alkyl, difluoro (1-6C)alkyl, trifluoro (1-6C)alkyl, (1-3C alkoxy)(1-6C)alkyl, Cyc¹, and -dihydroxy(2-6C)alkyl.

In one embodiment of Formula I, R^{8a} is (1-6C)alkyl. In one embodiment, R^{8a} is methyl, ethyl, propyl, sec-propyl, butyl, isobutyl or tert-butyl. In one embodiment, R^{8a} is ethyl, isopropyl, sec-butyl or tert-butyl. In one embodiment, R^{8a} is isopropyl.

In one embodiment of Formula I, R^{8a} is fluoro(1-6C)alkyl. In one embodiment, R^{8a} is 2-fluoropropyl.

In one embodiment of Formula I, R^{8a} is difluoro(1-6C)alkyl. In one embodiment, R^{8a} is difluoromethyl, 1,1-difluoroethyl or 1,1-difluoropropyl.

In one embodiment of Formula I, R^{8a} is trifluoro(1-6C)alkyl. In one embodiment, R^{8a} is trifluoromethyl or 1,1-dimethyl-2,2-difluoroethyl.

In one embodiment of Formula I, R^{8a} is (1-3C alkoxy)(1-6C)alkyl. In one embodiment, R^{8a} is 2-methoxyprop-2-yl.

In one embodiment of Formula I, R^{8a} is Cyc¹. In one embodiment, R^{8a} is cyclopropyl, cyclobutyl or cyclopentyl optionally substituted with CF₃. In one embodiment, R^{8a} is cyclopropyl, 1-(trifluoromethyl)cyclopropyl, cyclobutyl or cyclopentyl.

In one embodiment of Formula I, R^{8a} is dihydroxy(2-6C)alkyl. In one embodiment, R^{8a} is —CH(OH)CH₂OH.

In one embodiment of Formula I, R^{8a} is Br.

In one embodiment of Formula I, R^{8a} is selected from Ar¹, —OAr¹, hetCyc¹, hetAr¹, and —OhetAr¹.

In one embodiment of Formula I, R^{8a} is Ar¹. In one embodiment, Ar¹ is phenyl optionally substituted with one or more groups independently selected from F, Cl, CF₃, methyl, ethyl and methoxy. In one embodiment, R^{8a} is phenyl.

In one embodiment of Formula I, R^{8a} is —OAr¹. In one embodiment, Ar¹ is phenyl optionally substituted with one or more groups independently selected from F, Cl, CF₃, methyl, ethyl and methoxy. In one embodiment, R^{8a} is phenoxy.

In one embodiment of Formula I, R^{8a} is hetCyc¹. In one embodiment, R^{8a} is tetrahydro-2H-pyranyl or pyrrolidinyl optionally substituted with one or more groups independently selected from F, Cl, CF₃, methyl, ethyl and methoxy. In one embodiment, R^{8a} is tetrahydro-2H-pyranyl optionally substituted with methyl. In one embodiment R^{8a} is selected from the structures:

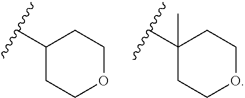

In one embodiment of Formula I, R^{8a} is hetAr¹. In one embodiment, hetAr¹ is a 5-6 membered heteroaryl having 1-2 ring heteroatoms independently selected from N and O and optionally substituted with one or more substituents independently selected from (1-4C)alkyl, halogen, CF₃, and (1-4C)alkoxy. In one embodiment, hetAr¹ is a pyrazolyl, oxazolyl, or pyridyl optionally substituted with one or more substituents independently selected from (1-4C)alkyl, halogen, CF₃, and (1-4C)alkoxy. In one embodiment, R^{8a} is pyrazolyl, oxazolyl, or pyridyl optionally substituted with one or more substituents independently selected from F, Cl, CF₃, methyl, ethyl and methoxy. In one embodiment, R^{8a} is selected from the structures:

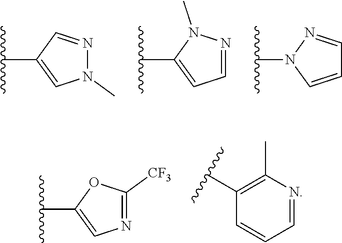

In one embodiment of Formula I, R^{8a} is —O-hetAr¹. In one embodiment, hetAr¹ is a 5-6 membered heteroaryl having 1-2 ring heteroatoms independently selected from N and O and optionally substituted with one or more substituents independently selected from (1-4C)alkyl, halogen, CF₃ and (1-4C)alkoxy. In one embodiment, hetAr¹ is pyrazolyl or pyridyl optionally substituted with one or more substituents independently selected from (1-4C)alkyl, halogen, CF₃ and (1-4C)alkoxy. In one embodiment, R^{8a} is pyrazolyl or pyridyl optionally substituted with one or more substituents independently selected from (1-4C)alkyl. In one embodiment, R^{8a} is selected from the structures:

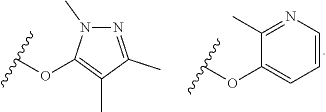

In one embodiment of Formula I, R⁷ has the structure:

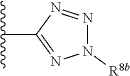

where R^{8b} is (1-6C)alkyl. In one embodiment, R⁷ has the structure:

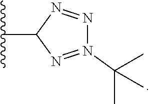

Particular examples of the group R⁷ include the structures:

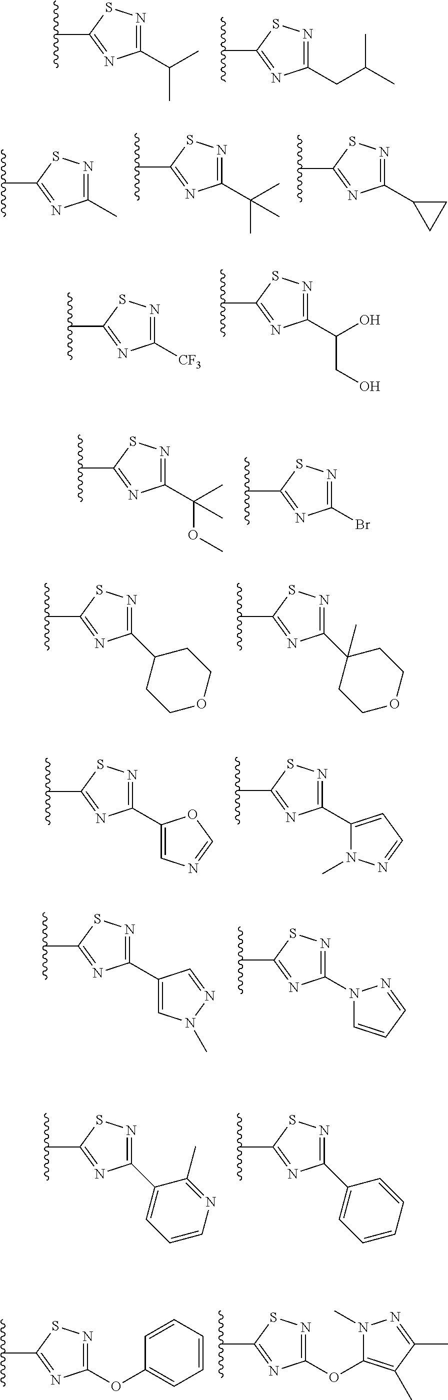

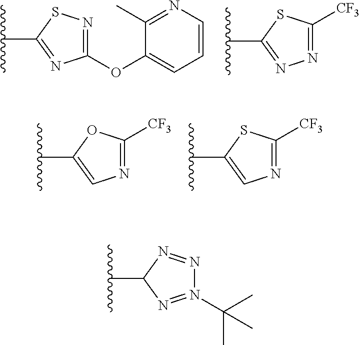

In embodiment, compounds of Formula I include compounds of Formula IA and pharmaceutically acceptable salts thereof, wherein:

L is O or NR^{x};

R^{x} is H or (1-3C)alkyl;

X¹ is CR¹, X² is CR² and X³ is CR³;

R¹, R², R³ and R⁴ are independently selected from H, halogen, CN and (1-6C)alkyl;

R⁵ is (1-3C alkyl)sulfonyl, (3-6C cycloalkyl)sulfonyl or (cyclopropylmethyl)sulfonyl;

R⁷ is

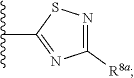

R^{8a} is selected from (1-6C)alkyl, fluoro(1-6C)alkyl, difluoro(1-6C)alkyl, trifluoro(1-6C)alkyl, Cyc¹, Ar¹, —OAr¹, hetCyc¹, hetAr¹, —OhetAr¹, —CH(OH)CH₂OH, —C(CH₃)₂(OMe) and Br;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having 1-2 ring heteroatoms and optionally substituted with one or more groups independently selected from (1-4C)alkyl;

hetAr¹ is a 5-6-membered heteroaryl having 1-2 ring heteroatoms and optionally substituted with one or more substituents independently selected from (1-4C)alkyl, halogen, CF₃, and (1-4C)alkoxy; and

n is 1, 2 or 3.

In one embodiment of Formula IA, L is O; and R¹, R², R³, R⁴, R⁵, R^{8a}, Cyc¹, Ar¹, hetCyc¹, hetAr¹ and n are as defined for Formula IA.

In one embodiment of Formula IA, L is O; R^{8a} is (1-6C)alkyl, fluoro(1-6C)alkyl, difluoro(1-6C)alkyl or trifluoro(1-6C)alkyl; and R¹, R², R³, R⁴, R⁵ and n are as defined for Formula IA.

In one embodiment of Formula IA, L is O; R^{8a} is (1-6C)alkyl, fluoro(1-6C)alkyl, difluoro(1-6C)alkyl or trifluoro(1-6C)alkyl; R¹, R², R³ and R⁴ are independently H, halogen or (1-6C)alkyl; and R⁵ and n are as defined for Formula IA.

In one embodiment of Formula IA, L is NR^{x}; and R^{x}, R¹, R², R³, R⁴, R⁵, R^{8a}, Cyc¹, Ar¹, hetCyc¹, hetAr¹ and n are as defined for Formula IA.

In one embodiment of Formula IA, L is NR^{x}; R^{8a} is (1-6C)alkyl, fluoro(1-6C)alkyl, difluoro(1-6C)alkyl or trifluoro(1-6C)alkyl; and R^{x}, R¹, R², R³, R⁴, R⁵, and n are as defined for Formula IA.

In one embodiment of Formula IA, L is NR^{x}; R^{8a} is (1-6C)alkyl, fluoro(1-6C)alkyl, difluoro(1-6C)alkyl or trifluoro(1-6C)alkyl; R¹, R², R³ and R⁴ are independently H, halogen or (1-6C)alkyl; and R^{x}, R⁵, and n are as defined for Formula IA.

In embodiment, compounds of Formula I include compounds of Formula IB and pharmaceutically acceptable salts thereof, wherein:

L is O or NR^{x};

R^{x} is H or (1-3C)alkyl;

X¹ is N, X² is CR² and X³ is CR³;

R², R³ and R⁴ are independently selected from H, halogen, CF₃, (1-6C)alkyl and (1-6C)alkoxy;

R⁵ is (1-3C alkyl)sulfonyl, (3-6C cycloalkyl)sulfonyl or (cyclopropylmethyl)sulfonyl;

R⁷ is

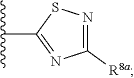

R^{8a} is selected from (1-6C)alkyl, fluoro(1-6C)alkyl, difluoro(1-6C)alkyl, trifluoro(1-6C)alkyl, Cyc¹, Ar¹, —OAr¹, hetCyc¹, hetAr¹, —OhetAr¹, —CH(OH)CH₂OH, —C(CH₃)₂(OMe) and Br;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having 1-2 ring heteroatoms and optionally substituted with one or more groups independently selected from (1-4C)alkyl;

hetAr¹ is a 5-6-membered heteroaryl having 1-2 ring heteroatoms and optionally substituted with one or more substituents independently selected from (1-4C)alkyl, halogen, CF₃, and (1-4C)alkoxy; and

n is 1, 2 or 3.

In one embodiment of Formula IB, L is O; and R², R³, R⁴, R⁵, R^{8a}, Cyc¹, Ar¹, hetCyc¹, hetAr¹ and n are as defined for Formula IB.

In one embodiment of Formula IB, L is O; R^{8a} is (1-6C)alkyl, fluoro(1-6C)alkyl, difluoro(1-6C)alkyl or trifluoro(1-6C)alkyl; and R², R³, R⁴, R⁵ and n are as defined for Formula IB.

In one embodiment of Formula IB, L is O; R^{8a} is (1-6C)alkyl, fluoro(1-6C)alkyl, difluoro(1-6C)alkyl or trifluoro(1-6C)alkyl; R², R³, and R⁴ are independently H, halogen or (1-6C)alkyl; and R⁵ and n are as defined for Formula IB.

In one embodiment of Formula IB, L is NR^{x}; and R^{x}, R², R³, R⁴, R⁵, R^{8a}, Cyc¹, Ar¹, hetCyc¹, hetAr¹ and n are as defined for Formula IB.

In one embodiment of Formula IB, L is NR^{x}; R^{8a} is (1-6C)alkyl, fluoro(1-6C)alkyl, difluoro(1-6C)alkyl or trifluoro(1-6C)alkyl; and R^{x}, R², R³, R⁴, R⁵, and n are as defined for Formula IB.

In one embodiment of Formula IB, L is NR^{x}; R^{8a} is (1-6C)alkyl, fluoro(1-6C)alkyl, difluoro(1-6C)alkyl or trifluoro(1-6C)alkyl; R², R³ and R⁴ are independently H, halogen or (1-6C)alkyl; and R^{x}, R⁵ and n are as defined for Formula IB.

In embodiment, compounds of Formula I include compounds of Formula IC and pharmaceutically acceptable salts thereof, wherein:

L is O or NR^{x};

R^{x} is H or (1-3C)alkyl;

X¹ is N, X² is CR² and X³ is N;

R² and R⁴ are independently selected from H and halogen;

R⁵ is (1-3C alkyl)sulfonyl, (3-6C cycloalkyl)sulfonyl or (cyclopropylmethyl)sulfonyl;

R⁷ is

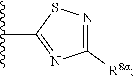

R^{8a} is selected from (1-6C)alkyl, fluoro(1-6C)alkyl, difluoro(1-6C)alkyl, trifluoro (1-6C)alkyl, Cyc¹, Ar¹, —OAr¹, hetCyc¹, hetAr¹, —OhetAr¹, —CH(OH)CH₂OH, —C(CH₃)₂(OMe), —N(1-6C alkyl)₂ and Br;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having 1-2 ring heteroatoms and optionally substituted with one or more groups independently selected from (1-4C)alkyl;

hetAr¹ is a 5-6-membered heteroaryl having 1-2 ring heteroatoms and optionally substituted with one or more substituents independently selected from (1-4C)alkyl, halogen, CF₃ and (1-4C)alkoxy; and

n is 1, 2 or 3.

In one embodiment of Formula IC, L is O; and R², R⁴, R⁵, R^{8a}, Cyc¹, Ar¹, hetCyc¹, hetAr¹ and n are as defined for Formula IC.

In one embodiment of Formula IC, L is O; R^{8a} is (1-6C)alkyl, fluoro(1-6C)alkyl, difluoro(1-6C)alkyl or trifluoro(1-6C)alkyl; and R², R⁴, R⁵ and n are as defined for Formula IC.

In one embodiment of Formula IC, L is O; R^{8a} is (1-6C)alkyl, fluoro(1-6C)alkyl, difluoro(1-6C)alkyl or trifluoro(1-6C)alkyl; R² and R⁴ are independently H, halogen and (1-6C)alkyl; and R⁵ and n are as defined for Formula IC.

In one embodiment of Formula IC, L is NR^{x}; and R^{x}, R², R⁴, R⁵, R^{8a}, Cyc¹, Ar¹, hetCyc¹, hetAr¹ and n are as defined for Formula IC.

In one embodiment of Formula IC, L is NR^{x}; R^{8a} is (1-6C)alkyl, fluoro(1-6C)alkyl, difluoro(1-6C)alkyl or trifluoro(1-6C)alkyl; and R^{x}, R², R⁴, R⁵ and n are as defined for Formula IC.

In one embodiment of Formula IC, L is NR^{x}; R^{8a} is (1-6C)alkyl, fluoro(1-6C)alkyl, difluoro(1-6C)alkyl or trifluoro(1-6C)alkyl; R² and R⁴ are independently H, halogen and (1-6C)alkyl; and R^{x}, R⁵ and n are as defined for Formula IC.

In embodiment, compounds of Formula I include compounds of Formula ID and pharmaceutically acceptable salts thereof, wherein:

L is O or NR^{x};

R^{x} is H or (1-3C)alkyl;

X¹ is CR¹, X² is N and X³ is CR³;

R¹, R³ and R⁴ are independently selected from H and halogen;

R⁵ is (1-3C alkyl)sulfonyl, (3-6C cycloalkyl)sulfonyl or (cyclopropylmethyl)sulfonyl;

R⁷ is

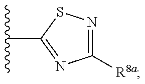

R^{8a} is selected from (1-6C)alkyl, fluoro(1-6C)alkyl, difluoro(1-6C)alkyl, trifluoro (1-6C)alkyl, Cyc¹, Ar¹, —OAr¹, hetCyc¹, hetAr¹, —OhetAr¹, —CH(OH)CH₂OH, —C(CH₃)₂(OMe), —N(1-6C alkyl)₂ and Br;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having 1-2 ring heteroatoms and optionally substituted with one or more groups independently selected from (1-4C)alkyl;

hetAr¹ is a 5-6-membered heteroaryl having 1-2 ring heteroatoms and optionally substituted with one or more substituents independently selected from (1-4C)alkyl, halogen, CF₃, and (1-4C)alkoxy; and

n is 1, 2 or 3.

In one embodiment of Formula ID, L is O; and R¹, R³, R⁴, R⁵, R^{8a}, Cyc¹, Ar¹, hetCyc¹, hetAr¹ and n are as defined for Formula ID.

In one embodiment of Formula ID; L is O; R^{8a} is (1-6C)alkyl, fluoro(1-6C)alkyl, difluoro(1-6C)alkyl or trifluoro(1-6C)alkyl; and R¹, R³, R⁴, R⁵ and n are as defined for Formula ID.

In one embodiment of Formula ID; L is O; R^{8a} is (1-6C)alkyl, fluoro(1-6C)alkyl, difluoro(1-6C)alkyl or trifluoro(1-6C)alkyl; R¹, R³ and R⁴ are independently H, halogen and (1-6C)alkyl; and R⁵ and n are as defined for Formula ID.

In one embodiment of Formula ID, L is NR^{x}; and R^{x}, R¹, R³, R⁴, R⁵, R^{8a}, Cyc¹, Ar¹, hetCyc¹, hetAr¹ and n are as defined for Formula ID.

In one embodiment of Formula ID, L is NR^{x}; R^{8a} is (1-6C)alkyl, fluoro(1-6C)alkyl, difluoro(1-6C)alkyl or trifluoro(1-6C)alkyl; and R^{x}, R¹, R³, R⁴, R⁵, and n are as defined for Formula ID.

In one embodiment of Formula ID, L is NR^{x}; R^{8a} is (1-6C)alkyl, fluoro(1-6C)alkyl, difluoro(1-6C)alkyl or trifluoro(1-6C)alkyl; R¹, R³ and R⁴ are independently H, halogen and (1-6C)alkyl; and R^{x}, R⁵, and n are as defined for Formula ID.

In embodiment, compounds of Formula I include compounds of Formula IE and pharmaceutically acceptable salts thereof, wherein:

L is CH₂;

X¹ is CR¹, X² is CR² and X³ is CR³;

R¹, R², R³ and R⁴ are independently selected from H, halogen, CN and (1-6C)alkyl;

R⁵ is (1-3C alkyl)sulfonyl, (3-6C cycloalkyl)sulfonyl or (cyclopropylmethyl)sulfonyl;

R⁷ is

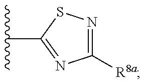

R^{8a} is selected from (1-6C)alkyl, fluoro(1-6C)alkyl, difluoro(1-6C)alkyl, trifluoro(1-6C)alkyl, Cyc¹, Ar¹, —OAr¹, hetCyc¹, hetAr¹, —OhetAr¹, —CH(OH)CH₂OH, —C(CH₃)₂(OMe) and Br;

Cyc¹ is (3-6C)cycloalkyl optionally substituted with CF₃;

Ar¹ is phenyl optionally substituted with one or more groups independently selected from halogen, CF₃, (1-4C)alkyl and (1-4C)alkoxy;

hetCyc¹ is a 5-6 membered heterocycle having 1-2 ring heteroatoms and optionally substituted with one or more groups independently selected from (1-4C)alkyl;

hetAr¹ is a 5-6-membered heteroaryl having 1-2 ring heteroatoms and optionally substituted with one or more substituents independently selected from (1-4C)alkyl, halogen, CF₃, and (1-4C)alkoxy; and

n is 1, 2 or 3.

In one embodiment of Formula IE, R^{8a} is (1-6C)alkyl, fluoro(1-6C)alkyl, difluoro(1-6C)alkyl or trifluoro(1-6C)alkyl; and R¹, R², R³, R⁴, R⁵ and n are as defined for Formula IA.

In one embodiment of Formula IE, R^{8a} is (1-6C)alkyl, fluoro(1-6C)alkyl, difluoro(1-6C)alkyl or trifluoro(1-6C)alkyl; R¹, R², R³ and R⁴ are independently H, halogen and (1-6C)alkyl; and R⁵ and n are as defined for Formula IE.

It will be appreciated that certain compounds according to the invention may contain one or more centers of asymmetry and may therefore be prepared and isolated as a mixture of isomers such as a racemic or diastereomeric mixture, or in an enantiomerically or diastereomerically pure form. It is intended that all stereoisomeric forms of the compounds of the invention, including but not limited to, diastereomers, enantiomers and atropisomers, as well as mixtures thereof such as racemic mixtures, form part of the present invention.

It may be advantageous to separate reaction products from one another and/or from starting materials. The desired products of each step or series of steps is separated and/or purified (hereinafter separated) to the desired degree of homogeneity by techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Enantiomers can be separated by converting the enantiomeric mixture into a diastereomeric mixture by reaction with an appropriate optically active compound (e.g., chiral auxiliary, such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column. Diastereomeric mixtures can be separated into their individual diastereomers on the basis of their physical chemical differences by methods well known to those skilled in the art, such as by chromatography and/or fractional crystallization.

A single stereoisomer, for example, an enantiomer, substantially free of its stereoisomer may be obtained by resolution of the racemic mixture using methods known in the art, such as (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., ed., Drug Stereochemistry: Analytical Methods and Pharmacology. New York: Marcel Dekker, Inc., 1993.

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid, can result in formation of the diastereomeric salts.

Alternatively, by method (2), the substrate to be resolved is reacted with one enantiomer of a chiral compound to form a diastereomeric pair (Eliel, E., and S. Wilen. Stereochemistry of Organic Compounds. New York: John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III, Peyton. “Resolution of (±)-5-Bromonornicotine. Synthesis of (R)- and (S)-Nornicotine of High Enantiomeric Purity.” J. Org. Chem. Vol. 47, No. 21 (1982): pp. 4165-4167), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (Lough, W. J., ed. Chiral Liquid Chromatography. New York: Chapman and Hall, 1989; Okamoto, Yoshio, et al. “Optical resolution of dihydropyridine enantiomers by high-performance liquid chromatography using phenylcarbamates of polysaccharides as a chiral stationary phase.” J. of Chromatogr. Vol. 513 (1990): pp. 375-378). An example of a chiral stationary phase is a CHIRALPAK ADH column. Enriched or purified enantiomers can be distinguished by methods used to distinguish other chiral molecules with asymmetric carbon atoms, such as optical rotation and circular dichroism.

It will further be appreciated that an enantiomer of a compound of the invention can be prepared by starting with the appropriate chiral starting material.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

Compounds of Formula I include both enantiomers of the position marked with an asterisk (*) as shown below:

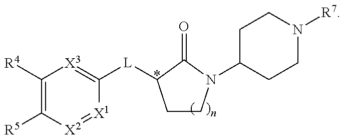

In one embodiment, compound of Formula I have the absolute configuration as shown in Formula I-a

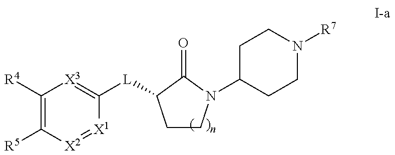

In one embodiment, compound of Formula I have the absolute configuration as shown in Formula I-b:

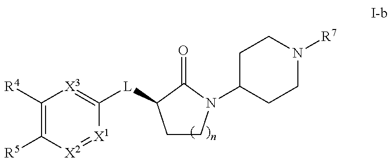

In one embodiment, a compound of Formula I can be enriched in one enantiomer over the other by up to 80% enantiomeric excess. In one embodiment, a compound of Formula I can be enriched in one enantiomer over the other by up to 85% enantiomeric excess. In one embodiment, a compound of Formula I can be enriched in one enantiomer over the other by up to 90% enantiomeric excess. In one embodiment, a compound of Formula I can be enriched in one enantiomer over the other by up to 95% enantiomeric excess.

As used herein, the term “enantiomeric excess” means the absolute difference between the mole fraction of each enantiomer.

The terms “(1-3C)alkyl”, “(1-4C)alkyl” and “(1-6C)alkyl” as used herein refer to saturated linear or branched-chain monovalent hydrocarbon radicals of one to three, one to four, or one to six carbons, respectively.

The term “fluoro(1-6C)alkyl” as used herein refers to saturated linear or branched-chain monovalent radicals of one to six carbon atoms, wherein one of the hydrogen atoms is replaced by fluorine.

The term “difluoro(1-6C)alkyl” as used herein refers to saturated linear or branched-chain monovalent radicals of one to six carbon atoms, wherein two of the hydrogen atoms are replaced by fluorine.

The term “trifluoro(1-6C)alkyl” as used herein refers to saturated linear or branched-chain monovalent radicals of one to six carbon atoms wherein three of the hydrogen atoms are replaced by fluorine.

The terms “(1-4C)alkoxy” and “(1-6C)alkoxy” as used herein refer to saturated linear or branched-chain monovalent alkoxy radicals of one to four or one to six carbon atoms, respectively, wherein the radical is on the oxygen atom.

The term “(1-3C alkyl)sulfonyl” as used herein refers to a (1-3C alkyl)SO₂— group, wherein the radical is on the sulfur atom and the (1-3C alkyl) portion is as defined above.

The term “(3-6C cycloalkyl)sulfonyl” as used herein refers to a (3-6C cycloalkyl)SO₂— group, wherein the radical is on the sulfur atom. The term “(2-6C)dihydroxyalkyl” as used herein refers to saturated linear or branched-chain monovalent hydrocarbon radicals of two to six carbon atoms, respectively, wherein two of the hydrogen atoms are replaced with a OH group, provided that two OH groups are not on the same carbon.

The term “halogen” includes fluoro, chloro, bromo and iodo.

It will also be appreciated that certain compounds of Formula I may be used as intermediates for the preparation of further compounds of Formula I.

The compounds of Formula I include salts thereof. In certain embodiments, the salts are pharmaceutically acceptable salts. In addition, the compounds of Formula I include other salts of such compounds which are not necessarily pharmaceutically acceptable salts, and which may be useful as intermediates for preparing and/or purifying compounds of Formula I and/or for separating enantiomers of compounds of Formula I. Examples of particular salts include trifluoroacetate and hydrochloride salts.

The term “pharmaceutically acceptable” indicates that the substance or composition is compatible chemically and/or toxicologically, with the other ingredients comprising a formulation, and/or the mammal being treated therewith.

It will further be appreciated that the compounds of Formula I and their salts may be isolated in the form of solvates, and accordingly that any such solvate is included within the scope of the present invention.

Compounds of the invention may also contain unnatural proportions of atomic isotopes at one or more of the atoms that constitute such compounds. That is, an atom, in particular when mentioned in relation to a compound according to Formula I, comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof when nitrogen is mentioned, it is understood to refer to ¹³N, ¹⁴N, ¹⁵N or mixtures thereof when oxygen is mentioned, it is understood to refer to ¹⁴O, ¹⁵O, ¹⁶O, ¹⁷O, ¹⁸O or mixtures thereof and when fluoro is mentioned, it is understood to refer to ¹⁸F, ¹⁹F or mixtures thereof. The compounds according to the invention therefore also comprise compounds with one or more isotopes of one or more atom, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds of the present invention, whether radioactive or not, are intended to be encompassed within the scope of the present invention.

The present invention further provides a process for the preparation of a compound of Formula I or a salt thereof as defined herein which comprises:

(a) for a compound of Formula I where L is NR^{x}, coupling a corresponding compound of Formula II

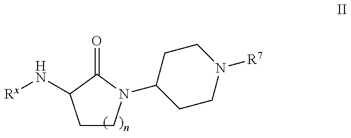

where R^{x}, R⁷ and n are as defined for Formula I, with a corresponding compound having the formula:

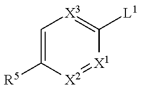

where X¹, X², X³ and R⁵ are as defined for Formula I and L¹ is a leaving group or atom, in the presence of (i) an alkali metal hydride or carbonate or (ii) a palladium catalyst and a ligand; or

(b) for a compound of Formula I where L is O, coupling a corresponding compound of Formula III

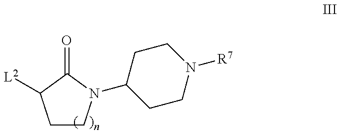

where n and R⁷ are as defined for Formula I and L² is a leaving atom, with a compound having the formula:

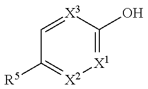

where X¹, X², X³ and R⁵ are as defined for Formula I, in the presence of a base; or

(c) for a compound of Formula I where L is CH₂, coupling a corresponding compound of Formula IV

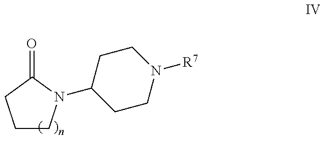

where n and R⁷ are as defined for Formula I, with a compound having the formula:

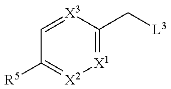

where X¹, X², X³ and R⁵ are as defined for Formula I and L³ is a leaving group of atom, in the presence of a base; or

(d) for a compound of Formula I where R⁷ is

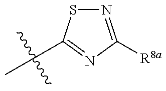

and R^{8a} is as defined for Formula I, reacting a corresponding compound of Formula V

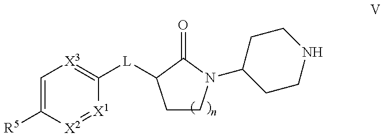

where R⁵, X¹, X², X³, L and n are as defined for Formula I, with a corresponding compound having the formula

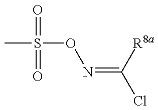

or a protected form thereof, where R^{8a} is as defined for Formula I, in the presence of sodium isothiocyanate and a base; or

(e) for a compound of Formula I where R⁷ is

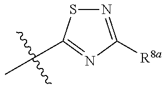

and R^{8a} is as defined for Formula I, reacting a corresponding compound of Formula V

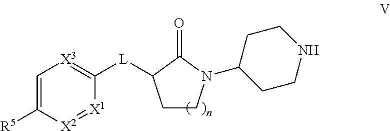

where R⁵, X¹, X², X³, L and n are as defined for Formula I, with a corresponding compound having the formula

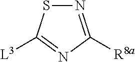

where L³ is a leaving group or atom and R^{8a} is as defined for Formula I, in the presence of a base; or

(f) for a compound of Formula I where R⁵ is (1-3C alkyl)sulfonyl, (3-6C cycloalkyl)sulfonyl, (cyclopropylmethyl)sulfonyl or phenylsulfonyl, reacting a corresponding compound having the Formula VI

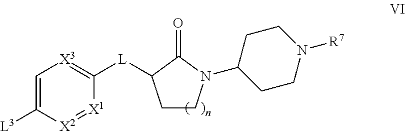

where X¹, X², X³, L, n and R⁷ are as defined for Formula I and L³ is a leaving group or atom, with a compound having the formula R^{y}SO₂Na where R^{y} is (1-3C)alkyl, (3-6C)cycloalkyl, cyclopropylmethyl or phenyl, in the presence of a base and a metal catalyst; or

(g) for a compound of Formula I where R⁵ is (1-3C alkyl)sulfonyl, (3-6C cycloalkyl)sulfonyl, (cyclopropylmethyl)sulfonyl or phenylsulfonyl, treating a corresponding compound having the Formula VII

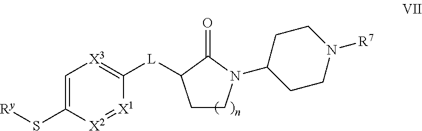

where R^{y} is (1-3C)alkyl, (3-6C)cycloalkyl, cyclopropylmethyl or phenyl, with an oxidizing agent; or

(h) for a compound of Formula I where R⁵ is CN, reacting a corresponding compound of Formula VIII

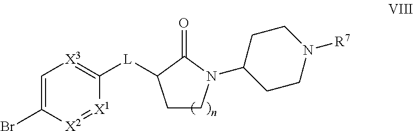

where X¹, X², X³, L, n and R⁷ are as defined for Formula I, with Cu(I)CN; or

(i) for a compound of Formula I where R⁵ is cyclopropylsulfonyl, treating a corresponding compound of Formula IX

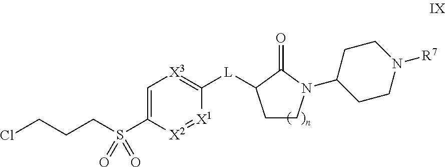

where X¹, X², X³, L, n and R⁷ are as defined for Formula I, with a base; or

(j) for a compound of Formula I where R⁵ is HOCH₂CH₂NHC(═O)— or R′R″NCH₂CH₂NR′″C(═O)— where R′, R″ and R′″ are as defined for Formula I, reacting a compound having the Formula X

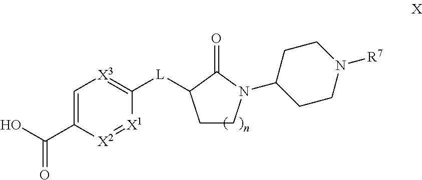

with a corresponding compound having the formula HOCH₂CH₂NH₂ or R′R″NCH₂CH₂NHR′″, respectively, in the presence of a coupling agent; or

(k) for a compound of Formula I where R⁷ is

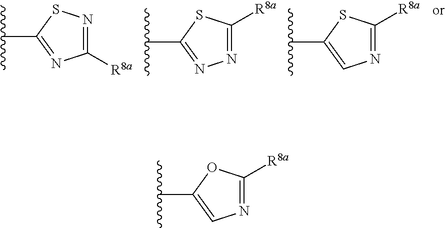

R^{8a} is hetAr¹, R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, and X¹, X², X³, L and n and are as defined for Formula I, coupling a corresponding compound of Formula XI

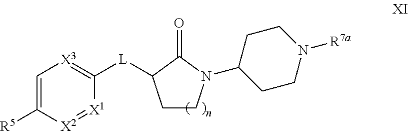

where R^{7a} is

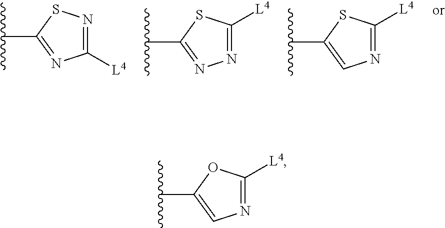

respectively, R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, and X¹, X², X³, L and n and are as defined for Formula I and L⁴ is a leaving atom, with a corresponding compound having the formula

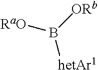

where hetAr¹ is as defined for Formula I and R^{a} and R^{b} are H or (1-6C)alkyl, or R^{a} and R^{b} together with the atoms to which they are connected form a 5-6 membered ring optionally substituted with 1-4 substituents selected from (1-3C alkyl), wherein said coupling takes place in the presence of a palladium catalyst and base and optionally in the presence of a ligand; or

(l) for a compound of Formula I where R⁷ is

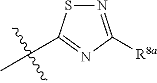

R^{8a} is —OAr¹ or —OhetAr¹, and R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, coupling a corresponding compound of Formula XI

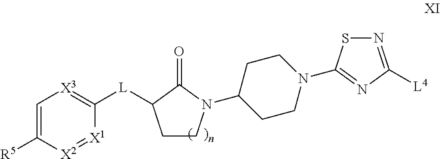

where R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, X¹, X², X³, L and n are as defined for Formula I and L⁴ is a leaving group or atom, with a corresponding compound having the formula HO—Ar¹ or HO-hetAr¹, respectively, in the presence of a base; or

(m) for a compound of Formula I where R⁷ is

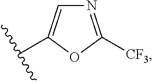 [SMILES: *c1cnc(C(F)(F)F)o1]

reacting a compound of Formula XII

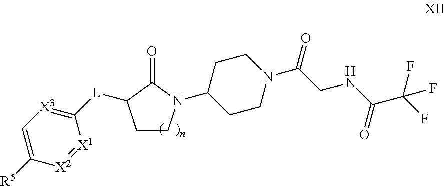

where X¹, X², X³, L, n and R⁵ are as defined for Formula I, with triphenylphosphine and bromine in the presence of a base; or

(n) for a compound of Formula I where R⁷ is

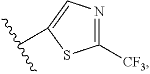 [SMILES: *c1cnc(C(F)(F)F)s1]

reacting a compound of Formula XII

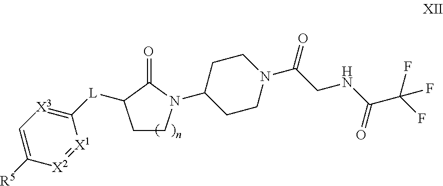

where X¹, X², X³, L, n and R⁵ are as defined for Formula I, with a thiation agent; or

(o) for a compound of Formula I where R⁷ is

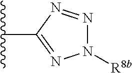

and R^{8b} is (1-6C)alkyl, reacting a corresponding compound having the formula XIII

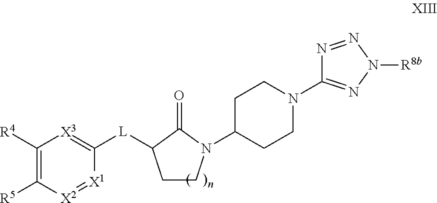

with (1-6C alkyl)OTf in the presence of an acid; and

optionally removing any protecting groups and optionally preparing a pharmaceutically acceptable salt thereof.

Referring to method (a), the leaving atom L¹ may be, for example, a halide such as Br or I. Alternatively, L¹ may be a leaving group, such as a hydrocarbylsulfonyloxy group, for example, a triflate group, or an arylsulfonyloxy group or an alkylsulfonyloxy group, such as a tosylate or a mesylate group. Suitable palladium catalysts include Pd₂(dba)₃ and Pd(OAc)₂. Suitable ligands include Xantphos, rac-BINAP or DIPHOS. The base may be, for example, an alkali metal carbonate or alkoxide, such as for example cesium carbonate or sodium tert-butoxide. Convenient solvents include aprotic solvents such as ethers (for example tetrahydrofuran or p-dioxane) or toluene.

Referring to method (b), the leaving atom L² may be, for example, a halide such as Br or I. Alternatively, L² may be a leaving group, such as a hydrocarbylsulfonyloxy group, for example, a triflate group, or an arylsulfonyloxy group or an alkylsulfonyloxy group, such as a tosylate or a mesylate group. The base may be, for example, an alkali metal hydride or carbonate, such as sodium hydride, potassium hydride, sodium carbonate, potassium carbonate or cesium carbonate. Convenient solvents include aprotic solvents such as ethers (for example tetrahydrofuran or p-dioxane), DMF, or acetone. The reaction can be conveniently performed at a temperature ranging from −78 to 100° C.

Referring to method (c), suitable bases include alkali metal amine bases such as lithium diisopropylamide and silicon-containing alkali metal amides (e.g., sodium hexamethyldisilazide or lithium hexamethyldisilazide). Convenient solvents include aprotic solvents such as ethers (for example tetrahydrofuran or p-dioxane), toluene, DMF or DME. The reaction can be conveniently performed at reduced temperatures, for example at −78° C.

Referring to method (d), suitable bases include amine bases such as pyridine or triethylamine. Suitable solvents include neutral solvents such as acetonitrile, THF, and dichloroethane.

Referring to method (e), the leaving atom L³ may be, for example, a halide such as Br or I. Alternatively, L² may be a leaving group, such as a hydrocarbylsulfonyloxy group, for example, a triflate group, or an arylsulfonyloxy group or an alkylsulfonyloxy group, such as a tosylate or a mesylate group. Suitable bases include amine bases such as triethylamine and diisopropylethylamine, or an alkali metal carbonate, such as potassium carbonate or cesium carbonate. Suitable solvents include alcoholic solvents such as ethanol.

Referring to method (f), the metal catalyst may be a copper or palladium catalyst. An example is copper(I) triflate benzene complex. Suitable bases include amine bases such as trans-cyclohexane-1,2-diamine, triethylamine and diisopropylethylamine.

Referring to method (g), suitable oxidizing agents include 3-chlorobenzoperoxoic acid and m-chloroperbenzoic acid. Suitable solvents include neutral solvents such as acetonitrile, THF, and dichloroethane.

Referring to method (h), the reaction is conveniently performed in an aprotic solvent such as N-methylpyrrolidone (NMP), DMF, DMA or DMSO. The reaction may be performed at elevated temperatures, e.g., >150° C.

Referring to method (i), suitable bases include alkali metal hydrides such as NaH, alkali metal amine bases such as lithium diisopropylamide and silicon-containing alkali metal amides (e.g., sodium hexamethyldisilazide or lithium hexamethyldisilazide).

Referring to method (j), suitable coupling reagents include HATU, HBTU, TBTU, DCC(N,N′-dicyclohexylcarbodiimide), DIEC (1-(3-dimethylaminopropyl)-3-ethylcarboiimide) and any other amide coupling reagents well known to persons skilled in the art.

Referring to method (k), suitable palladium catalysts include P(Cy)₃, PdCl₂(dppf)*dcm, Pd(PPh₃)₄, Pd₂(dba)₃, Pd(OAc)₂, and Pd(PPh₃)₂Cl₂. Suitable ligands include XPHOS, DIPHOS or rac-BINAP. The base may be, for example, cesium fluoride, an alkali metal carbonate, hydroxide, alkoxide or acetate, such as for example cesium carbonate, sodium carbonate, potassium carbonate, sodium hydroxide, sodium tert-butoxide or potassium acetate. Convenient solvents include aprotic solvents such as ethers (for example tetrahydrofuran or p-dioxane), toluene, DMF or DME. The reaction can be conveniently performed at a temperature ranging from ambient temperature to 120° C., for example from 80 to 110° C.

Referring to method (l), the leaving atom L⁴ may be, for example, a halide such as Br or I. Alternatively, L⁴ may be a leaving group, such as a hydrocarbylsulfonyloxy group, for example, a triflate group, or an arylsulfonyloxy group or an alkylsulfonyloxy group, such as a tosylate or a mesylate group. The base may be, for example, an alkali metal hydride or carbonate, such as sodium hydride, potassium hydride, sodium carbonate, potassium carbonate or cesium carbonate. The reaction is conveniently performed in an aprotic solvent such as DMSO.

Referring to method (m), suitable bases include amine bases such as triethylamine and diisopropylethylamine. Suitable solvents include neutral solvents such as dichloroethane.

Referring to method (n), an example of a thiation agent is Lawesson's reagent (2,4-bis(4-methoxyphenyl)-1,3,2,4-dithiadiphosphetane-2,4-disulfide). Convenient solvents include aprotic solvents such as ethers (for example tetrahydrofuran or p-dioxane), toluene, DMF or DME. The reaction can be conveniently performed at elevated temperatures, for example 120° C.

Referring to method (o), suitable acids include mineral acids such as sulfuric acid.

In one embodiment, compounds of formulas II or V where L is NR^{x} and n is 1 can be prepared as shown in general Scheme 1.

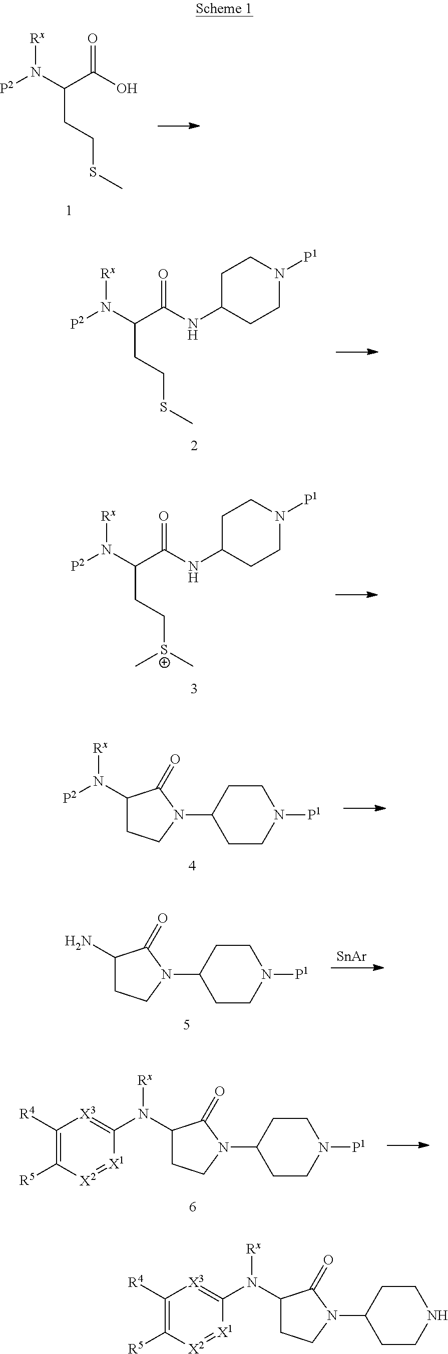

In Scheme 1, P¹ and P² are amine protecting groups. According to Scheme 1, the protected amino piperidine group is coupled to the amino acid intermediate (1) via traditional amide bond forming reagents such as, but not limited to, DCC, to provide compound (2). Compound (2) is activated through methylation reagents such as, but not limited to, methyl iodide to provide compound (3). Cyclization of compound (3) takes place under basic conditions such as, but not limited to, NaH or LHMDS to afford compound (4). Removal of the nitrogen protecting group P² of compound (4) under standard deprotection conditions to provide compound (5), followed by a SnAr reaction with an appropriately functionalized aryl or heteroaryl group provides compounds of formulas II or V where L is NR^{x} after removal of the protecting group P¹ of compound (6) under standard deprotection conditions.

In one embodiment, compounds of formula V where L is O and n is 1, 2 or 3 can be prepared as shown in Scheme 2.

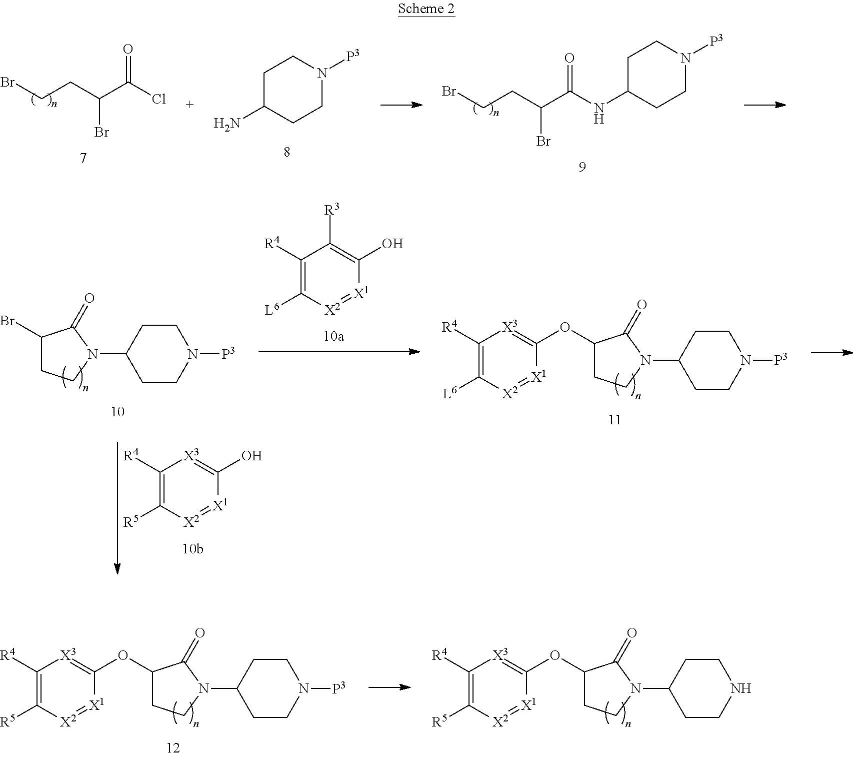

In Scheme 2, P³ is an amine protecting group. According to Scheme 2, acylation of the amino piperidine (8) with acid chloride (7) affords the compound (9). Cyclization of compound (9) to form the lactam (10) is promoted by bases such as, but not limited to, alkali metal hydrides such as NaH, alkali metal amine bases such as lithium diisopropylamide, or silicon-containing alkali metal amides (e.g., sodium hexamethyldisilazide or lithium hexamethyldisilazide). Compound (10) can be coupled with compound (10a) (where L⁶ is a leaving group or atom) under basic conditions, for example, in the presence of an alkali metal hydride or carbonate, such as sodium hydride, potassium hydride, sodium carbonate, potassium carbonate or cesium carbonate. When R⁵ is a group having the R⁵SO₂— where R⁵ is (1-3C) alkyl, (3-6C)cycloalkyl, cyclopropylmethyl- or phenyl, compound (11) can be coupled with a corresponding compound having the formula R⁵SO₂Na in the presence of a metal catalyst such as, but not limited to, copper and palladium catalysts, to provide compound (12). Alternatively, when R⁵ is CN, compound (11) can be reacted with CuCN to provide compound (12). Alternatively, compound (10) can be coupled with compound (10b) to provide compound (12). Removal of the protecting group P³ of compound (12) under standard deprotection conditions affords compounds of formula V where X is O and n is 1, 2 or 3.

In one embodiment, compounds of formula V where L is NR^{x} and n is 2 or 3 can be prepared as shown in Scheme 3.

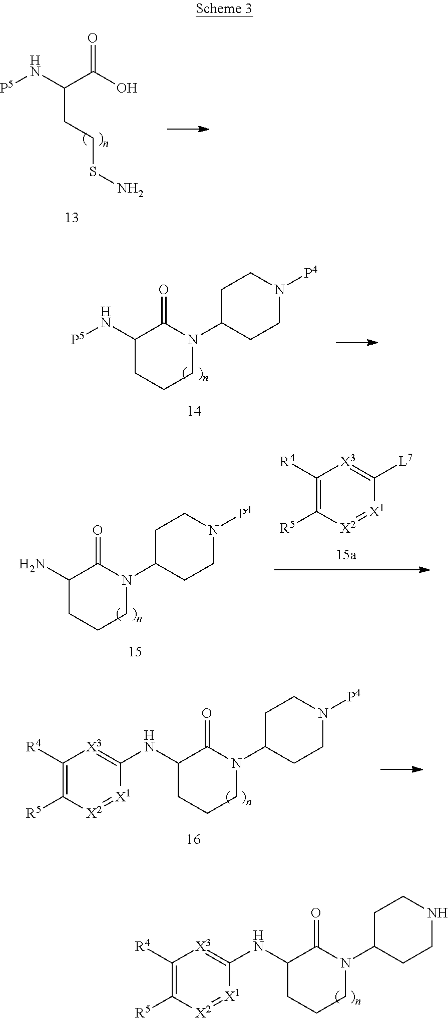

In Scheme 3, P⁴ and P⁵ are amine protecting groups. According to Scheme 3, amino acid (13) is converted to lactam (14) through sequential reductive amination and amide bond formation. Removal of protecting group P⁵ of compound (14) under standard deprotection conditions, followed by coupling of the deprotected compound (15) with compound (15a) under standard SnAr conditions affords intermediate (16). The NH₂ group of compound (15) can optionally be alkylated under standard alkylation conditions known to persons skilled in the art prior to removal of the protecting group P⁴. Removal of the protecting group P⁴ of compound (16) affords compounds of formula V where L is NR^{x} and n is 2 or 3.

Amine groups in compounds described in any of the above methods may be protected with any convenient amine protecting group, for example as described in Greene & Wuts, eds., “Protecting Groups in Organic Synthesis”, 2^{nd} ed. New York; John Wiley & Sons, Inc., 1991. Examples of amine protecting groups include acyl and alkoxycarbonyl groups, such as t-butoxycarbonyl (BOC), and [2-(trimethylsilyl)ethoxy]methyl (SEM). Likewise, carboxyl groups may be protected with any convenient carboxyl protecting group, for example as described in Greene & Wuts, eds., “Protecting Groups in Organic Synthesis”, 2^{nd} ed. New York; John Wiley & Sons, Inc., 1991. Examples of carboxyl protecting groups include (1-6C)alkyl groups, such as methyl, ethyl and t-butyl. Alcohol groups may be protected with any convenient alcohol protecting group, for example as described in Greene & Wuts, eds., “Protecting Groups in Organic Synthesis”, 2^{nd} ed. New York; John Wiley & Sons, Inc., 1991. Examples of alcohol (hydroxyl) protecting groups include benzyl, trityl, silyl ethers, and the like.

The compounds of the formulas II, III, IV, V, VI, VII, VIII, IX, X, XI, XII and XIII are also believed to be novel and are provided as further aspects of the invention.

Compounds of Formula I are modulators of GPR119 and are useful for treating or preventing disease including, but not limited to, type 2 diabetes, diabetic complications, symptoms of diabetes, metabolic syndrome, obesity, dyslipidemia, and related conditions.

The ability of compounds of the invention to act as modulators of GPR119 may be demonstrated by the assay described in Example A.

The term “modulate” refers to the treating, prevention, suppression, enhancement or induction of a function or condition. For example, compounds can modulate Type 2 diabetes by increasing insulin in a human, thereby suppressing hyperglycemia.

The term “modulator” as used herein includes the terms agonist, antagonist, inverse agonist, and partial agonist.

The term “agonist” refers to a compound that binds to a receptor and triggers a response in a cell. An agonist mimics the effect of an endogenous ligand, a hormone for example, and produces a physiological response similar to that produced by the endogenous ligand.

The term “partial agonist” refers to a compound that binds to a receptor and triggers a partial response in a cell. A partial agonist produces only a partial physiological response of the endogenous ligand.

The term “antagonist” as used herein refers to is a type of receptor ligand or drug that does not provoke a biological response itself upon binding to a receptor, but blocks or dampens agonist-mediated responses.

The term “inverse agonist” as used herein refers to an agent that binds to the same receptor binding-site as an agonist for that receptor and reverses constitutive activity of the receptor.

Certain compounds of Formula I are agonists of GPR119.

Certain compounds of Formula I are inverse agonists of GPR119.

Certain compounds of Formula I are antagonists of GPR119.

In certain embodiments, compound of Formula I are useful for treating or preventing type 2 diabetes mellitus (also known as non-insulin dependent diabetes mellitus, or T2DM). Diabetes mellitus is a condition where the fasting plasma glucose level (glucose concentration in venous plasma) is greater than or equal to 126 mg/dL (tested on two occasions) and the 2-hour plasma glucose level of a 75 g oral glucose tolerance test (OGTT) is greater than or equal to 200 mg/dL. Additional classic symptoms include polydipsia, polyphagia and polyuria.

Accordingly, one aspect of the present invention provides methods for treating or preventing type 2 diabetes mellitus in a mammal, comprising administering to the mammal in need of such treatment a therapeutically effective amount of a compound of Formula I or a pharmaceutically acceptable salt thereof.

In certain embodiments, compound of Formula I are useful for treating or preventing diabetic complications. The term “diabetic complications” includes, but is not limited to, microvascular complications and macrovascular complications. Microvascular complications are those complications that generally result in small blood vessel damage. These complications include, for example, retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys). Macrovascular complications are those complications that generally result from large blood vessel damage. These complications include, e.g., cardiovascular disease and peripheral vascular disease. Cardiovascular disease is generally one of several forms, including, e.g., hypertension (also referred to as high blood pressure), coronary heart disease, stroke, and rheumatic heart disease. Peripheral vascular disease refers to diseases of any of the blood vessels outside of the heart. It is often a narrowing of the blood vessels that carry blood to leg and arm muscles.

Accordingly, one aspect of the present invention provides methods for treating or preventing diabetic complications in a mammal, comprising administering to the mammal in need of such treatment a therapeutically effective amount of a compound of Formula I or a pharmaceutically acceptable salt thereof. In one embodiment, the diabetic complication is retinopathy (also known as diabetic retinopathy).

In certain embodiments, compound of Formula I are useful for treating or preventing symptoms of diabetes. The term “symptom” of diabetes, includes, but is not limited to, polyuria, polydipsia, and polyphagia, as used herein, incorporating their common usage. For example, “polyuria” means the passage of a large volume of urine during a given period; “polydipsia” means chronic, excessive thirst; and “polyphagia” means excessive eating. Other symptoms of diabetes include, e.g., increased susceptibility to certain infections (especially fungal and staphylococcal infections), nausea, and ketoacidosis (enhanced production of ketone bodies in the blood).

Accordingly, one aspect of the present invention provides methods for treating or preventing symptoms of diabetes in a mammal, comprising administering to the mammal in need of such treatment a therapeutically effective amount of a compound of Formula I or a pharmaceutically acceptable salt thereof.

In certain embodiments, compound of Formula I are useful for treating or preventing metabolic syndrome in a mammal. The term “metabolic syndrome” refers to a cluster of metabolic abnormalities including abdominal obesity, insulin resistance, glucose intolerance, hypertension and dyslipidemia. These abnormalities are known to be associated with an increased risk of type 2 diabetes and cardiovascular disease. Compounds of Formula I are also useful for reducing the risks of adverse sequelae associated with metabolic syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

Accordingly, one aspect of the present invention provides methods of treating a metabolic syndrome in a mammal, comprising administering to the mammal in need of such treatment a therapeutically effective amount of a compound of Formula I or a pharmaceutically acceptable salt thereof. In one embodiment, the metabolic syndrome is hyperglycemia. In one embodiment, the metabolic syndrome is impaired glucose tolerance. In one embodiment, the metabolic syndrome is insulin resistance. In one embodiment, the metabolic syndrome is atherosclerosis.

In certain embodiments, compound of Formula I are useful for treating or preventing obesity in a mammal. The term “obesity” refers to, according to the World Health Organization, a Body Mass Index (“BMI”) greater than 27.8 kg/m² for men and 27.3 kg/m² for women (BMI equals weight (kg)/height (m²)). Obesity is linked to a variety of medical conditions including diabetes and hyperlipidemia. Obesity is also a known risk factor for the development of Type 2 diabetes.

Accordingly, one aspect of the present invention provides methods of treating or preventing obesity in a mammal, comprising administering to the mammal in need of such treatment a therapeutically effective amount of a compound of Formula I or a pharmaceutically acceptable salt thereof.

Compounds of Formula I may also be useful for treating or preventing diseases and disorders such as, but not limited to, dyslipidemia and dyslipoproteinemia.

The term “dyslipidemia” refers to abnormal levels of lipoproteins in blood plasma including both depressed and/or elevated levels of lipoproteins (e.g., elevated levels of LDL and/or VLDL, and depressed levels of HDL).

The term “dyslipoproteinemia” refers to abnormal lipoproteins in the blood, including hyperlipidemia, hyperlipoproteinemia (excess of lipoproteins in the blood) including type I, II-a (hypercholesterolemia), II-b, III, IV (hypertriglyceridemia) and V (hypertriglyceridemia).

Accordingly, one aspect of the present invention provides methods of treating or preventing dyslipidemia in a mammal, comprising administering to the mammal in need of such treatment a therapeutically effective amount of a compound of Formula I or a pharmaceutically acceptable salt thereof.

Another aspect of the present invention provides methods of treating or preventing dyslipoproteinemia in a mammal, comprising administering to the mammal in need of such treatment a therapeutically effective amount of a compound of Formula I or a pharmaceutically acceptable salt thereof.

By elevating levels of active GLP-1 in vivo, the compounds are useful in treating neurological disorders such as Alzheimer's disease, multiple sclerosis, and schizophrenia.

Accordingly, one aspect of the invention provides methods of treating neurological disorders in a mammal, comprising administering to the mammal in need thereof a therapeutically effective amount of a compound of Formula I or a pharmaceutically acceptable salt thereof. In one embodiment, the neurological disorder is Alzheimer's disease.

Compounds of Formula I generally are useful for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

Accordingly, one aspect of the invention provides methods for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis, comprising administering to the mammal in need of such treatment a therapeutically effective amount of a compound of Formula I or a pharmaceutically acceptable salt thereof. In one embodiment, the disease is selected from type 2 diabetes.

According to another aspect, the invention provides methods for treating or preventing diseases and conditions selected from type 2 diabetes, symptoms of diabetes, diabetic complications, metabolic syndrome (including hyperglycemia, impaired glucose tolerance, and insulin resistance), obesity, dyslipidemia and dyslipoproteinemia.

Compounds of Formula I may also be useful for increasing satiety, reducing appetite, and reducing body weight in obese subjects and may therefore be useful in reducing the risk of co-morbidities associated with obesity such as hypertension, atherosclerosis, diabetes, and dyslipidemia.

Accordingly, the present invention provides methods of inducing satiety, reducing appetite, and reducing body weight in a mammal, comprising administering to the mammal in need thereof a therapeutically effective amount of a compound of Formula I or a pharmaceutically acceptable salt thereof.

In one aspect, the present invention provides methods of inducing satiety in a mammal, comprising administering to the mammal in need thereof a therapeutically effective amount of a compound of Formula I or a pharmaceutically acceptable salt thereof.

In one aspect, the present invention provides methods of decreasing food intake in a mammal, comprising administering to the mammal in need thereof a therapeutically effective amount of a compound of Formula I or a pharmaceutically acceptable salt thereof.

In one aspect, the present invention provides methods of controlling or decreasing weight gain of a mammal, comprising administering to the mammal in need thereof a therapeutically effective amount of a compound of Formula I or a pharmaceutically acceptable salt thereof.

Compounds of Formula I may be administered alone as a sole therapy or can be administered in addition with one or more other substances and/or treatments that work by the same or a different mechanism of action. These agents may be administered with one or more compounds of Formula I as part of the same or separate dosage forms, via the same or different routes of administration, and on the same or different administration schedules according to standard pharmaceutical practice known to one skilled in the art.

Accordingly, compounds of Formula I can be used in combination with a therapeutically effective amount of one or more additional drugs such as insulin preparations, agents for improving insulin resistance (for example PPAR gamma agonists), alpha-glucosidase inhibitors, biguanides (e.g., metformin), insulin secretagogues, dipeptidylpeptidase IV (DPP4) inhibitors (e.g., sitagliptin), beta-3 agonists, amylin agonists, phosphotyrosine phosphatase inhibitors, gluconeogenesis inhibitors, sodium-glucose cotransporter inhibitors, known therapeutic agents for diabetic complications, antihyperlipidemic agents, hypotensive agents, antiobesity agents, GLP-I, GIP-I, GLP-I analogs such as exendins, (for example exenatide (Byetta), exenatide-LAR, and liraglutide), and hydroxysterol dehydrogenase-1 (HSD-I) inhibitors. In one embodiment, a compound of Formula I is used in combination with a biguanide. In one embodiment, a compound of Formula I is used in combination with metformin. In one embodiment, a compound of Formula I is used in combination with metformin for the treatment of type 2 diabetes. In one embodiment, a compound as described in any one of the Examples is used in combination with metformin for the treatment of type 2 diabetes. In one embodiment, a compound of Formula I is used in combination with a DPP4 inhibitor. In one embodiment, a compound of Formula I is used in combination with sitagliptin. In one embodiment, a compound of Formula I is used in combination with sitagliptin for the treatment of type 2 diabetes. In one embodiment, a compound of any one of compounds of the Examples described below is used in combination with sitagliptin for the treatment of type 2 diabetes.

Accordingly, there is provided a method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I or a pharmaceutically acceptable salt thereof, in combination with in combination with a therapeutically effective amount of one or more additional drugs. In one embodiment, the combination is administered for the treatment of type 2 diabetes. In one embodiment, the additional drug is a biguanide. In one embodiment, the additional drug is metformin. In one embodiment, the additional drug is a DPP4 inhibitor. In one embodiment, the additional drug is sitagliptin.

As used herein, terms “treat” or “treatment” mean an alleviation, in whole or in part, of symptoms associated with a disorder or condition as described herein, or slowing, or halting of further progression or worsening of those symptoms. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder, as well as those prone to have the condition or disorder or those in which the condition or disorder is to be alleviated.

As used herein the terms “prevent” or “preventing” means the prevention of the onset, recurrence or spread, in whole or in part, of the disease or condition as described herein, or a symptom thereof.

The terms “effective amount” and “therapeutically effective amount” refer to an amount of compound that, when administered to a mammal in need of such treatment, is sufficient to (i) treat or prevent a particular disease, condition, or disorder, (ii) attenuate, ameliorate, or eliminate one or more symptoms of the particular disease, condition, or disorder, or (iii) prevent or delay the onset of one or more symptoms of the particular disease, condition, or disorder described herein. The amount of a compound of Formula I that will correspond to such an amount will vary depending upon factors such as the particular compound, disease condition and its severity, the identity (e.g., weight) of the mammal in need of treatment, but can nevertheless be routinely determined by one skilled in the art.

As used herein, the term “mammal” refers to a warm-blooded animal that has or is at risk of developing a disease described herein and includes, but is not limited to, guinea pigs, dogs, cats, rats, mice, hamsters, and primates, including humans.

Compounds of the invention may be administered by any convenient route, e.g. into the gastrointestinal tract (e.g. rectally or orally), the nose, lungs, musculature or vasculature, or transdermally or dermally. Compounds may be administered in any convenient administrative form, for example tablets, powders, capsules, solutions, dispersions, suspensions, syrups, sprays, suppositories, gels, emulsions, patches etc. Such compositions may contain components conventional in pharmaceutical preparations, e.g. diluents, carriers, pH modifiers, sweeteners, bulking agents, excipients and further active agents. If parenteral administration is desired, the compositions will be sterile and in a solution or suspension form suitable for injection or infusion. Such compositions form a further aspect of the invention.

In one embodiment, provided herein is a pharmaceutical combination comprising a therapeutically effective amount of: (a) at least one compound of Formula I; and (b) at least one agent selected from one or more additional drugs such as insulin preparations, agents for improving insulin resistance (for example PPAR gamma agonists), alpha-glucosidase inhibitors, biguanides (e.g., metformin), insulin secretagogues, dipeptidylpeptidase IV (DPP4) inhibitors (e.g., sitagliptin), beta-3 agonists, amylin agonists, phosphotyrosine phosphatase inhibitors, gluconeogenesis inhibitors, sodium-glucose cotransporter inhibitors, known therapeutic agents for diabetic complications, antihyperlipidemic agents, hypotensive agents, antiobesity agents, GLP-I, GIP-I, GLP-I analogs such as exendins, (for example exenatide (Byetta), exenatide-LAR, and liraglutide), and hydroxysterol dehydrogenase-1 (HSD-I) inhibitors, for treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, wherein (a) and (b) are in separate dosage forms or in the same dosage form. In one embodiment, the combination comprises (a) and (b) in an amount effective to treat type 2 diabetes, symptoms of diabetes, diabetic complications, or metabolic syndrome (including hyperglycemia, impaired glucose tolerance, and insulin resistance). In one embodiment, the combination comprises (a) and (b) in an amount effective to treat type 2 diabetes.

The term “pharmaceutical combination” as used herein means a product that results from the mixing or combining of more than one active ingredient and includes both fixed and non-fixed combinations of the active ingredients. The term “fixed combination” means that the active ingredients, e.g. (a) a compound of Formula I and (b) another agent, are both administered to a patient simultaneously in the form of a single entity or same dosage form. The term “non-fixed combination” means that the active ingredients, e.g. (a) a compound of Formula I and (b) another agent, are both administered to a patient as separate entities (separate dosage forms) either simultaneously, concurrently or sequentially with no specific time limits, wherein such administration provides therapeutically effective levels of the two compounds in the body of the patient. For a non-fixed combination, the individual combination partners of the combination may be administered separately at different times during the course of therapy or concurrently in divided or single combination forms.

The present invention further provides a pharmaceutical composition, which comprises a compound of Formula I or a pharmaceutically acceptable salt thereof, as defined hereinabove, and a pharmaceutically acceptable carrier, diluent or excipient.

An example of a suitable oral dosage form is a tablet containing about 25 mg, 50 mg, 100 mg, 250 mg, or 500 mg of the compound of the invention compounded with about 90-30 mg anhydrous lactose, about 5-40 mg sodium croscarmellose, about 5-30 mg polyvinylpyrrolidone (“PVP”) K30, and about 1-10 mg magnesium stearate. The powdered ingredients are first mixed together and then mixed with a solution of the PVP. The resulting composition can be dried, granulated, mixed with the magnesium stearate and compressed to tablet form using conventional equipment. An aerosol formulation can be prepared by dissolving the compound, for example 5-400 mg, of the invention in a suitable buffer solution, e.g. a phosphate buffer, adding a tonicifier, e.g., a salt such sodium chloride, if desired. The solution is typically filtered, e.g., using a 0.2 micron filter, to remove impurities and contaminants.

The present invention further provides a compound of Formula I or a pharmaceutically acceptable salt thereof, for use in therapy. In one embodiment, the present invention provides a compound of Formula I or a pharmaceutically acceptable salt thereof, for use in treating a disease or disorder selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis. In one embodiment, the present invention provides a compound of Formula I or a pharmaceutically acceptable salt thereof, for use in the treatment of type 2 diabetes, symptoms of diabetes, diabetic complications, metabolic syndrome (including hyperglycemia, impaired glucose tolerance, and insulin resistance), obesity, dyslipidemia, or dyslipoproteinemia. In one embodiment, the present invention provides a compound of Formula I or a pharmaceutically acceptable salt thereof, for use in the treatment of type 2 diabetes.

In one embodiment, the invention provides a compound of Formula I or a pharmaceutically acceptable salt thereof, for use in the treatment of a disease or disorder selected from type 2 diabetes, symptoms of diabetes, diabetic complications, metabolic syndrome (including hyperglycemia, impaired glucose tolerance, and insulin resistance), obesity, dyslipidemia, and dyslipoproteinemia.

In one embodiment, the invention provides a compound of Formula I or a pharmaceutically acceptable salt thereof, for use in the treatment of type 2 diabetes mellitus in a mammal.

In one embodiment, the invention provides a compound of Formula I or a pharmaceutically acceptable salt thereof, for use in the treatment of diabetic complications in a mammal.

In one embodiment, the invention provides a compound of Formula I or a pharmaceutically acceptable salt thereof, for use in the treatment of symptoms of diabetes in a mammal.

In one embodiment, the invention provides a compound of Formula I or a pharmaceutically acceptable salt thereof, for use in the treatment of metabolic syndrome in a mammal. In one embodiment, the metabolic syndrome is hyperglycemia. In one embodiment, the metabolic syndrome is impaired glucose tolerance. In one embodiment, the metabolic syndrome is insulin resistance. In one embodiment, the metabolic syndrome is atherosclerosis.

In one embodiment, the invention provides a compound of Formula I or a pharmaceutically acceptable salt thereof, for use in the treatment of obesity in a mammal.

In one embodiment, the invention provides a compound of Formula I or a pharmaceutically acceptable salt thereof, for use in the treatment of dyslipidemia in a mammal.

In one embodiment, the invention provides a compound of Formula I or a pharmaceutically acceptable salt thereof, for use in the treatment of dyslipoproteinemia in a mammal.

In one embodiment, the invention provides a compound of Formula I or a pharmaceutically acceptable salt thereof, for use in the treatment of neurological disorders in a mammal. In one embodiment, the neurological disorder is Alzheimer's disease.

In one embodiment, the invention provides a compound of Formula I or a pharmaceutically acceptable salt thereof, for use in inducing satiety in a mammal.

In one embodiment, the invention provides a compound of Formula I or a pharmaceutically acceptable salt thereof, for use in decreasing food intake in a mammal.

In one embodiment, the invention provides a compound of Formula I or a pharmaceutically acceptable salt thereof, for use in controlling or decreasing weight gain in a mammal.

According to a further aspect, the present invention provides the use of a compound of Formula I or a pharmaceutically acceptable salt thereof, in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis.

According to a further aspect, the present invention provides the use of a compound of Formula I or a pharmaceutically acceptable salt thereof, in the treatment of a disease or condition selected from type 2 diabetes, symptoms of diabetes, diabetic complications, metabolic syndrome (including hyperglycemia, impaired glucose tolerance, and insulin resistance), obesity, dyslipidemia, and dyslipoproteinemia,

According to a further aspect, the present invention provides the use of a compound of Formula I or a pharmaceutically acceptable salt thereof, in the treatment of type 2 diabetes mellitus in a mammal.

According to a further aspect, the present invention provides the use of a compound of Formula I or a pharmaceutically acceptable salt thereof, in the treatment of diabetic complications in a mammal.

According to a further aspect, the present invention provides the use of a compound of Formula I or a pharmaceutically acceptable salt thereof, in the treatment of symptoms of diabetes in a mammal.

According to a further aspect, the present invention provides the use of a compound of Formula I or a pharmaceutically acceptable salt thereof, in the treatment of metabolic syndrome in a mammal.

According to a further aspect, the present invention provides the use of a compound of Formula I or a pharmaceutically acceptable salt thereof, in the treatment of metabolic syndrome in a mammal. In one embodiment, the metabolic syndrome is hyperglycemia. In one embodiment, the metabolic syndrome is impaired glucose tolerance. In one embodiment, the metabolic syndrome is insulin resistance. In one embodiment, the metabolic syndrome is atherosclerosis.

According to a further aspect, the present invention provides the use of a compound of Formula I or a pharmaceutically acceptable salt thereof, in the treatment of obesity in a mammal.

According to a further aspect, the present invention provides the use of a compound of Formula I or a pharmaceutically acceptable salt thereof, in the treatment of dyslipidemia in a mammal.

According to a further aspect, the present invention provides the use of a compound of Formula I or a pharmaceutically acceptable salt thereof, in the treatment of dyslipoproteinemia in a mammal.

According to a further aspect, the present invention provides the use of a compound of Formula I or a pharmaceutically acceptable salt thereof, in the treatment of neurological disorders in a mammal. In one embodiment, the neurological disorder is Alzheimer's disease.

According to a further aspect, the present invention provides the use of a compound of Formula I or a pharmaceutically acceptable salt thereof, in inducing satiety in a mammal.

According to a further aspect, the present invention provides the use of a compound of Formula I or a pharmaceutically acceptable salt thereof, in decreasing food intake in a mammal.

According to a further aspect, the present invention provides the use of a compound of Formula I or a pharmaceutically acceptable salt thereof, in controlling or decreasing weight gain in a mammal.

Another embodiment of the present invention provides the use of a compound of Formula I, or a pharmaceutically acceptable salt thereof, in the manufacture of a medicament for treating or preventing type 2 diabetes mellitus in a mammal.

Another embodiment of the present invention provides the use of a compound of Formula I, or a pharmaceutically acceptable salt thereof, in the manufacture of a medicament for treating or preventing diabetic complications.

Another embodiment of the present invention provides the use of a compound of Formula I, or a pharmaceutically acceptable salt thereof, in the manufacture of a medicament for treating or preventing symptoms of diabetes.

Another embodiment of the present invention provides the use of a compound of Formula I, or a pharmaceutically acceptable salt thereof, in the manufacture of a medicament for treating or preventing metabolic syndrome in a mammal.

Another embodiment of the present invention provides the use of a compound of Formula I, or a pharmaceutically acceptable salt thereof, in the manufacture of a medicament for treating or preventing obesity in a mammal.

Another embodiment of the present invention provides the use of a compound of Formula I, or a pharmaceutically acceptable salt thereof, in the manufacture of a medicament for treating or preventing dyslipidemia or dyslipoproteinemia.

Another embodiment of the present invention provides the use of a compound of Formula I, or a pharmaceutically acceptable salt thereof, in the manufacture of a medicament for treating neurological disorders in a mammal.

Another embodiment of the present invention provides the use of a compound of Formula I, or a pharmaceutically acceptable salt thereof, in the manufacture of a medicament for inducing satiety in a mammal.

Another embodiment of the present invention provides the use of a compound of Formula I, or a pharmaceutically acceptable salt thereof, in the manufacture of a medicament for decreasing food intake in a mammal.

Another embodiment of the present invention provides the use of a compound of Formula I, or a pharmaceutically acceptable salt thereof, in the manufacture of a medicament for controlling or decreasing weight gain of a mammal.

In one embodiment, the compound of Formula I is selected from any one of the compounds of Examples 1-67 or a pharmaceutically acceptable salt thereof. In one embodiment, the pharmaceutically acceptable salt is a trifluoroacetate and hydrochloride salts.

### EXAMPLES

The following examples illustrate the invention. In the examples described below, unless otherwise indicated all temperatures are set forth in degrees Celsius. Reagents were purchased from commercial suppliers such as Aldrich Chemical Company, Lancaster, Alfa, Aesar, TCI, Maybridge, or other suitable suppliers, and were used without further purification unless otherwise indicated. THF, DCM, toluene, DMF and dioxane were purchased from commercial vendors and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried or dried under a stream of dry nitrogen.

Column chromatography was done on a Biotage system (Manufacturer: Dyax Corporation) having a silica gel or C-18 reverse phase column, or on a silica SepPak cartridge (Waters), or using conventional flash column chromatography on silica gel, unless otherwise specified.

Abbreviations used herein have the following meanings:

| | |
|---|---|
| ACN | Acetonitrile |
| APCI | Atmospheric Pressure Chemical Ionization |
| Binap | 2,2′-bis(diphenylphosphino)-1,1′-binaphthyl |
| Boc | tert-butoxycarbonyl |
| CDCl₃ | Deuterated Chloroform |
| DCC | N,N′-dicyclohexylcarbodiimide |
| DCM | Dichloromethane |
| DIEA | Diisopropylethylamine |
| DMF | N,N-Dimethylformamide |
| DMSO | Dimethylsulfoxide |
| EDCI | 1-ethyl-3-(3-dimethylaminopropyl) carbodiimide) |
| HOBT | Hydroxybenzotriazole |
| HPLC | High performance liquid chromatography |
| MCPBA | meta-Chloroperoxybenzoic acid |
| NaBH(OAc)₃ | Sodium triacetoxyborohydride |
| Pd₂dba₃ | Tris(dibenzylideneacetone)dipalladium(0) |
| TEA | Triethylamine |
| TFA | Trifluoroacetic acid |
| THF | Tetrahydrofuran |

### Biological Assay

### Example A

### cAMP Production Assay

The assay utilized HEK-293 cells that stably express a modified version of the GPR119 receptor (94% identity to human receptor), under the control of a CMV promoter containing a tet-on element for tetracycline-inducible expression. GPR119 agonist-induced cyclic AMP (cAMP) production was measured in this cell line using the LANCE cAMP kit (Perkin Elmer, Waltham, Mass.). To generate a working stock of cells for the assay, cells were treated overnight with 1 μg/mL doxycycline at 37° C. in the presence of 5% CO₂ to induce receptor expression. Cells were then harvested by enzymatic dissociation with 0.05% trypsin, resuspended in freezing medium (DMEM growth medium with 10% each of fetal bovine serum and DMSO), aliquoted and frozen at −80° C. On the day of the assay, frozen cells were thawed, washed 1× in PBS and resuspended in Hank's buffered salt solution (HBSS) containing 5 mM HEPES, 0.1% BSA and Alexa Fluor 647-conjugated anti-cAMP antibody (diluted 1:100). The cell suspension was then transferred to a Proxiplate Plus white 384-well assay plate (Perkin-Elmer) at 2000 cells/well. Test compounds at final concentrations ranging from 0.2 nM to 10 μM were added to the assay plate, followed by a one-hour incubation at ambient temperature (volume=10 μL/well). DMSO concentration was held constant at 0.5%. After incubation with test compounds, 10 μL, of a detergent buffer containing a biotinylated cAMP/Europium-conjugated streptavidin complex (Europium-labeled cAMP tracer) were added to each well on the assay plate, followed by a 2-hour incubation at ambient temperature. During this incubation cAMP released from lysed cells competes with the Europium-labeled cAMP tracer for binding to the Alexa Fluor 647-conjugated antibody. Agonist-induced cellular cAMP production resulted in increased competition with the Europium-labeled cAMP tracer, leading to a proportional decrease in the time-resolved fluorescence resonance energy transfer (TR-FRET) signal detected by the Perkin-Elmer Envision plate reader. Cellular cAMP levels were then determined by interpolation of raw signal data using a cAMP standard curve. Compounds were determined to have agonist activity if they stimulated a 1.5-fold or greater increase in cAMP relative to basal levels. Results for the compounds of Examples 1-98 are shown in Table A.

TABLE A

| Example | cAMP production in | Fold over |
|---|---|---|
| No. | HEK-293 cells (nMol) | baseline |
| 1 | 21.6 | 3.3 |
| 2 | 21.9 | 3.1 |
| 3 | 25.3 | 5.4 |
| 4 | 24.2 | 4.3 |
| 5 | 19.2 | 2.7 |
| 6 | 23.2 | 3.2 |
| 7 | 23.8 | 3.4 |
| 8 | 37.6 | 4.7 |
| 9 | 16.0 | 4.0 |
| 10 | 11.3 | 2.3 |
| 11 | 10.8 | 3.0 |
| 12 | 17.5 | 3.9 |
| 13 | 25.2 | 3.4 |
| 14 | 17.5 | 2.8 |
| 15 | 11.4 | 2.7 |
| 16 | 27.2 | 3.7 |
| 17 | 11.2 | 2.1 |
| 18 | 20.1 | 3.4 |
| 19 | 16.4 | 2.4 |
| 20 | 22.6 | 3.5 |
| 21 | 13.0 | 3.3 |
| 22 | 22.1 | 7.6 |
| 23 | 11.9 | 2.9 |
| 24 | 9.7 | 2.6 |
| 25 | 8.3 | 2.1 |
| 26 | 17.2 | 3.9 |
| 27 | 17.5 | 3.0 |
| 28 | 11.6 | 2.3 |
| 29 | 7.3 | 2.2 |
| 30 | 13.9 | 2.8 |
| 31 | 7.6 | 1.9 |
| 32 | 17.8 | 2.7 |
| 33 | 16.9 | 2.7 |
| 34 | 29.2 | 4.5 |
| 35 | 26.1 | 3.7 |
| 36 | 23.2 | 4.1 |
| 37 | 20.9 | 3.2 |
| 38 | 42.1 | 6.6 |
| 39 | 22.4 | 3.7 |
| 40 | 21.1 | 3.9 |
| 41 | 8.1 | 2.0 |
| 42 | 12.0 | 2.5 |
| 43 | 29.4 | 7.4 |
| 44 | 23.8 | 3.4 |
| 45 | 17.8 | 3.5 |
| 46 | 20.7 | 4.7 |
| 47 | 9.8 | 2.7 |
| 48 | 20.5 | 3.3 |
| 49 | 15.4 | 2.8 |
| 50 | 15.1 | 5.6 |
| 51 | 33.3 | 5.9 |
| 52 | 24.6 | 3.9 |
| 53 | 24.5 | 4.9 |
| 54 | 17.2 | 3.6 |
| 55 | 17.2 | 3.8 |
| 56 | 17.4 | 3.4 |
| 57 | 22.0 | 2.8 |
| 58 | 48.4 | 4.9 |
| 59 | 28.3 | 4.2 |
| 60 | 13.2 | 4.5 |
| 61 | 23.5 | 3.5 |
| 62 | 17.4 | 2.7 |
| 63 | 7.4 | 2.7 |
| 64 | 30.8 | 3.5 |
| 65 | 22.1 | 4.1 |
| 66 | 8.6 | 1.8 |
| 67 | 14.0 | 2.3 |
| 68 | 14.5 | 3.1 |
| 69 | 10.6 | 2.1 |
| 70 | 11.4 | 3.1 |
| 71 | 5.9 | 2.1 |
| 72 | 10.6 | 2.9 |
| 73 | 6.2 | 2.2 |
| 74 | 15.3 | 4.2 |
| 75 | 6.9 | 2.7 |
| 76 | 5.7 | 2.5 |
| 77 | 15.1 | 2.8 |
| 78 | 5.0 | 1.6 |
| 79 | 19.0 | 3.2 |
| 80 | 16.1 | 2.8 |
| 81 | 11.7 | 2.4 |
| 82 | 12.8 | 2.4 |
| 83 | 14.9 | 2.5 |
| 84 | 18.8 | 4.2 |
| 85 | 16.2 | 3.8 |
| 86 | 11.1 | 3.4 |
| 87 | 13.6 | 2.1 |
| 88 | 11.8 | 3.0 |
| 89 | 15.1 | 3.8 |
| 90 | 8.5 | 2.9 |
| 91 | 25.6 | 5.4 |
| 92 | 18.4 | 2.8 |
| 93 | 9.0 | 2.9 |
| 94 | 28.1 | 4.0 |
| 95 | 16.7 | 3.4 |
| 96 | 14.2 | 3.1 |
| 97 | 19.0 | 4.1 |
| 98 | 23.7 | 5.4 |

### Preparation A

### N-(methylsulfonyloxy)isobutyrimidoyl chloride

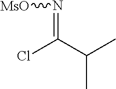

Step A: To a solution of K₂CO₃ (48 g, 347 mmol) in water was added hydroxylamine hydrochloride (48 g, 693 mmol). The reaction was stirred for 5 minutes. Isobutyraldehyde (63 mL, 693 mmol) was added and the reaction was stirred at ambient temperature overnight. The reaction was poured into MTBE and the layers were separated. The combined organic layers were washed with brine, dried over MgSO₄ and concentrated in vacuo to afford crude isobutyraldehyde oxime (54 g, 89% yield).

Step B: To a solution of isobutyraldehyde oxime (54 g, 620 mmol) in DMF at 0° C. was added 1-chloropyrrolidine-2,5-dione (83 g, 620 mmol) and the reaction was stirred overnight, slowly warming to ambient temperature. The reaction was poured into water and extracted into MTBE. The combined organic layers were washed with water, brine, dried over MgSO₄ and concentrated in vacuo to afford crude N-hydroxyisobutyrimidoyl chloride (66.9 g, 89% yield).

Step C: To a solution of N-hydroxyisobutyrimidoyl chloride (66.9 g, 550 mmol) in CH₂Cl₂ cooled to 0° C. was added methanesulfonyl chloride (42.9 mL, 550 mmol) and the reaction mixture was held at 0° C. for 5 minutes. N-ethyl-N-isopropylpropan-2-amine (101 mL, 550 mmol) was added in small portions (exotherm) and then the reaction was cooled to 0° C. The reaction was stirred at 0° C. for 2 hours. The reaction was concentrated in vacuo, ether was added to the residue, and the slurry was stirred for 30 minutes. The suspension was filtered and the solids were washed with ether. The combined organic layers were concentrated in vacuo and the residue was purified over silica gel (5:1 hexanes/EtOAc) to yield an oil. The oil was dissolved in 3% EtOAc/Hexanes to precipitate the product. The solids were filtered and dried to afford N-(methylsulfonyloxy)isobutyrimidoyl chloride (45 g, 41% yield) as a white solid.

The following compounds were also prepared according to the procedure for Preparation A.

| Preparation | Structure | Name |
|---|---|---|
| B | 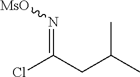 | 3-methyl-N- (methylsulfonyloxy) butanimidoyl chloride |
| C | 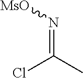 | N-(methylsulfonyloxy) acetimidoyl chloride |
| D | 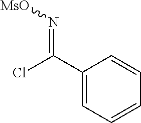 [SMILES: CS(=O)(=O)ON=C(Cl)c1ccccc1] | N-(methylsulfonyloxy) benzimidoyl chloride |
| E | 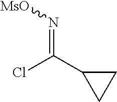 [SMILES: CS(=O)(=O)ON=C(Cl)C1CC1] | N-(methylsulfonyloxy) cyclopropanecarbimidoyl chloride |
| F | 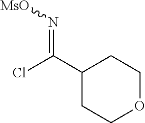 [SMILES: CS(=O)(=O)ON=C(Cl)C1CCOCC1] | N-(methylsulfonyloxy) tetrahydro-2H-pyran-4- carbimidoyl chloride |
| G | 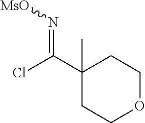 | 4-methyl-N- (methylsulfonyloxy) tetrahydro-2H- pyran-4-carbimidoyl chloride |
| H | 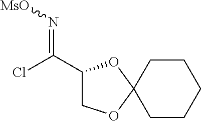 [SMILES: CS(=O)(=O)ON=C(Cl)[C@H]1COC2(CCCCC2)O1] | (R)-N- (methylsulfonyloxy)-1,4- dioxaspiro[4.5]decane- 2-carbimidoyl chloride |

### Preparation I

### 5-chloro-3-(2-methoxypropan-2-yl)-1,2,4-thiadiazole

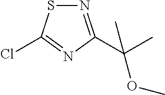

Step A:

Zinc(II) chloride (29.8 g, 219 mmol) was dried under vacuum at 80° C. overnight. The powder was cooled to ambient temperature, purged with nitrogen, and methanol (20 mL, 493 mmol) was added quickly (exothermic). Once the suspension cooled to ambient temperature, 2-hydroxy-2-methylpropanenitrile (20 mL, 219 mmol) was added and the reaction was heated to 60° C. overnight. After cooling to ambient temperature, the reaction was poured onto ice, extracted with Et₂O (3×50 mL), dried over Na₂SO₄, filtered and carefully concentrated (the boiling point of the product is 117° C.). The residue was dissolved in CH₂Cl₂, dried over Na₂SO₄, filtered and carefully concentrated again to afford 2-methoxy-2-methylpropanenitrile (22.4 g, 158 mmol, 72.4% yield) as a clear, colorless oil. The crude material was used in the next reaction without further purification.

Step B:

NH₄Cl (42.4 g, 792 mmol) was suspended in dry toluene (400 mL) under nitrogen and cooled to 0° C. Trimethylaluminum (396 mL, 792 mmol; 2M) was added dropwise and the reaction was slowly allowed to warm to ambient temperature until there was no more gas evolution. 2-Methoxy-2-methylpropanenitrile (15.7 g, 158 mmol) was added and the reaction was heated to 80° C. overnight. The reaction was cooled to 0° C. and methanol (200 mL) was added with constant stirring. The mixture was stirred at ambient temperature for 1 hour. The resulting solids were filtered and washed with methanol several times. The combined filtrates were concentrated in vacuo to afford 2-methoxy-2-methylpropanimidamide hydrochloride (13.8 g, 90.4 mmol, 57.1% yield) as a white solid.

Step C:

2-Methoxy-2-methylpropanimidamide hydrochloride (2.0 g, 13 mmol) and trichloromethyl hypochlorothioite (1.3 mL, 12 mmol) were dissolved in CH₂Cl₂ (15 mL) and cooled to −15° C. Sodium hydroxide (2.6 g, 66 mmol) dissolved in water (5 mL) was added dropwise and the reaction was stirred at ambient temperature for 3 hours. The reaction was diluted with CH₂Cl₂ (15 mL), washed with water, dried over MgSO₄, filtered and concentrated in vacuo to afford crude 5-chloro-3-(2-methoxypropan-2-yl)-1,2,4-thiadiazole (2.1 g, 11 mmol, 91% yield) as an orange oil.

The following compounds were prepared according to Step C of Preparation I.

| Preparation | Structure | Name |
|---|---|---|
| J | 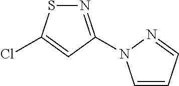 [SMILES: Clc1cc(-n2cccn2)ns1] | 5-chloro-3-(1H-pyrazol- 1-yl)-1,2,4-thiadiazole |
| K | 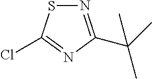 | 3-tert-butyl-5-chloro- 1,2,4-thiadiazole |

### Preparation L

### 1-(3-isopropyl-1,2,4-thiadiazol-5-yl)piperidin-4-amine hydrochloride

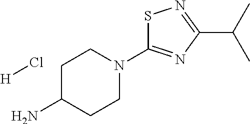

Step A:

To a solution of N-(methylsulfonyloxy)isobutyrimidoyl chloride (20.8 g, 104 mmol) in EtOAc (500 mL) was added NaNCS (8.45 g, 104 mmol) and pyridine (28.0 mL, 348 mmol). The reaction mixture was heated to 45° C. for 45 min. Tert-butyl piperidin-4-ylcarbamate (17.4 g, 86.9 mmol) was added and the reaction was heated to 70° C. for 4 hours and then stirred at ambient temperature overnight. The solids were filtered and the filtrate was concentrated in vacuo. The residue was purified over silica gel (25% EtOAc in hexanes) to afford tert-butyl 1-(3-isopropyl-1,2,4-thiadiazol-5-yl)piperidin-4-ylcarbamate (16.5 g, 50.5 mmol, 58.2% yield) as a pale yellow solid.

Step B:

To a solution of tert-butyl 1-(3-isopropyl-1,2,4-thiadiazol-5-yl)piperidin-4-ylcarbamate (16.5 g, 50.5 mmol) in CH₂Cl₂ (200 mL) and MeOH (50 mL) was added 4N HCl in dioxane (100 mL) and the reaction was stirred at ambient temperature overnight. Et₂O (200 mL) was added and the resulting solids were filtered, washed with ether and hexanes and dried in a vacuum oven to afford 1-(3-isopropyl-1,2,4-thiadiazol-5-yl)piperidin-4-amine hydrochloride (12.5 g, 47.6 mmol, 94.1% yield) as a white solid. Mass spectrum (apci) m/z=227.2 (M+H-HCl).

### Preparation M

### 5-(4-aminopiperidin-1-yl)-N,N-dimethyl-1,2,4-thiadiazol-3-amine

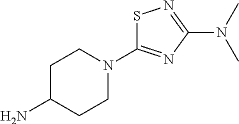

Step A:

To a solution of tert-butyl piperidin-4-ylcarbamate (3.9 g, 19.5 mmol in THF (80 mL) and triethylamine (2.99 mL, 21.4 mmol) and was added a solution of 3,5-dichloro-1,2,4-thiadiazole (3.32 g, 21.4 mmol) in THF (40 mL). An immediate precipitate was formed accompanied by a mild exotherm. The mixture was stirred until it returned to ambient temperature. The mixture was filtered and concentrated in vacuo to afford tert-butyl 1-(3-chloro-1,2,4-thiadiazol-5-yl)piperidin-4-ylcarbamate (5.9 g, 18.5 mmol, 95.0% yield) as a white solid.

Step B:

Tert-butyl 1-(3-chloro-1,2,4-thiadiazol-5-yl)piperidin-4-ylcarbamate (5.9 g, 19 mmol) was dissolved in DMF (90 mL) in a sealed tube and cooled to −10° C. Anhydrous dimethylamine was bubbled through the solution until it had increased in volume about 25%. The reaction was sealed and heated to 100° C. for 4 hours and allowed to cool to ambient temperature overnight. The reaction was further cooled in an ice bath before opening the sealed tube. Nitrogen was bubbled through the mixture for 30 minutes at ambient temperature to remove excess dimethylamine. The reaction was poured into water and the resulting precipitate was filtered, dissolved in EtOAc, dried over Na₂SO₄, and concentrated in vacuo to afford tert-butyl 1-(3-(dimethylamino)-1,2,4-thiadiazol-5-yl)piperidin-4-ylcarbamate (5.7 g, 17 mmol, 94% yield) as a white solid.

Step C:

To a solution of tert-butyl 1-(3-(dimethylamino)-1,2,4-thiadiazol-5-yl)piperidin-4-ylcarbamate (5.7 g, 17 mmol) in CH₂Cl₂ (100 mL) was added TFA (20 mL) and the mixture was stirred at ambient temperature for 30 minutes. The reaction was concentrated in vacuo, partitioned between saturated aqueous NaHCO₃ and EtOAc, extracted with EtOAc (3×100 mL), dried over Na₂SO₄, and concentrated in vacuo to afford 5-(4-aminopiperidin-1-yl)-N,N-dimethyl-1,2,4-thiadiazol-3-amine (3.9 g, 17 mmol, 99% yield) as an oil that slowly solidified into a white solid. Mass spectrum (apci) m/z=228.2 (M+H).

### Preparation N

### 1-bromo-2-fluoro-5-methyl-4-(methylsulfonyl)benzene

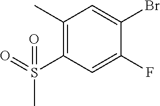

Step A:

4-Bromo-5-fluoro-2-methylaniline (5 g, 24.5 mmol) was dissolved in 1,2-dimethyldisulfane (35 mL, 394 mmol) and heated to 75° C. under nitrogen. Isoamyl nitrite (8.52 mL, 63.7 mmol) was added dropwise to the reaction via an addition funnel through a reflux condenser (about 1 drop/sec). After addition was complete, the reaction was heated to 95° C. for 1 hour and allowed to cool to ambient temperature. The reaction was concentrated and purified over silica gel (100% hexanes) to afford (4-bromo-5-fluoro-2-methylphenyl)(methyl)sulfane (4.9 g, 20.8 mmol, 85.0% yield) as an orange solid.

Step B:

(4-Bromo-5-fluoro-2-methylphenyl)(methyl)sulfane (4.9 g, 21 mmol) was dissolved in CH₂Cl₂ (200 mL) and cooled on an ice bath. 70% MCPBA (11 g, 46 mmol) was added and the reaction was allowed to stir at 0° C. for 15 minutes and then warmed to ambient temperature. The reaction was stirred at ambient temperature for 2 hours, filtered and concentrated in vacuo. The crude mixture was purified over silica gel (30% EtOAc in hexanes) to afford 1-bromo-2-fluoro-5-methyl-4-(methylsulfonyl)benzene (5.4 g, 20 mmol, 97% yield) as a white solid.

### Preparation 0

### 4-bromo-2-fluoro-5-methylphenol

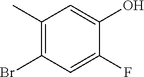

To a solution of 2-fluoro-5-methylphenol (5.0 g, 40 mmol) in chloroform (200 mL) was added tetrabutylammonium tribromide (19 g, 40 mmol) and the reaction was stirred at ambient temperature for 30 minutes. The reaction was concentrated in vacuo and purified over silica gel plug (20% EtOAc in hexanes) to afford 4-bromo-2-fluoro-5-methylphenol (7.8 g, 38 mmol, 96% yield) as an amber oil.

### Preparation P

### 3,4-difluoro-N,N-dimethylbenzenesulfonamide

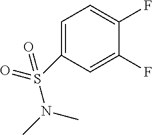

To a solution of 3,4-difluorobenzene-1-sulfonyl chloride (5.0 g, 23.6 mmol) in DMF (100 mL) cooled to 0° C. was added dropwise dimethyl amine (40% solution in water, 2 mL) and the reaction was stirred for 2 hours while warming to ambient temperature. The reaction was poured into diethyl ether and the combined organic layers were washed with water, 1M HCl, brine, dried over magnesium sulfate, filtered and concentrated in vacuo to give 3,4-difluoro-N,N-dimethylbenzenesulfonamide (1.30 g, 5.9 mmol, 25%).

### Preparation Q

### 4-(cyclopropylsulfonyl)-1,2-difluorobenzene

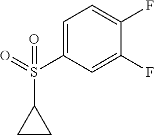 [SMILES: O=S(=O)(c1ccc(F)c(F)c1)C1CC1]

Step A:

To a solution of sodium sulfite (67 g, 529 mmol) in water (200 mL) was added a solution of 3,4-difluorobenzene-1-sulfonyl chloride (15 g, 71 mmol) in dioxanes (100 mL) dropwise. After complete addition of the sulfonyl chloride, the reaction was basified to pH 14 by the addition of 1N sodium hydroxide, and the reaction was stirred overnight at ambient temperature. The reaction was cooled to 0° C. and acidified to pH 1 by addition of concentrated HCl. The reaction was poured into EtOAc and the organic layer was separated. The organics were dried over MgSO₄ and concentrated in vacuo to yield 3,4-difluorobenzenesulfinic acid (13 g, 100%).

Step B:

To a solution of 3,4-difluorobenzenesulfinic acid (2.5 g, 14.0 mmol) in dimethyl formamide (20 mL) was added 1-chloro-3-iodopropane (4.46 mL, 42.1 mmol) and N-ethyl-N-isopropylpropan-2-amine (2.82 mL, 15.4 mmol) and the reaction was stirred overnight at ambient temperature. The reaction was poured into water and extracted into diethyl ether. The combined organic layers were washed with water, brine, dried over MgSO₄, filtered and concentrated in vacuo. The material was purified over silica gel (4:1 hexanes/EtOAc) to yield 4-(3-chloropropylsulfonyl)-1,2-difluorobenzene (2.8 g, 80%).

Step C:

To a solution of 4-(3-chloropropylsulfonyl)-1,2-difluorobenzene (2.8 g, 11.0 mmol) in THF (100 mL) cooled to −78° C. was added potassium hexamethyl disilylazide (12.1 mL, 12.1 mmol, 1M solution in THF) and the reaction was stirred for 1 hour at −78° C. Water was added to the reaction at −78° C. to quench the reaction. The reaction was poured into EtOAc and the combined organic layers were washed with water, brine, dried over MgSO₄, filtered and concentrated in vacuo. The material was purified over silica gel (100% dichloromethane) to yield 4-(cyclopropylsulfonyl)-1,2-difluorobenzene (1.89 g, 78%).

### Preparation R

### 1,2,4-trifluoro-5-(methylsulfonyl)benzene

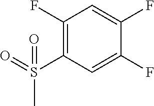

Step A:

To a solution of sodium sulfite (153 g, 1214 mmol) in water (1000 mL) was added a solution of 2,4,5-trifluorobenzene-1-sulfonyl chloride (40 g, 173 mmol) in dioxane (300 mL) dropwise. After the complete addition of sulfonyl chloride, the reaction was basified to pH 14 by the addition of 1N NaOH, and the reaction mixture was stirred overnight. The reaction mixture was cooled on an ice bath and acidified with 100 mL concentrated H₂SO₄ to pH 1. The mixture was extracted with EtOAc and CH₂Cl₂ and the combined organic layers were dried over Na₂SO₄, filtered and concentrated in vacuo to afford 2,4,5-trifluorobenzenesulfinic acid (34 g, 100%).

Step B:

To a solution of 2,4,5-trifluorobenzenesulfinic acid (34 g, 173 mmol) in DMF (200 mL) was added iodomethane (21.6 mL, 347 mmol) and N-ethyl-N-isopropylpropan-2-amine (60.5 mL, 347 mmol). The reaction mixture was stirred overnight at ambient temperature. The reaction was concentrated in vacuo, partitioned between water/ethyl acetate and extracted with CH₂Cl₂. The combined organic layers were concentrated in vacuo and purified over silica gel (15-100% EtOAc in hexanes) to afford 1,2,4-trifluoro-5-(methylsulfonyl)benzene (25.8 g, 123 mmol, 70.8% yield) as yellow solid.

The following compounds were also prepared according to the procedure above.

| Preparation | Structure | Name |
|---|---|---|
| S | 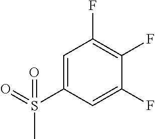 | 1,2,3-trifluoro-5- (methylsulfonyl)benzene |
| T | 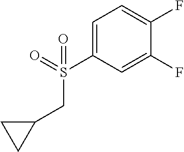 [SMILES: O=S(=O)(CC1CC1)c1ccc(F)c(F)c1] | 4- (cyclopropylmethylsulfonyl)- 1,2-difluorobenzene |
| U | 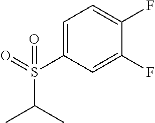 | 1,2-difluoro-4- (isopropylsulfonyl)benzene |
| V | 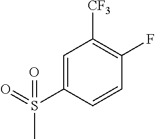 | 1-fluoro-4- (methylsulfonyl)-2- (trifluoromethyl)benzene |
| W | 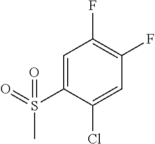 | 1-chloro-4,5-difluoro-2- (methylsulfonyl)benzene |
| X | 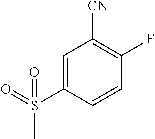 | 2-fluoro-5- (methylsulfonyl) benzonitrile |
| Y | 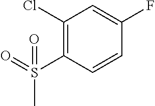 | 2-chloro-4-fluoro-1- (methylsulfonyl)benzene |
| Z | 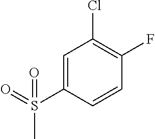 | 2-chloro-1-fluoro-4- (methylsulfonyl)benzene |
| AA | 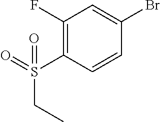 | 4-bromo-1- (ethylsulfonyl)-2- fluorobenzene |
| BB | 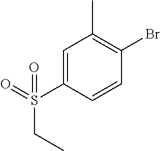 | 1-bromo-4- (ethylsulfonyl)-2- methylbenzene |
| CC | 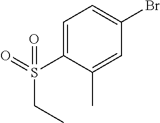 | 4-bromo-1- (ethylsulfonyl)-2- methylbenzene |
| DD | 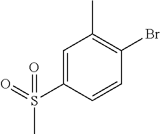 | 1-bromo-2-methyl-4- (methylsulfonyl)benzene |

### Preparation EE

### (R)-tert-butyl 4-(3-(methylsulfonyloxy)-2-oxopyrrolidin-1-yl)piperidine-1-carboxylate

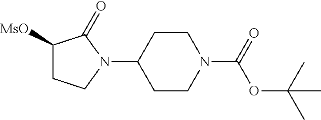 [SMILES: CC(C)(C)OC(=O)N1CCC(N2CC[C@@H](OS(C)(=O)=O)C2=O)CC1]

Step A:

(R)-2-(2,2-dimethyl-5-oxo-1,3-dioxolan-4-yl)acetic acid (25 g, 144 mmol) was dissolved in CH₂Cl₂ (500 mL) and cooled in an ice bath. Ethanethiol (21.2 mL, 287 mmol) and N,N-dimethylpyridin-4-amine (0.351 g, 2.87 mmol) were added followed by DCC (35.5 g, 172 mmol). This mixture was stirred on an ice bath for 1 hour, and then 2 hours at ambient temperature. Acetic acid (45 mL) was added and the mixture was stirred for 10 minutes. The reaction mixture was poured into vigorously stirred ether (400 mL) and filtered. The filtrate was washed with 10% sodium carbonate, water, 0.5 N HCl, water and brine. The organic layer was dried over Na₂SO₄, filtered and concentrated in vacuo. The residue was purified over silica gel (1-5-10% EtOAc in hexanes) to afford (R)—S-ethyl 2-(2,2-dimethyl-5-oxo-1,3-dioxolan-4-yl)ethanethioate (22.5 g, 103 mmol, 71.8% yield) as a clear colorless oil that solidified to a white solid.

Step B:

A suspension of (R)—S-ethyl 2-(2,2-dimethyl-5-oxo-1,3-dioxolan-4-yl)ethanethioate (22.5 g, 103 mmol) and 10% palladium on carbon (2.19 g, 2.06 mmol) in CH₂Cl₂ (500 mL) was purged with nitrogen. A solution of triethylsilane (24.7 mL, 155 mmol in CH₂Cl₂ (20 mL) was added dropwise through an addition funnel over 30 minutes and the mixture was stirred under nitrogen at ambient temperature overnight. The reaction was filtered through Celite®, concentrated in vacuo and purified over silica gel (10 to 40% EtOAc in hexanes) to afford (R)-2-(2,2-dimethyl-5-oxo-1,3-dioxolan-4-yl)acetaldehyde (16 g, 101 mmol, 98.1% yield) as a clear colorless oil.

Step C:

(R)-2-(2,2-dimethyl-5-oxo-1,3-dioxolan-4-yl)acetaldehyde (16 g, 101 mmol) was dissolved in ClCH₂CH₂Cl (500 mL) and tert-butyl 4-aminopiperidine-1-carboxylate (40.5 g, 202 mmol) and acetic acid (6.94 mL, 121 mmol) were added. The mixture was stirred at ambient temperature for 15 minutes. NaBH(OAc)₃ (64.3 g, 304 mmol) was added in 3 portions and the reaction was stirred at ambient temperature overnight. The reaction was carefully quenched with saturated aqueous NaHCO₃. The reaction was partitioned between aqueous NaHCO₃ and CH₂Cl₂, and the combined organic layers were washed with 10% citric acid, brine, dried over Na₂SO₄, filtered and concentrated in vacuo. The resulting solids were purified over silica gel (5 to 10% methanol in EtOAc) to afford (R)-tert-butyl 4-(3-hydroxy-2-oxopyrrolidin-1-yl)piperidine-1-carboxylate (20.5 g, 72.1 mmol, 71.3% yield) as a white solid.

Step D:

To a solution of (R)-tert-butyl 4-(3-hydroxy-2-oxopyrrolidin-1-yl)piperidine-1-carboxylate (20.5 g, 72.1 mmol) in THF (500 mL) was added triethylamine (20.1 mL, 144 mmol) and methanesulfonyl chloride (6.74 mL, 86.5 mmol) After stirring at ambient temperature for 1 hour, the reaction was partitioned between saturated aqueous NaHCO₃ and EtOAc, dried over Na₂SO₄, filtered and concentrated in vacuo. The residue was purified over silica gel to afford (R)-tert-butyl 4-(3-(methylsulfonyloxy)-2-oxopyrrolidin-1-yl)piperidine-1-carboxylate (25.5 g, 70.4 mmol, 97.6% yield) as a white solid. ¹H NMR (CDCl₃) 5.2 ppm (t, 1H), 4.3 ppm (m, 2H), 4.1 ppm (m, 1H), 3.4 ppm (m, 1H), 3.3 ppm (m, 1H), 3.3 ppm (s, 3H), 2.8 ppm (m, 2H), 2.6 ppm (m, 1H), 2.3 ppm (m, 1H), 1.7 ppm (m, 2H, 1.6 ppm (m, 2H), 1.5 ppm (s, 9H).

### Preparation FF

### (S)-3-(2-fluoro-4-(methylsulfonyl)phenylamino)-1-(piperidin-4-yl)pyrrolidin-2-one

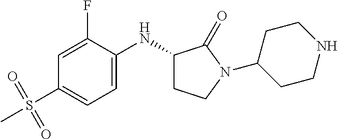 [SMILES: CS(=O)(=O)c1ccc(N[C@H]2CCN(C3CCNCC3)C2=O)c(F)c1]

Step A:

A solution of HBTU (8.1 g, 21 mmol), (S)-2-(tert-butoxycarbonylamino)-4-(methylthio)butanoic acid (5.3 g, 21 mmol) and DIEA (8.2 mL, 47 mmol) in DMF (50 mL) was stirred at ambient temperature for 30 minutes. Benzyl 4-aminopiperidine-1-carboxylate (5.0 g, 21 mmol) was added and the mixture was stirred at ambient temperature for 18 hours. The mixture was poured into 1N NaOH (500 mL) and extracted into EtOAc (500 mL). The combined organic layers were washed with 1N HCl (500 mL) and brine (500 mL), dried over MgSO₄, filtered and concentrated in vacuo to yield (S)-benzyl 4-(3-(2-fluoro-4-(methylsulfonyl)phenylamino)-2-oxopyrrolidin-1-yl)piperidine-1-carboxylate (10 g, 21 mmol, 100%).

Step B:

A solution of (S)-benzyl-4-(2-(tert-butoxycarbonylamino)-4-(methylthio)butanamido)piperidine-1-carboxylate (10 g, 21.5 mmol) in neat MeI (40.2 mL, 640 mmol) was stirred at ambient temperature for 4 hours. The reaction was evaporated to dryness to yield (S)-benzyl-4-(3-(2-fluoro-4-(methylsulfonyl)phenylamino)-2-oxopyrrolidin-1-yl)piperidine-1-carboxylate methiodide salt (10 g, 17 mmol, 79%).

Step C:

(S)-bBenzyl-4-(3-(2-fluoro-4-(methylsulfonyl)phenylamino)-2-oxopyrrolidin-1-yl)piperidine-1-carboxylate methiodide salt (10 g, 17 mmol) was dissolved in dry THF (100 mL) and cooled to 0° C. Lithium bis(trimethylsilyl)amide (21 mL, 21 mmol) was added and the mixture was warmed to ambient temperature and stirred for 2 hours. The mixture was poured into saturated ammonium chloride (100 mL) and extracted into EtOAc (3×100 mL). The combined organic layers were washed with brine, dried over MgSO₄ and concentrated in vacuo to yield (S)-benzyl 4-(3-(tert-butoxycarbonylamino)-2-oxopyrrolidin-1-yl)piperidine-1-carboxylate (7 g, 17 mmol, 100%).

Step D:

A solution of (S)-benzyl-4-(3-(tert-butoxycarbonylamino)-2-oxopyrrolidin-1-yl)piperidine-1-carboxylate (7 g, 17 mmol) in 50% TFA/CH₂Cl₂ (50 mL) was stirred at ambient temperature for 1 hour. The mixture was concentrated in vacuo. The residue was dissolved in EtOAc (200 mL) and washed with saturated sodium carbonate (200 mL) and brine. The combined organic layers were dried over MgSO₄ and concentrated in vacuo to yield (S)-benzyl 4-(3-amino-2-oxopyrrolidin-1-yl)piperidine-1-carboxylate (3.4 g, 11 mmol, 64%).

Step E:

A solution of (S)-benzyl 4-(3-amino-2-oxopyrrolidin-1-yl)piperidine-1-carboxylate (2.0 g, 6.3 mmol), 1,2-difluoro-4-(methylsulfonyl)benzene (1.2 g, 6.3 mmol), and Na₂CO₃ (3.3 g, 32 mmol) in DMSO (20 mL) was stirred at 120° C. for 48 hours. The reaction mixture was poured into water (200 mL) and extracted with EtOAc (3×100 mL). The combined organic layers were washed with brine, dried over MgSO₄, filtered and concentrated in vacuo. The material was purified over silica gel (100% EtOAc) to yield (S)-benzyl 4-(3-(2-fluoro-4-(methylsulfonyl)phenylamino)-2-oxopyrrolidin-1-yl)piperidine-1-carboxylate (1.3 g, 2.7 mmol, 42%).

Step F:

A solution of (S)-benzyl-4-(3-(2-fluoro-4-(methylsulfonyl)phenylamino)-2-oxopyrrolidin-1-yl)piperidine-1-carboxylate (1.3 g, 27 mmol) in ethanol (20 mL) and concentrated HCl (300 μL) was hydrogenated at 40 PSI with 10% Degussa type Pd/C (650 mg) for 18 hours. The mixture was filtered through Celite® and the solids were washed with MeOH (200 mL) and water (200 mL). The methanol in the filtrate was removed in vacuo. The aqueous layer was extracted with EtOAc (3×100 mL). The combined organic layers were washed with brine, dried over MgSO₄, filtered and concentrated in vacuo to yield (S)-3-(2-fluoro-4-(methylsulfonyl)phenylamino)-1-(piperidin-4-yl)pyrrolidin-2-one (600 mg, 1.7 mmol, 64%). Mass spectrum (apci) m/z=356.1 (M+H).

The following compounds were also prepared according to the procedure above.

| | | | Mass spectrum |
|---|---|---|---|
| Preparation | Structure | Name | (apci) |
| GG | 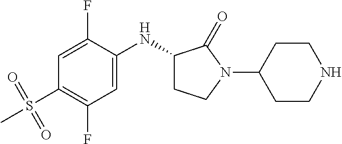 [SMILES: CS(=O)(=O)c1cc(F)c(N[C@H]2CCN(C3CCNCC3)C2=O)cc1F] | (S)-3-(2,5-difluoro-4- (methylsulfonyl)phenyl- amino)-1-(piperidin-4- yl)pyrrolidin-2-one | m/z = 374.2 (M + H) |
| HH | 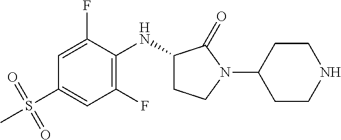 [SMILES: CS(=O)(=O)c1cc(F)c(N[C@H]2CCN(C3CCNCC3)C2=O)c(F)c1] | (S)-3-(2,6-difluoro-4- (methylsulfonyl)phenyl- amino)-1-(piperidin-4- yl)pyrrolidin-2-one | m/z = 374.2 (M + H) |

### Preparation II

### (S)-3-(2-fluoro-4-(methylsulfonyl)phenoxy)-1-(piperidin-4-yl)pyrrolidin-2-one hydrochloride

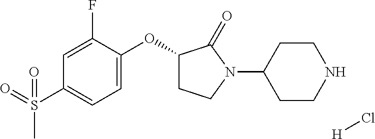

Step A:

To a solution of (R)-tert-butyl 4-(3-(methylsulfonyloxy)-2-oxopyrrolidin-1-yl)piperidine-1-carboxylate (1.7 g, 4.7 mmol) in dry DMSO (30 mL) was added 4-bromo-2-fluorophenol (1.1 g, 5.6 mmol) and K₂CO₃ (0.78 g, 5.6 mmol). The reaction was heated to 70° C. under nitrogen for 3 hr. The reaction was poured into water and extracted with EtOAc (3×50 mL), washed with brine, dried over Na₂SO₄, filtered and concentrated in vacuo. The residue was purified over silica gel (40% EtOAc in hexanes) to afford (S)-tert-butyl 4-(3-(4-bromo-2-fluorophenoxy)-2-oxopyrrolidin-1-yl)piperidine-1-carboxylate (1.8 g, 3.9 mmol, 84% yield) as a white solid.

Step B:

(S)-tert-butyl 4-(3-(4-bromo-2-fluorophenoxy)-2-oxopyrrolidin-1-yl)piperidine-1-carboxylate (1.8 g, 3.9 mmol) was dissolved in DMSO (30 mL) and purged with nitrogen. Sodium methanesulfinate (0.60 g, 5.9 mmol) and trans-cyclohexane-1,2-diamine (0.19 mL, 1.6 mmol) were added followed by Cu(I) Triflate benzene complex (0.20 g, 0.39 mmol). The reaction was placed in a 110° C. oil bath under nitrogen and stirred overnight. The reaction was cooled to ambient temperature, partitioned between water and EtOAc and extracted with EtOAc (2×50 mL). The combined organic layers were washed with brine, dried over Na₂SO₄, filtered and concentrated. The residue was purified over silica gel (100% EtOAc) to afford (S)-tert-butyl 4-(3-(2-fluoro-4-(methylsulfonyl)phenoxy)-2-oxopyrrolidin-1-yl)piperidine-1-carboxylate (1.6 g, 3.5 mmol, 89% yield) as a white solid.

Step C:

(S)-tert-butyl 4-(3-(2-fluoro-4-(methylsulfonyl)phenoxy)-2-oxopyrrolidin-1-yl)piperidine-1-carboxylate (1.6 g, 3.5 mmol) was dissolved in CH₂Cl₂ (20 mL) and 4N HCl in dioxane (15 mL) was added and the mixture was stirred at ambient temperature overnight. The reaction was concentrated in vacuo to afford (S)-3-(2-fluoro-4-(methylsulfonyl)phenoxy)-1-(piperidin-4-yl)pyrrolidin-2-one hydrochloride (1.5 g, 3.8 mmol, 109% yield) as a white solid. Mass spectrum (apci) m/z=357.2 (M+H).

The following compounds were prepared according to the procedure above.

| | | | Mass spectrum |
|---|---|---|---|
| Preparation | Structure | Name | (apci) |
| JJ | 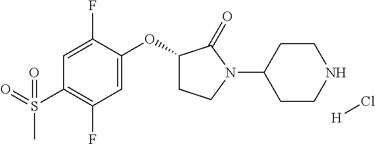 | (S)-3-(2,5-difluoro-4- (methylsulfonyl)phenoxy)- 1-(piperidin-4-yl)pyrrolidin- 2-one hydrochloride | m/z = 375.1 (M + H) |
| KK | 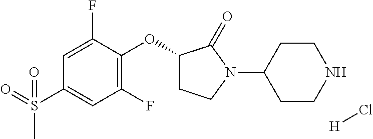 | (S)-3-(2,6-difluoro-4- (methylsulfonyl)phenoxy)- 1-(piperidin-4-yl)pyrrolidin- 2-one hydrochloride | m/z = 375.1 (M + H) |

### Preparation LL

### (S)-tert-butyl 3-(2-fluoro-4-(methylsulfonyl)phenylamino)-2-oxo-1,4′-bipiperidine-1′-carboxylate

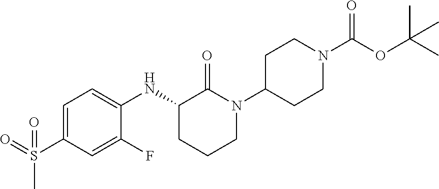 [SMILES: CC(C)(C)OC(=O)N1CCC(N2CCC[C@H](Nc3ccc(S(C)(=O)=O)cc3F)C2=O)CC1]

Step A:

(S)-5-Amino-2-(benzyloxycarbonylamino)pentanoic acid (5.0 g, 19 mmol) was dissolved in THF (100 mL). Water (20 mL) and tert-butyl 4-oxopiperidine-1-carboxylate (3.7 g, 19 mmol) were added and the mixture was stirred at ambient temperature for 1 hour. The reaction was cooled to 0° C. and 1.0 M NaCNBH₃ (19 mL, 19 mmol) was added. The mixture was allowed to stir at ambient temperature overnight. The solvent was removed in vacuo to provide crude (S)-2-(benzyloxycarbonylamino)-5-(1-(tert-butoxycarbonyl)piperidin-4-ylamino)pentanoic acid (8.4 g, 19 mmol, 100% yield) which was taken forward without further purification.

Step B:

Crude (S)-2-(benzyloxycarbonylamino)-5-(1-(tert-butoxycarbonyl)piperidin-4-ylamino)pentanoic acid (8.4 g, 18.7 mmol) was dissolved in DMF (100 mL) and cooled to 0° C. EDCI (3.58 g, 18.7 mmol) and N-ethyl-N-isopropylpropan-2-amine (3.25 mL, 18.7 mmol) were added and the reaction was allowed to warm to ambient temperature overnight. The reaction was diluted with EtOAc and washed with 1N HCl, saturated aqueous NaHCO₃ and brine. The organic layer was dried over Na₂SO₄, filtered and concentrated. The residue was purified over silica gel (50 to 80% EtOAc in hexanes) to afford (S)-tert-butyl 3-(benzyloxycarbonylamino)-2-oxo-1,4′-bipiperidine-1′-carboxylate (5.2 g, 12.1 mmol, 64.5% yield).

Step C:

(S)-tert-butyl 3-(benzyloxycarbonylamino)-2-oxo-1,4′-bipiperidine-1′-carboxylate (5.2 g, 12 mmol) was dissolved in methanol (100 mL) and 10% Pd/C was added and stirred under balloon pressure of hydrogen for 3 hr. The reaction was filtered through celite and concentrated to afford (S)-tert-butyl 3-amino-2-oxo-1,4′-bipiperidine-1′-carboxylate (4.2 g, 14 mmol, 117% yield) as a pale yellow oil.

Step D:

(S)-tert-butyl 3-amino-2-oxo-1,4′-bipiperidine-1′-carboxylate (1.0 g, 3.36 mmol) was dissolved in DMSO (20 mL). 1,2-difluoro-4-(methylsulfonyl)benzene (0.775 g, 4.04 mmol) and Na₂CO₃ (0.535 g, 5.04 mmol) were added and the reaction was heated to 120° C. under nitrogen overnight. The reaction was cooled to ambient temperature, water added and extracted with EtOAc, dried over Na₂SO₄, filtered and concentrated. The residue was purified over silica gel (80% EtOAc in hexanes) to afford (S)-tert-butyl 3-(2-fluoro-4-(methylsulfonyl)phenylamino)-2-oxo-1,4′-bipiperidine-1′-carboxylate (680 mg, 1.45 mmol, 43.1% yield) as a white solid. Mass spectrum (apci) m/z=370.2 (M+H-Boc).

The following compound was prepared according to the procedure of Preparation LL.

| | | | Mass spectrum |
|---|---|---|---|
| Preparation | Structure | Name | (apci) |
| MM | 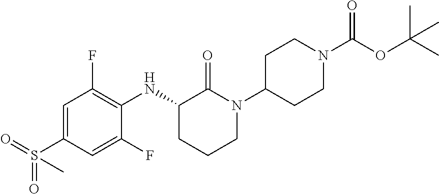 [SMILES: CC(C)(C)OC(=O)N1CCC(N2CCC[C@H](Nc3c(F)cc(S(C)(=O)=O)cc3F)C2=O)CC1] | (S)-tert-butyl 3-(2,6-difluoro- 4-(methylsulfonyl) phenylamino)-2-oxo-1,4′- bipiperidine-1′-carboxylate | m/z = 388.2 (M + H-Boc) |

### Example 1

### (S)-3-(2-fluoro-4-(methylsulfonyl)phenylamino)-1-(1-(3-isopropyl-1,2,4-thiadiazol-5-yl)piperidin-4-yl)pyrrolidin-2-one

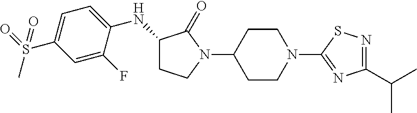 [SMILES: CC(C)c1nsc(N2CCC(N3CC[C@H](Nc4ccc(S(C)(=O)=O)cc4F)C3=O)CC2)n1]

Step A:

(S)-2-(tert-Butoxycarbonylamino)-4-(methylthio)butanoic acid (0.949 g, 3.81 mmol) was dissolved in DMF (20 mL). N-ethyl-N-isopropylpropan-2-amine (1.99 mL, 11.4 mmol), 1H-benzo[d][1,2,3]triazol-1-ol (0.0514 g, 0.381 mmol) and EDCI (0.875 g, 4.57 mmol) were added and the mixture was stirred at ambient temperature. 1-(3-Isopropyl-1,2,4-thiadiazol-5-yl)piperidin-4-amine hydrochloride (1.0 g, 3.81 mmol) was added and the reaction was stirred at ambient temperature for 2 days. The reaction was poured into water (150 mL) and stirred for 20 minutes. The aqueous layer was decanted and the solids were dissolved in EtOAc. The combined organic layers were washed with brine, dried over Na₂SO₄, filtered and concentrated in vacuo to afford (S)-tert-butyl 1-(1-(3-isopropyl-1,2,4-thiadiazol-5-yl)piperidin-4-ylamino)-4-(methylthio)-1-oxobutan-2-ylcarbamate (1.4 g, 3.06 mmol, 80.4% yield) as a white solid.

Step B:

(S)-tert-Butyl 1-(1-(3-isopropyl-1,2,4-thiadiazol-5-yl)piperidin-4-ylamino)-4-(methylthio)-1-oxobutan-2-ylcarbamate (1.4 g, 3.059 mmol) was dissolved in iodomethane (7.6 mL, 122 mmol) and the reaction was stirred at ambient temperature overnight. The methyl iodide was removed in vacuo to afford (S)-(3-(tert-butoxycarbonylamino)-4-(1-(3-isopropyl-1,2,4-thiadiazol-5-yl)piperidin-4-ylamino)-4-oxobutyl)dimethylsulfonium iodide (1.8 g, 3.0 mmol, 98% yield) as a yellow solid.

Step C:

(S)-(3-(tert-Butoxycarbonylamino)-4-(1-(3-isopropyl-1,2,4-thiadiazol-5-yl)piperidin-4-ylamino)-4-oxobutyl)dimethylsulfonium iodide (1.8 g, 3.0 mmol) was dissolved in THF (30 mL) and cooled to 0° C. 1M LHMDS (3.0 mL, 3.0 mmol) was added and the reaction was stirred at 0° C. for 3 hours and then warmed to ambient temperature and stirred for 2 hours. The reaction was partitioned between water and EtOAc, dried over Na₂SO₄, filtered and concentrated in vacuo. The residue was purified over silica gel (80 to 100% EtOAc in hexanes) to afford (S)-tert-butyl 1-(1-(3-isopropyl-1,2,4-thiadiazol-5-yl)piperidin-4-yl)-2-oxopyrrolidin-3-ylcarbamate (1.1 g, 2.7 mmol, 89% yield).

Step D:

(S)-tert-Butyl 1-(1-(3-isopropyl-1,2,4-thiadiazol-5-yl)piperidin-4-yl)-2-oxopyrrolidin-3-ylcarbamate (8.4 g, 21 mmol) was dissolved in CH₂Cl₂ (100 mL). TFA (30 mL) was added and the mixture was stirred at ambient temperature for 1 hour. The reaction was concentrated in vacuo, partitioned between saturated aqueous NaHCO₃ and EtOAc and extracted with EtOAc. The combined organic layers were dried over Na₂SO₄, filtered and concentrated to afford (S)-3-amino-1-(1-(3-isopropyl-1,2,4-thiadiazol-5-yl)piperidin-4-yl)pyrrolidin-2-one (5.6 g, 18 mmol, 88% yield).

Step E:

(S)-3-Amino-1-(1-(3-isopropyl-1,2,4-thiadiazol-5-yl)piperidin-4-yl)pyrrolidin-2-one (500 mg, 1.62 mmol) was dissolved in DMSO (8 mL) and 1,2-difluoro-4-(methylsulfonyl)benzene (621 mg, 3.23 mmol) and Na₂CO₃ (171 mg, 1.62 mmol) were added. The reaction was heated to 120° C. for 3 days. The reaction was partitioned between water and EtOAc and extracted with EtOAc. The combined organic layers were dried over Na₂SO₄, filtered and concentrated. The residue was purified over silica gel (0 to 5% methanol in EtOAc) to afford (S)-3-(2-fluoro-4-(methylsulfonyl)phenylamino)-1-(1-(3-isopropyl-1,2,4-thiadiazol-5-yl)piperidin-4-yl)pyrrolidin-2-one (420 mg, 0.872 mmol, 54.0% yield) as a tan solid. Mass spectrum (apci) m/z=482.2 (M+H).

The following compounds were also prepared according to the procedure described for Example 1.

| | | | Mass spectrum |
|---|---|---|---|
| Example | Structure | Name | (apci) |
| 2 | 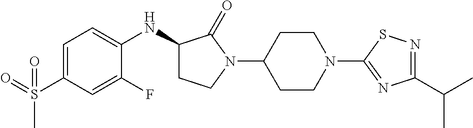 [SMILES: CC(C)c1nsc(N2CCC(N3CC[C@@H](Nc4ccc(S(C)(=O)=O)cc4F)C3=O)CC2)n1] | (R)-3-(2-fluoro-4- (methylsulfonyl)phenylamino)- 1-(1-(3-isopropyl-1,2,4- thiadiazol-5-yl)piperidin-4- yl)pyrrolidin-2-one | m/z = 482.3 (M + H). |
| 3 | 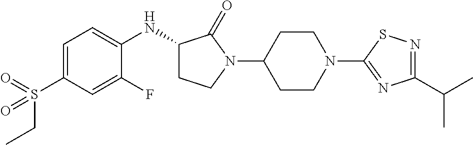 [SMILES: CCS(=O)(=O)c1ccc(N[C@H]2CCN(C3CCN(c4nc(C(C)C)ns4)CC3)C2=O)c(F)c1] | (S)-3-(4-(ethylsulfonyl)-2- fluorophenylamino)-1-(1-(3- isopropyl-1,2,4-thiadiazol-5- yl)piperidin-4-yl)pyrrolidin-2- one | m/z = 496.2 (M + H). |
| 4 | 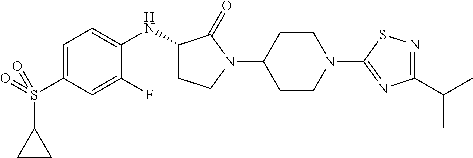 [SMILES: CC(C)c1nsc(N2CCC(N3CC[C@H](Nc4ccc(S(=O)(=O)C5CC5)cc4F)C3=O)CC2)n1] | (S)-3-(4-(cyclopropylsulfonyl)- 2-fluorophenylamino)-1-(1-(3- isopropyl-1,2,4-thiadiazol-5- yl)piperidin-4-yl)pyrrolidin-2- one | m/z = 508.2 (M + H). |
| 5 | 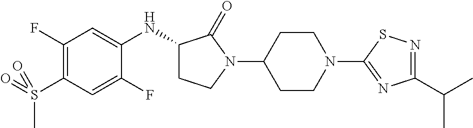 [SMILES: CC(C)c1nsc(N2CCC(N3CC[C@H](Nc4cc(F)c(S(C)(=O)=O)cc4F)C3=O)CC2)n1] | (S)-3-(2,5-difluoro-4- (methylsulfonyl)phenylamino)- 1-(1-(3-isopropyl-1,2,4- thiadiazol-5-yl)piperidin-4- yl)pyrrolidin-2-one | m/z = 500.3 (M + H). |
| 6 | 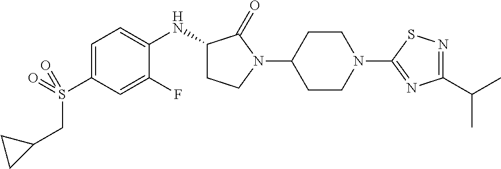 [SMILES: CC(C)c1nsc(N2CCC(N3CC[C@H](Nc4ccc(S(=O)(=O)CC5CC5)cc4F)C3=O)CC2)n1] | (S)-3-(4- (cyclopropylmethylsulfonyl)- 2-fluorophenylamino)-1-(1-(3- isopropyl-1,2,4-thiadiazol-5- yl)piperidin-4-yl)pyrrolidin-2- one | m/z = 522.2 (M + H). |
| 7 | 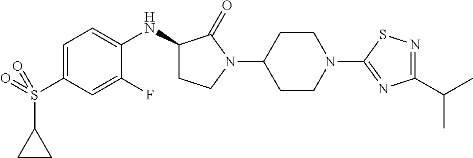 [SMILES: CC(C)c1nsc(N2CCC(N3CC[C@@H](Nc4ccc(S(=O)(=O)C5CC5)cc4F)C3=O)CC2)n1] | (R)-3-(4- (cyclopropylsulfonyl)-2- fluorophenylamino)-1-(1-(3- isopropyl-1,2,4-thiadiazol-5- yl)piperidin-4-yl)pyrrolidin-2- one | m/z = 508.3 (M + H). |
| 8 | 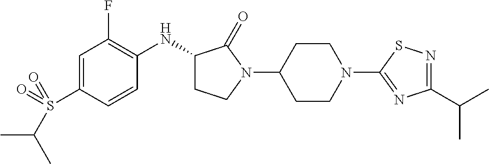 [SMILES: CC(C)c1nsc(N2CCC(N3CC[C@H](Nc4ccc(S(=O)(=O)C(C)C)cc4F)C3=O)CC2)n1] | (S)-3-(2-fluoro-4- (isopropylsulfonyl)phenylamino)- 1-(1-(3-isopropyl-1,2,4- thiadiazol-5-yl)piperidin-4- yl)pyrrolidin-2-one | m/z = 510.2 (M + H). |
| 9 | 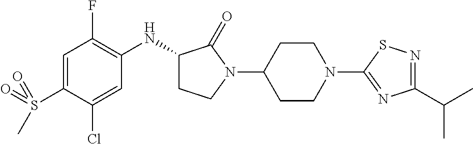 [SMILES: CC(C)c1nsc(N2CCC(N3CC[C@H](Nc4cc(Cl)c(S(C)(=O)=O)cc4F)C3=O)CC2)n1] | (S)-3-(5-chloro-2-fluoro-4- (methylsulfonyl)phenylamino)- 1-(1-(3-isopropyl-1,2,4- thiadiazol-5-yl)piperidin-4- yl)pyrrolidin-2-one | m/z = 516.2 (M + H). |
| 10 | 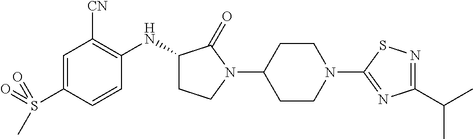 [SMILES: CC(C)c1nsc(N2CCC(N3CC[C@H](Nc4ccc(S(C)(=O)=O)cc4C#N)C3=O)CC2)n1] | (S)-2-(1-(1-(3-isopropyl-1,2,4- thiadiazol-5-yl)piperidin-4-yl)- 2-oxopyrrolidin-3-ylamino)-5- (methylsulfonyl)benzonitrile | m/z = 489.2 (M + H). |
| 11 | 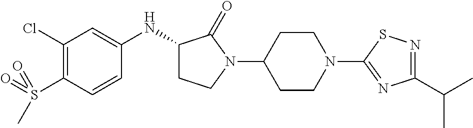 [SMILES: CC(C)c1nsc(N2CCC(N3CC[C@H](Nc4ccc(S(C)(=O)=O)c(Cl)c4)C3=O)CC2)n1] | (S)-3-(3-chloro-4- (methylsulfonyl)phenylamino)- 1-(1-(3-isopropyl-1,2,4- thiadiazol-5-yl)piperidin-4- yl)pyrrolidin-2-one | m/z = 498.2 (M + H). |
| 12 | 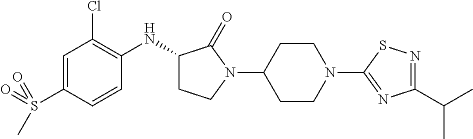 [SMILES: CC(C)c1nsc(N2CCC(N3CC[C@H](Nc4ccc(S(C)(=O)=O)cc4Cl)C3=O)CC2)n1] | (S)-3-(2-chloro-4- (methylsulfonyl)phenylamino)- 1-(1-(3-isopropyl-1,2,4- thiadiazol-5-yl)piperidin-4- yl)pyrrolidin-2-one | m/z = 498.2 (M + H). |
| 13 | 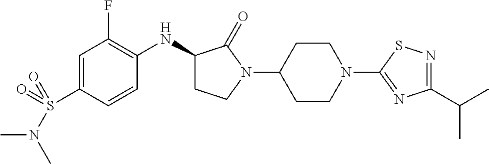 [SMILES: CC(C)c1nsc(N2CCC(N3CC[C@@H](Nc4ccc(S(=O)(=O)N(C)C)cc4F)C3=O)CC2)n1] | (R)-3-fluoro-4-(1-(1-(3- isopropyl-1,2,4-thiadiazol-5- yl)piperidin-4-yl)-2- oxopyrrolidin-3-ylamino)-N,N- dimethylbenzenesulfonamide | m/z = 511.2 (M + H). |

### Example 14

### (S)-1-(1-(3-isopropyl-1,2,4-thiadiazol-5-yl)piperidin-4-yl)-3-(5-(methylsulfonyl)pyrimidin-2-ylamino)pyrrolidin-2-one

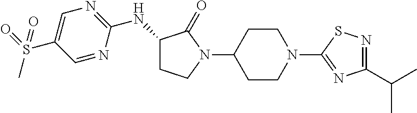 [SMILES: CC(C)c1nsc(N2CCC(N3CC[C@H](Nc4ncc(S(C)(=O)=O)cn4)C3=O)CC2)n1]

Step A:

(S)-3-Amino-1-(1-(3-isopropyl-1,2,4-thiadiazol-5-yl)piperidin-4-yl)pyrrolidin-2-one (Example 1; Steps A-D; 100 mg, 0.323 mmol) was dissolved in THF (3 mL) and N-ethyl-N-isopropylpropan-2-amine (84.4 μL, 0.485 mmol) and 5-bromo-2-chloropyrimidine (62.5 mg, 0.323 mmol) were added. The reaction was heated to 60° C. for 2 days. The reaction was partitioned between water and EtOAc, dried over Na₂SO₄, filtered and concentrated. The residue was purified over silica gel (0 to 10% methanol in EtOAc) to afford (S)-3-(5-bromopyrimidin-2-ylamino)-1-(1-(3-isopropyl-1,2,4-thiadiazol-5-yl)piperidin-4-yl)pyrrolidin-2-one (48.3 mg, 0.104 mmol, 32.0% yield).

Step B:

(S)-3-(5-Bromopyrimidin-2-ylamino)-1-(1-(3-isopropyl-1,2,4-thiadiazol-5-yl)piperidin-4-yl)pyrrolidin-2-one (48 mg, 0.103 mmol) was dissolved in DMSO (2 mL) and nitrogen was bubbled through the reaction mixture for 15 minutes. Sodium methanesulfinate (15.8 mg, 0.154 mmol), trans-cyclohexane-1,2-diamine (4.95 μL, 0.0412 mmol) and Cu(I) triflate benzene complex (5.18 mg, 0.0103 mmol) were added. The reaction flask was placed into a 110° C. oil bath and the reaction mixture was stirred under nitrogen overnight. The reaction mixture was partitioned between water and EtOAc and extracted with EtOAc. The combined organic layers were dried over Na₂SO₄, filtered and concentrated. The residue was purified over silica gel (4% methanol in EtOAc) to afford (S)-1-(1-(3-isopropyl-1,2,4-thiadiazol-5-yl)piperidin-4-yl)-3-(5-(methylsulfonyl)pyrimidin-2-ylamino)pyrrolidin-2-one (29.6 mg, 0.0636 mmol, 61.8% yield) as a white solid. Mass spectrum (apci) m/z=466.2 (M+H).

### Example 15

### (S)-3-(5-Bromopyridin-2-ylamino)-1-(1-(3-isopropyl-1,2,4-thiadiazol-5-yl)piperidin-4-yl)pyrrolidin-2-one

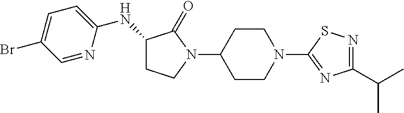 [SMILES: CC(C)c1nsc(N2CCC(N3CC[C@H](Nc4ccc(Br)cn4)C3=O)CC2)n1]

Step A:

(S)-3-Amino-1-(1-(3-isopropyl-1,2,4-thiadiazol-5-yl)piperidin-4-yl)pyrrolidin-2-one (Example 1; Steps A-D; 350 mg, 1.13 mmol) was dissolved in Toluene (10 mL). Xantphos (98.2 mg, 0.170 mmol), 2,5-dibromopyridine (295 mg, 1.24 mmol) and sodium 2-methylpropan-2-olate (163 mg, 1.70 mmol) were added and nitrogen bubbled through the mixture for 5 minutes. Pd₂dba₃ (51.8 mg, 0.0566 mmol) was added and the reaction was placed in a 100° C. oil bath and stirred at 100° C. overnight. The reaction was cooled to ambient temperature, partitioned between EtOAc and water, dried over Na₂SO₄, filtered and concentrated. The residue was purified over silica gel (70 to 100% EtOAc in hexanes) to afford (S)-3-(5-bromopyridin-2-ylamino)-1-(1-(3-isopropyl-1,2,4-thiadiazol-5-yl)piperidin-4-yl)pyrrolidin-2-one (137 mg, 0.294 mmol, 26.0% yield) as a tan solid. Mass spectrum (apci) m/z=465.2, 467.1 (M+H).

### Example 16

### (S)-1-(1-(3-isopropyl-1,2,4-thiadiazol-5-yl)piperidin-4-yl)-3-(5-(methylsulfonyl)pyridin-2-ylamino)pyrrolidin-2-one

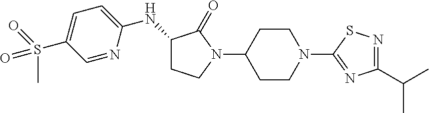 [SMILES: CC(C)c1nsc(N2CCC(N3CC[C@H](Nc4ccc(S(C)(=O)=O)cn4)C3=O)CC2)n1]

Step A:

(S)-3-(5-bromopyridin-2-ylamino)-1-(1-(3-isopropyl-1,2,4-thiadiazol-5-yl)piperidin-4-yl)pyrrolidin-2-one (Example 15; 130 mg, 0.279 mmol) was dissolved in DMSO (3 mL) and nitrogen was bubbled through the mixture for 5 minutes. Sodium methanesulfinate (50.3 mg, 0.419 mmol), trans-cyclohexane-1,2-diamine (13.4 μl, 0.112 mmol) and Cu(I) Triflate benzene complex (14.1 mg, 0.0279 mmol) were added and the reaction was placed in a 110° C. oil bath and stirred under nitrogen overnight. The reaction was cooled to ambient temperature and water added and extracted with EtOAc, dried over Na₂SO₄, filtered and concentrated. The residue was purified over silica gel (5% methanol in EtOAc) twice to afford (S)-1-(1-(3-isopropyl-1,2,4-thiadiazol-5-yl)piperidin-4-yl)-3-(5-(methylsulfonyl)pyridin-2-ylamino)pyrrolidin-2-one (84 mg, 0.181 mmol, 64.7% yield) as a tan solid. Mass spectrum (apci) m/z=465.2 (M+H).

The following compound was prepared according to the procedure described for Example 16.

| | | | Mass spectrum |
|---|---|---|---|
| Example | Structure | Name | (apci) |
| 17 | 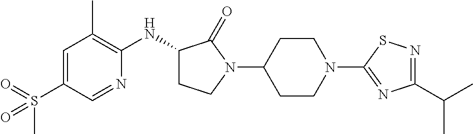 [SMILES: Cc1cc(S(C)(=O)=O)cnc1N[C@H]1CCN(C2CCN(c3nc(C(C)C)ns3)CC2)C1=O] | (S)-1-(1-(3-isopropyl-1,2,4- thiadiazol-5-yl)piperidin-4- yl)-3-(3-methyl-5- (methylsulfonyl)pyridin-2- ylamino)pyrrolidin-2-one | m/z = 479.2 (M + H). |

### Example 18

### (S)-3-(2-fluoro-5-methyl-4-(methylsulfonyl)phenylamino)-1-(1-(3-isopropyl-1,2,4-thiadiazol-5-yl)piperidin-4-yl)pyrrolidin-2-one

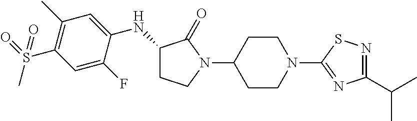 [SMILES: Cc1cc(N[C@H]2CCN(C3CCN(c4nc(C(C)C)ns4)CC3)C2=O)c(F)cc1S(C)(=O)=O]

Step A:

(S)-Benzyl 4-(3-amino-2-oxopyrrolidin-1-yl)piperidine-1-carboxylate (Preparation FF, Steps A-D; 750 mg, 2.36 mmol) was dissolved in toluene (20 mL). Racemic Binap (147 mg, 0.236 mmol), 1-bromo-2-fluoro-5-methyl-4-(methylsulfonyl)benzene (947 mg, 3.54 mmol) and Cs₂CO₃ (924 mg, 2.84 mmol) were added and the reaction was bubbled through with nitrogen for 5 minutes. Pd₂ dba₃ (108 mg, 0.118 mmol) was added and the reaction was placed in a 95° C. oil bath overnight. The reaction was cooled to ambient temperature, filtered and concentrated. The residue was purified over silica gel (50 to 100% EtOAc in hexanes) to afford (S)-benzyl 4-(3-(2-fluoro-5-methyl-4-(methylsulfonyl)phenylamino)-2-oxopyrrolidin-1-yl)piperidine-1-carboxylate (906 mg, 1.80 mmol, 76.1% yield) as a tan solid.

Step B:

(S)-Benzyl 4-(3-(2-fluoro-5-methyl-4-(methylsulfonyl)phenylamino)-2-oxopyrrolidin-1-yl)piperidine-1-carboxylate (906 mg, 1.80 mmol) was dissolved in EtOH (15 mL) and 10% Pd/C was added and the mixture was stirred under balloon pressure of hydrogen overnight. The reaction was filtered and concentrated to afford (S)-3-(2-fluoro-5-methyl-4-(methylsulfonyl)phenylamino)-1-(piperidin-4-yl)pyrrolidin-2-one (680 mg, 1.84 mmol, 102% yield).

Step C:

N-(Methylsulfonyloxy)isobutyrimidoyl chloride (169 mg, 0.844 mmol) was dissolved in CH₃CN (7 mL). Pyridine (228 μL, 2.81 mmol) and NaNCS (68.5 mg, 0.844 mmol) were added and the reaction was heated to 45° C. for 45 minutes. (S)-3-(2-fluoro-5-methyl-4-(methylsulfonyl)phenylamino)-1-(piperidin-4-yl)pyrrolidin-2-one (260 mg, 0.704 mmol) was added and the reaction was heated at 45° C. overnight. The reaction was partitioned between water and EtOAc, dried over Na₂SO₄, filtered and concentrated. The residue was purified over silica gel (70 to 100% EtOAc in hexanes) to afford (S)-3-(2-fluoro-5-methyl-4-(methylsulfonyl)phenylamino)-1-(1-(3-isopropyl-1,2,4-thiadiazol-5-yl)piperidin-4-yl)pyrrolidin-2-one (140 mg, 0.282 mmol, 40.1% yield) as a white solid. Mass spectrum (apci) m/z=496.3.

### Example 19

### 3-(4-(Ethylsulfonyl)-3-methylphenylamino)-1-(1-(3-isopropyl-1,2,4-thiadiazol-5-yl)piperidin-4-yl)pyrrolidin-2-one

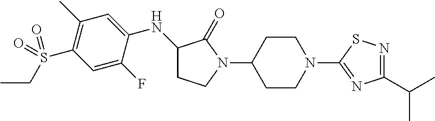 [SMILES: CCS(=O)(=O)c1cc(F)c(NC2CCN(C3CCN(c4nc(C(C)C)ns4)CC3)C2=O)cc1C]

To a solution of (S)-3-amino-1-(1-(3-isopropyl-1,2,4-thiadiazol-5-yl)piperidin-4-yl)pyrrolidin-2-one (Example 1; Steps A-D; 0.1 g, 0.32 mmol) in toluene (10 mL) continuously purged with nitrogen was added 4-bromo-1-(ethylsulfonyl)-2-methylbenzene (0.17 g, 0.65 mmol), Pd₂ dba₃ (0.015 g, 0.016 mmol), racemic 2,2′-bis(diphenylphosphino)-1,1′-binaphthyl (BINAP) (0.020 g, 0.032 mmol) and sodium 2-methylpropan-2-olate (0.037 g, 0.39 mmol) and the reaction was heated to 80° C. overnight. The reaction was poured into EtOAc and washed with water, brine, dried over MgSO₄, filtered and concentrated in vacuo. The crude material was purified over silica gel (1.5% methanol/CH₂Cl₂) to afford 3-(4-(ethylsulfonyl)-3-methylphenylamino)-1-(1-(3-isopropyl-1,2,4-thiadiazol-5-yl)piperidin-4-yl)pyrrolidin-2-one (0.027 g, 0.055 mmol, 17% yield). Mass Spectrum (apci) m/z=492.3 (M+H).

The following compounds were also prepared according to the procedure described for Example 19.

| | | | Mass spectrum |
|---|---|---|---|
| Example | Structure | Name | (apci) |
| 20 | 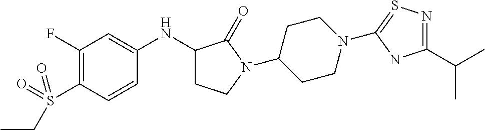 | 3-(4-(ethylsulfonyl)-3- fluorophenylamino)-1-(1-(3- isopropyl-1,2,4-thiadiazol-5- yl)piperidin-4-yl)pyrrolidin- 2-one | m/z = 496.3 (M + H). |
| 21 | 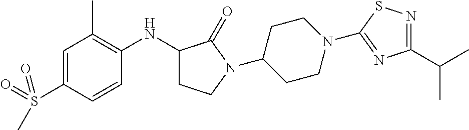 [SMILES: Cc1cc(S(C)(=O)=O)ccc1NC1CCN(C2CCN(c3nc(C(C)C)ns3)CC2)C1=O] | 1-(1-(3-isopropyl-1,2,4- thiadiazol-5-yl)piperidin-4- yl)-3-(2-methyl-4-(methyl- sulfonyl)phenylamino) pyrrolidin-2-one | m/z = 478.2 (M + H). |

### Example 22

### (S)-3-(2-fluoro-4-(methylsulfonyl)phenylamino)-1-(1-(3-isobutyl-1,2,4-thiadiazol-5-yl)piperidin-4-yl)pyrrolidin-2-one trifluoroacetate

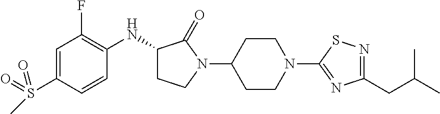 [SMILES: CC(C)Cc1nsc(N2CCC(N3CC[C@H](Nc4ccc(S(C)(=O)=O)cc4F)C3=O)CC2)n1]

To a solution of (E)-3-methyl-N-(methylsulfonyloxy)butanimidoyl chloride (0.0661 g, 0.309 mmol) in EtOAc (10 mL) was added pyridine (0.089 g, 1.1 mmol) and sodium isothiocyanate (0.027 g, 0.34 mmol). The reaction was heated to 60° C. for 1 hour. (S)-3-(2-Fluoro-4-(methylsulfonyl)phenylamino)-1-(piperidin-4-yl)pyrrolidin-2-one (Preparation FF; 0.10 g, 0.28 mmol) was added and the reaction was stirred overnight at 60° C. The reaction was diluted with EtOAc, washed with 1N sodium hydroxide and brine, dried over MgSO₄, filtered and concentrated in vacuo. The crude material was purified over silica gel (1:1 EtOAc/CH₂Cl₂ to 100% EtOAc). The isolated solid was purified on reverse preparative HPLC (5 to 95% acetonitrile in water with 0.1% TFA) to yield (S)-3-(2-fluoro-4-(methylsulfonyl)phenylamino)-1-(1-(3-isobutyl-1,2,4-thiadiazol-5-yl)piperidin-4-yl)pyrrolidin-2-one trifluoroacetate (0.0135 g, 0.0272 mmol, 9.68% yield). Mass Spectrum (apci) m/z=496.2 (M+H).

The following compounds were also prepared according to the procedure of Example 22.

| | | | Mass spectrum |
|---|---|---|---|
| Example | Structure | Name | (apci) |
| 23 | 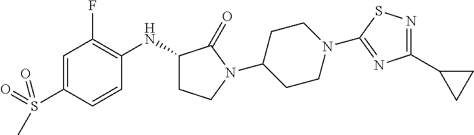 [SMILES: CS(=O)(=O)c1ccc(N[C@H]2CCN(C3CCN(c4nc(C5CC5)ns4)CC3)C2=O)c(F)c1] | (S)-1-(1-(3-cyclopropyl-1,2,4- thiadiazol-5-yl)piperidin-4-yl)- 3-(2-fluoro-4-(methyl- sulfonyl)phenylamino) pyrrolidin-2-one | m/z = 480.2 (M + H) |
| 24 | 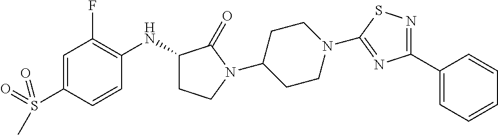 [SMILES: CS(=O)(=O)c1ccc(N[C@H]2CCN(C3CCN(c4nc(-c5ccccc5)ns4)CC3)C2=O)c(F)c1] | (S)-3-(2-fluoro-4- (methylsulfonyl)phenylamino)- 1-(1-(3-phenyl-1,2,4- thiadiazol-5-yl)piperidin-4- yl)pyrrolidin-2-one | m/z = 516.2 (M + H) |

### Example 25

### (S)-1-(1-(3-((S)-1,2-dihydroxyethyl)-1,2,4-thiadiazol-5-yl)piperidin-4-yl)-3-(2-fluoro-4-(methylsulfonyl)phenylamino)pyrrolidin-2-one

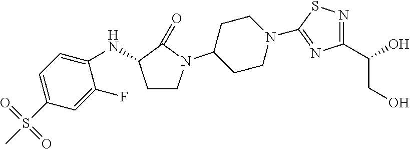 [SMILES: CS(=O)(=O)c1ccc(N[C@H]2CCN(C3CCN(c4nc([C@H](O)CO)ns4)CC3)C2=O)c(F)c1]

(S)-1-(1-(3-((S)-1,4-dioxaspiro[4.5]decan-2-yl)-1,2,4-thiadiazol-5-yl)piperidin-4-yl)-3-(2-fluoro-4-(methylsulfonyl)phenylamino)pyrrolidin-2-one (prepared according to the method of Example 22; 0.400 g, 0.690 mmol) was dissolved in ethanol (25 mL). Water (400 mg) and concentrated HCl (400 mg) were added. The mixture was heated to reflux for 2 hours and then concentrated. Ethanol (2×50 mL) was added and then removed in vacuo. The residue was triturated from MeCN to give (S)-1-(1-(3-((S)-1,2-dihydroxyethyl)-1,2,4-thiadiazol-5-yl)piperidin-4-yl)-3-(2-fluoro-4-(methylsulfonyl)phenylamino)pyrrolidin-2-one (0.30 g, 0.60 mmol, 87%). Mass Spectrum (apci) m/z=500 (M+H).

### Example 26

### (S)-3-((2,5-Difluoro-4-(methylsulfonyl)phenyl)(methyl)amino)-1-(1-(3-isopropyl-1,2,4-thiadiazol-5-yl)piperidin-4-yl)pyrrolidin-2-one

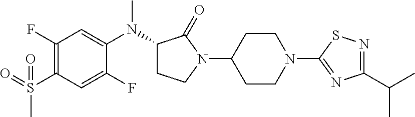 [SMILES: CC(C)c1nsc(N2CCC(N3CC[C@H](N(C)c4cc(F)c(S(C)(=O)=O)cc4F)C3=O)CC2)n1]

Step A:

(S)-tert-Butyl 1-(1-(3-isopropyl-1,2,4-thiadiazol-5-yl)piperidin-4-yl)-2-oxopyrrolidin-3-ylcarbamate (Example 1, Steps A-C; 300 mg, 0.733 mmol) was dissolved in DMF (7 mL) and cooled in an ice bath. 60% Sodium hydride (32.2 mg, 0.806 mmol) was added and the reaction was stirred for 5 minutes. Iodomethane (68.7 μL, 1.10 mmol) was added and the reaction was allowed to warm to ambient temperature. After 4 hours, additional NaH (15 mg) and MeI (50 μL) were added. After 1 hour, the reaction was poured into water. A white precipitate crashed out and was filtered and dried to afford (5)-tert-butyl 1-(1-(3-isopropyl-1,2,4-thiadiazol-5-yl)piperidin-4-yl)-2-oxopyrrolidin-3-yl(methyl)carbamate (284 mg, 0.670 mmol, 91.5% yield).

Step B:

(S)-tert-Butyl 1-(1-(3-isopropyl-1,2,4-thiadiazol-5-yl)piperidin-4-yl)-2-oxopyrrolidin-3-yl(methyl)carbamate (284 mg, 0.670 mmol) was dissolved in CH₂Cl₂ (5 mL), TFA (2 mL) was added and the reaction was stirred at ambient temperature for 30 minutes. The reaction was concentrated, partitioned between saturated aqueous NaHCO₃ and CH₂Cl₂ and extracted with CH₂Cl₂. The combined organic layers were dried over Na₂SO₄, filtered and concentrated to afford (S)-1-(1-(3-isopropyl-1,2,4-thiadiazol-5-yl)piperidin-4-yl)-3-(methylamino)pyrrolidin-2-one (228 mg, 0.705 mmol, 105% yield).

Step C:

(S)-1-(1-(3-isopropyl-1,2,4-thiadiazol-5-yl)piperidin-4-yl)-3-(methylamino)pyrrolidin-2-one (110 mg, 0.340 mmol), 1,2,4-trifluoro-5-(methylsulfonyl)benzene (107 mg, 0.510 mmol) and Na₂CO₃ (72.1 mg, 0.680 mmol) were dissolved in DMSO (5 mL) and the mixture was heated to 120° C. under nitrogen for 24 hours. The reaction was cooled to ambient temperature, partitioned between water and CH₂Cl₂ and extracted with CH₂Cl₂. The combined organic layers were dried over Na₂SO₄, filtered and concentrated. The residue was purified over silica gel (90% EtOAc in hexanes) to afford (S)-3-((2,5-difluoro-4-(methylsulfonyl)phenyl)(methyl)amino)-1-(1-(3-isopropyl-1,2,4-thiadiazol-5-yl)piperidin-4-yl)pyrrolidin-2-one (48.9 mg, 0.0952 mmol, 28.0% yield) as a white solid. Mass spectrum (apci) m/z=514.2 (M+H).

### Example 27

### (S)-3-(2-fluoro-4-(methylsulfonyl)phenylamino)-1′-(3-isopropyl-1,2,4-thiadiazol-5-yl)-[1,4′-bipiperidin]-2-one

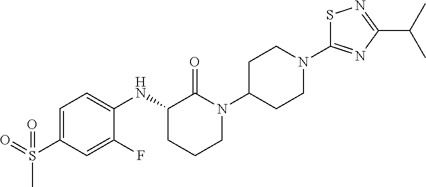 [SMILES: CC(C)c1nsc(N2CCC(N3CCC[C@H](Nc4ccc(S(C)(=O)=O)cc4F)C3=O)CC2)n1]

Step A:

(S)-tert-butyl 3-(2-fluoro-4-(methylsulfonyl)phenylamino)-2-oxo-1,4′-bipiperidine-1′-carboxylate (Preparation LL; 570 mg, 1.21 mmol) was dissolved in CH₂Cl₂ (10 mL) and trifluoroacetic acid (2.8 g, 24.3 mmol) was added. The reaction was stirred at ambient temperature for 30 minutes. The reaction was concentrated in vacuo, partitioned between saturated aqueous NaHCO₃ and CH₂Cl₂, and extracted with 10% methanol in CH₂Cl₂. The combined organic layers were dried over Na₂SO₄, filtered and concentrated to afford (S)-3-(2-fluoro-4-(methylsulfonyl)phenylamino)-[1,4′-bipiperidin]-2-one (260 mg, 0.704 mmol, 58.0% yield).

Step B:

N-(Methylsulfonyloxy)isobutyrimidoyl chloride (141 mg, 0.704 mmol) was dissolved in EtOAc (7 mL). Sodium thiocyanate (57.1 mg, 0.704 mmol) and pyridine (227 μL, 2.81 mmol) were added and the reaction was heated to 45° C. for 45 minutes. (S)-3-(2-Fluoro-4-(methylsulfonyl)phenylamino)-[1,4′-bipiperidin]-2-one (260 mg, 0.704 mmol) was added and the reaction was heated to 70° C. over the weekend. The reaction was cooled to ambient temperature, partitioned between water and EtOAc, dried over Na₂SO₄, filtered and concentrated. The residue was purified over silica gel (100% EtOAc) to afford (S)-3-(2-fluoro-4-(methylsulfonyl)phenylamino)-1′-(3-isopropyl-1,2,4-thiadiazol-5-yl)-[1,4′-bipiperidin]-2-one (125 mg, 0.252 mmol, 35.8% yield) as a white solid. Mass spectrum (apci) m/z=496.3 (M+H).

The following compounds were also prepared according to the method of Example 27.

| | | | Mass spectrum |
|---|---|---|---|
| Example | Structure | Name | (apci) |
| 28 | 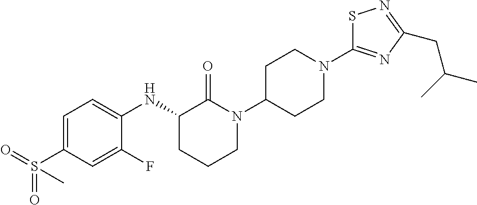 [SMILES: CC(C)Cc1nsc(N2CCC(N3CCC[C@H](Nc4ccc(S(C)(=O)=O)cc4F)C3=O)CC2)n1] | (S)-3-(2-fluoro-4- (methylsulfonyl)phenylamino)- 1′-(3-isobutyl-1,2,4-thiadiazol- 5-yl)-[1,4′-bipiperidin]-2-one | m/z = 510.3 (M + H). |
| 29 | 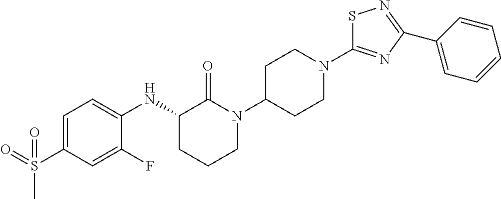 [SMILES: CS(=O)(=O)c1ccc(N[C@H]2CCCN(C3CCN(c4nc(-c5ccccc5)ns4)CC3)C2=O)c(F)c1] | (S)-3-(2-fluoro-4- (methylsulfonyl)phenylamino)- 1′-(3-phenyl-1,2,4-thiadiazol- 5-yl)-[1,4′-bipiperidin]-2-one | m/z = 530.2 (M + H). |

### Example 30

### (S)-1′-(3-tert-butyl-1,2,4-thiadiazol-5-yl)-3-(2-fluoro-4-(methylsulfonyl)phenylamino)-[1,4′-bipiperidin]-2-one

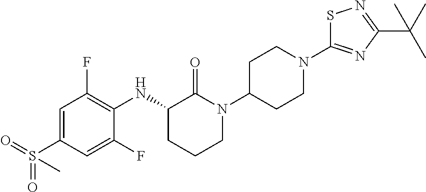 [SMILES: CC(C)(C)c1nsc(N2CCC(N3CCC[C@H](Nc4c(F)cc(S(C)(=O)=O)cc4F)C3=O)CC2)n1]

A flask was charged with (S)-3-(2-fluoro-4-(methylsulfonyl)phenylamino)-[1,4′-bipiperidin]-2-one hydrochloride (Example 27, Step A; 0.200 g, 0.493 mmol), 3-tert-butyl-5-chloro-1,2,4-thiadiazole (0.0870 g, 0.493 mmol), TEA (0.275 mL, 1.97 mmol), and ethanol (5 mL). The mixture was stirred at 72° C. overnight. The mixture was concentrated in vacuo and the product was purified using silica gel column chromatography, eluting with 25-50-75% ethyl acetate in hexanes to provide (S)-1′-(3-tert-butyl-1,2,4-thiadiazol-5-yl)-3-(2-fluoro-4-(methylsulfonyl)phenylamino)-[1,4′-bipiperidin]-2-one (0.046 g, 0.0875 mmol, 17.8% yield) as light yellow solid. Mass spectrum (apci) m/z=510.3 (M+H).

### Example 31

### (S)-1′-(3-tert-butyl-1,2,4-thiadiazol-5-yl)-3-(2,6-difluoro-4-ethylsulfonyl)phenylamino)-[1,4′-bipiperidin]-2-one

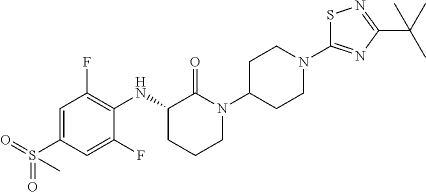 [SMILES: CC(C)(C)c1nsc(N2CCC(N3CCC[C@H](Nc4c(F)cc(S(C)(=O)=O)cc4F)C3=O)CC2)n1]

Step A:

(S)-tert-Butyl 3-(2,6-difluoro-4-(methylsulfonyl)phenylamino)-2-oxo-1,4′-bipiperidine-1′-carboxylate (Preparation MM; 6.00 g, 12.3 mmol) was dissolved in DCM (20 mL). TFA (10 mL) was added, and the reaction was stirred for 1 hour. The reaction mixture was concentrated, neutralized with saturated sodium bicarbonate, and extracted with a mixture of CHCl₃/iPA (3×50 mL). The combined organic layers were dried over MgSO₄, filtered and concentrated to give (S)-3-(2,6-difluoro-4-(methylsulfonyl)phenylamino)-[1,4′-bipiperidin]-2-one (3.88 g, 10.0 mmol, 81.4% yield).

Step B:

A flask was charged with (S)-3-(2,6-difluoro-4-(methylsulfonyl)phenylamino)-[1,4′-bipiperidin]-2-one hydrochloride (0.2 g, 0.472 mmol), 3-tert-butyl-5-chloro-1,2,4-thiadiazole (0.0834 g, 0.472 mmol), triethylamine (0.263 mL, 1.89 mmol), and ethanol (5 mL). The mixture was stirred at 72° C. overnight. The solvent was removed in vacuo and the crude product was purified by silica gel column chromatography, eluting with 25-50-75% ethyl acetate in hexanes, to provide (S)-1′-(3-tert-butyl-1,2,4-thiadiazol-5-yl)-3-(2,6-difluoro-4-(methylsulfonyl)phenylamino)-[1,4′-bipiperidin]-2-one (0.164 g, 0.308 mmol, 65.2% yield) as light yellow solid. Mass spectrum (apci) m/z=528.3 (M+H).

### Example 32

### 1-(1-(3-Isopropyl-1,2,4-thiadiazol-5-yl)piperidin-4-yl)-3-(4-(methylthio)phenoxy)pyrrolidin-2-one

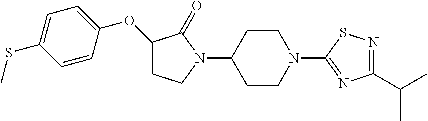 [SMILES: CSc1ccc(OC2CCN(C3CCN(c4nc(C(C)C)ns4)CC3)C2=O)cc1]

Step A:

1-(3-Isopropyl-1,2,4-thiadiazol-5-yl)piperidin-4-amine hydrochloride (500 mg, 1.90 mmol) was suspended in THF (15 mL). Triethylamine (796 μL, 5.71 mmol) was added followed by 2,4-dibromobutanoyl chloride (251 μL, 1.90 mmol). The reaction was stirred at ambient temperature for 1 hour, partitioned between CH₂Cl₂ and water, dried over Na₂SO₄, filtered and concentrated to afford 2,4-dibromo-N-(1-(3-isopropyl-1,2,4-thiadiazol-5-yl)piperidin-4-yl)butanamide (743 mg, 1.64 mmol, 86.0% yield), which was used directly in the next step without purification.

Step B:

2,4-Dibromo-N-(1-(3-isopropyl-1,2,4-thiadiazol-5-yl)piperidin-4-yl)butanamide (743 mg, 1.64 mmol) was dissolved in DMF (10 mL) and 60% sodium hydride (65.4 mg, 1.64 mmol) was added. The reaction was stirred at ambient temperature overnight. The reaction was poured into water, extracted with EtOAc, washed with brine, dried over Na₂SO₄, filtered and concentrated. The residue was purified over silica gel (75% EtOAc in hexanes) to afford 3-bromo-1-(1-(3-isopropyl-1,2,4-thiadiazol-5-yl)piperidin-4-yl)pyrrolidin-2-one (380 mg, 1.02 mmol, 62.2% yield).

Step C:

3-Bromo-1-(1-(3-isopropyl-1,2,4-thiadiazol-5-yl)piperidin-4-yl)pyrrolidin-2-one (380 mg, 1.02 mmol) was dissolved in DMF (10 mL). 4-(Methylthio)phenol (214 mg, 1.53 mmol) and K₂CO₃ (141 mg, 1.02 mmol) were added and the reaction was heated to 100° C. for 30 minutes. The reaction was cooled to ambient temperature, partitioned between water and EtOAc, dried over Na₂SO₄, filtered and concentrated. The residue was purified over silica gel (60% EtOAc in hexanes) to afford 1-(1-(3-isopropyl-1,2,4-thiadiazol-5-yl)piperidin-4-yl)-3-(4-(methylthio)phenoxy)pyrrolidin-2-one (138 mg, 0.319 mmol, 31.3% yield) as a white solid. Mass spectrum (apci) m/z=433.2 (M+H).

The following compound was also prepared according to the method of Example 32.

| | | | Mass spectrum |
|---|---|---|---|
| Example | Structure | Name | (apci) |
| 33 | 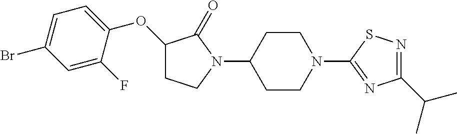 [SMILES: CC(C)c1nsc(N2CCC(N3CCC(Oc4ccc(Br)cc4F)C3=O)CC2)n1] | 3-(4-bromo-2-fluorophenoxy)- 1-(1-(3-isopropyl-1,2,4- thiadiazol-5-yl)piperidin-4- yl)pyrrolidin-2-one | m/z = 483.1, 485.1 (M + H). |

### Example 34

### (S)-1-(1-(3-tert-butyl-1,2,4-thiadiazol-5-yl)piperidin-4-yl)-3-(2,5-difluoro-4-(methylsulfonyl)phenoxy)pyrrolidin-2-one

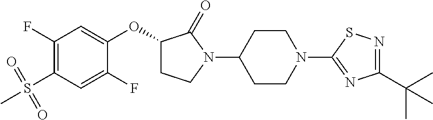 [SMILES: CC(C)(C)c1nsc(N2CCC(N3CC[C@H](Oc4cc(F)c(S(C)(=O)=O)cc4F)C3=O)CC2)n1]

(S)-3-(2,5-Difluoro-4-(methylsulfonyl)phenoxy)-1-(piperidin-4-yl)pyrrolidin-2-one (Preparation JJ; 200 mg, 0.534 mmol) was dissolved in EtOH (3 mL) and 3-tert-butyl-5-chloro-1,2,4-thiadiazole (94.4 mg, 0.534 mmol) and triethylamine (298 μL, 2.14 mmol) were added. The reaction was heated to reflux overnight. The reaction was cooled and partitioned between water and EtOAc, dried over Na₂SO₄, filtered and concentrated. The residue was purified over silica gel (80% EtOAc in hexanes) to afford (S)-1-(1-(3-tert-butyl-1,2,4-thiadiazol-5-yl)piperidin-4-yl)-3-(2,5-difluoro-4-(methylsulfonyl)phenoxy)pyrrolidin-2-one (220 mg, 0.428 mmol, 80.0% yield) as a white solid. Mass spectrum (apci) m/z=515.3 (M+H).

The following compounds were also prepared according to the method of Example 34.

| | | | Mass spectrum |
|---|---|---|---|
| Example | Structure | Name | (apci) |
| 35 | 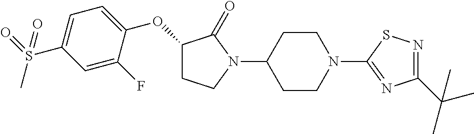 [SMILES: CC(C)(C)c1nsc(N2CCC(N3CC[C@H](Oc4ccc(S(C)(=O)=O)cc4F)C3=O)CC2)n1] | (S)-1-(1-(3-tert-butyl- 1,2,4-thiadiazol-5- yl)piperidin-4-yl)-3-(2- fluoro-4-(methylsulfonyl) phenoxy)pyrrolidin-2-one | m/z = 497.2 (M + H). |
| 36 | 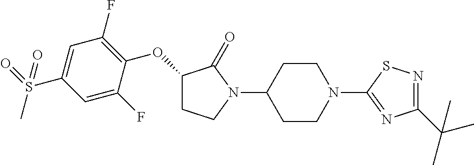 [SMILES: CC(C)(C)c1nsc(N2CCC(N3CC[C@H](Oc4c(F)cc(S(C)(=O)=O)cc4F)C3=O)CC2)n1] | (S)-1-(1-(3-tert-butyl- 1,2,4-thiadiazol-5- yl)piperidin-4-yl)-3-(2,6- difluoro-4- (methylsulfonyl)phenoxy) pyrrolidin-2-one | m/z = 515.3 (M + H). |
| 37 | 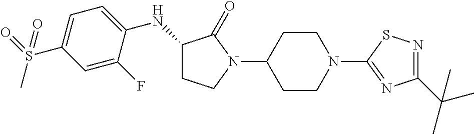 [SMILES: CC(C)(C)c1nsc(N2CCC(N3CC[C@H](Nc4ccc(S(C)(=O)=O)cc4F)C3=O)CC2)n1] | (S)-1-(1-(3-tert-butyl- 1,2,4-thiadiazol-5- yl)piperidin-4-yl)-3-(2- fluoro-4- (methylsulfonyl)phenyl- amino)pyrrolidin-2-one | m/z = 496.3 (M + H). |
| 38 | 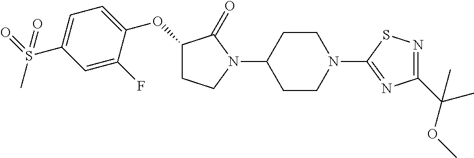 [SMILES: COC(C)(C)c1nsc(N2CCC(N3CC[C@H](Oc4ccc(S(C)(=O)=O)cc4F)C3=O)CC2)n1] | (S)-3-(2-fluoro-4- (methylsulfonyl)phenoxy)- 1-(1-(3-(2-methoxypropan- 2-yl)-1,2,4-thiadiazol-5- yl)piperidin-4- yl)pyrrolidin-2-one | Mass spectrum (apci) m/z = 512.9 (M + H). |
| 39 | 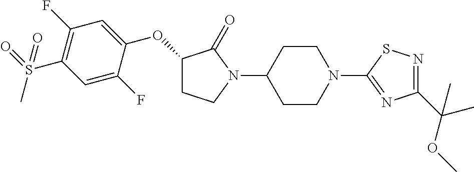 [SMILES: COC(C)(C)c1nsc(N2CCC(N3CC[C@H](Oc4cc(F)c(S(C)(=O)=O)cc4F)C3=O)CC2)n1] | (S)-3-(2,5-difluoro-4- (methylsulfonyl)phenoxy)- 1-(1-(3-(2-methoxypropan- 2-yl)-1,2,4-thiadiazol-5- yl)piperidin-4- yl)pyrrolidin-2-one | m/z = 531.0 (M + H). |
| 40 | 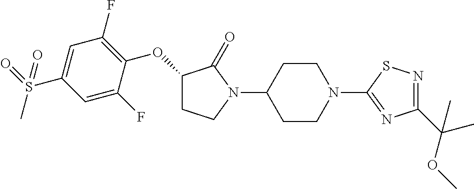 [SMILES: COC(C)(C)c1nsc(N2CCC(N3CC[C@H](Oc4c(F)cc(S(C)(=O)=O)cc4F)C3=O)CC2)n1] | (S)-3-(2,6-difluoro-4- (methylsulfonyl)phenoxy)- 1-(1-(3-(2-methoxypropan- 2-yl)-1,2,4-thiadiazol-5- yl)piperidin-4- yl)pyrrolidin-2-one | m/z = 530.9 (M + H). |
| 41 | 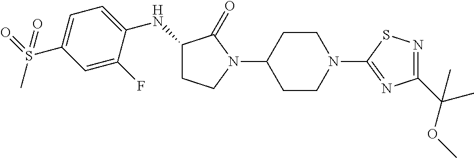 [SMILES: COC(C)(C)c1nsc(N2CCC(N3CC[C@H](Nc4ccc(S(C)(=O)=O)cc4F)C3=O)CC2)n1] | (S)-3-(2-fluoro-4- (methylsulfonyl)phenyl- amino)-1-(1-(3-(2- methoxypropan-2-yl)- 1,2,4-thiadiazol-5- yl)piperidin-4- yl)pyrrolidin-2-one | m/z = 512.0 (M + H). |
| 42 | 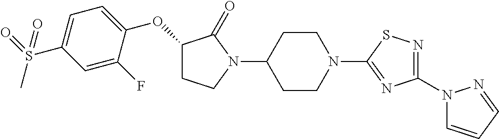 [SMILES: CS(=O)(=O)c1ccc(O[C@H]2CCN(C3CCN(c4nc(-n5cccn5)ns4)CC3)C2=O)c(F)c1] | (S)-1-(1-(3-(1H-pyrazol-1- yl)-1,2,4-thiadiazol-5- yl)piperidin-4-yl)-3-(2- fluoro-4-(methylsulfonyl) phenoxy)pyrrolidin-2-one | m/z = 507.2 (M + H). |

### Example 43

### (S)-3-(2-fluoro-4-(5-methyl-1,3,4-oxadiazol-2-yl)phenoxy)-1-(1-(3-isopropyl-1,2,4-thiadiazol-5-yl)piperidin-4-yl)pyrrolidin-2-one

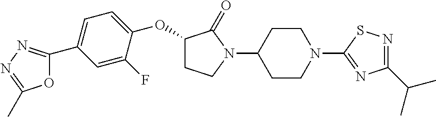 [SMILES: Cc1nnc(-c2ccc(O[C@H]3CCN(C4CCN(c5nc(C(C)C)ns5)CC4)C3=O)c(F)c2)o1]

Step A:

(R)-tert-butyl 4-(3-(methylsulfonyloxy)-2-oxopyrrolidin-1-yl)piperidine-1-carboxylate (Preparation EE; 1.0 g, 2.8 mmol) was dissolved in dry DMSO (15 mL). Methyl 3-fluoro-4-hydroxybenzoate (0.56 g, 3.3 mmol) and K₂CO₃ (0.46 g, 3.3 mmol) were added and the reaction was heated to 70° C. under nitrogen. After 3 hours the reaction was cooled to ambient temperature and stirred at ambient temperature overnight. The reaction was partitioned between water and EtOAc, extracted with EtOAc, and washed with brine. The combined organic layers were dried over Na₂SO₄, filtered and concentrated. The residue was purified over silica gel (60% EtOAc in hexanes) to afford (S)-tert-butyl 4-(3-(2-fluoro-4-(methoxycarbonyl)phenoxy)-2-oxopyrrolidin-1-yl)piperidine-1-carboxylate (1.2 g, 2.7 mmol, 100% yield) as a white solid.

Step B:

(S)-tert-butyl 4-(3-(2-fluoro-4-(methoxycarbonyl)phenoxy)-2-oxopyrrolidin-1-yl)piperidine-1-carboxylate (900 mg, 2.06 mmol) was dissolved in THF (20 mL). Potassium trimethylsilanolate (529 mg, 4.12 mmol) was added and the reaction was stirred at ambient temperature for 2 hours. Potassium trimethylsilanolate (529 mg, 4.12 mmol) was added and the mixture was stirred for 3 hours. The reaction was poured into 1N HCl and extracted with EtOAc, dried over Na₂SO₄, filtered and concentrated to afford (S)-4-(1-(1-(tert-butoxycarbonyl)piperidin-4-yl)-2-oxopyrrolidin-3-yloxy)-3-fluorobenzoic acid (834 mg, 1.97 mmol, 95.7% yield) as a white solid.

Step C:

(S)-4-(1-(1-(tert-Butoxycarbonyl)piperidin-4-yl)-2-oxopyrrolidin-3-yloxy)-3-fluorobenzoic acid (834 mg, 1.97 mmol), acetohydrazide (219 mg, 2.96 mmol) and N-ethyl-N-isopropylpropan-2-amine (1375 μL, 7.90 mmol) were dissolved in CH₂Cl₂ (20 mL). Bis(2-oxooxazolidin-3-yl)phosphinic chloride (2010 mg, 7.90 mmol) was added and the mixture was stirred at ambient temperature for 3 hours. The reaction was poured into water, extracted with EtOAc, washed with brine, dried over Na₂SO₄, filtered and concentrated to afford crude (5)-tert-butyl 4-(3-(4-(2-acetylhydrazinecarbonyl)-2-fluorophenoxy)-2-oxopyrrolidin-1-yl)piperidine-1-carboxylate (960 mg, 2.01 mmol, 102% yield), which was taken forward without further purification.

Step D:

(S)-tert-Butyl 4-(3-(4-(2-acetylhydrazinecarbonyl)-2-fluorophenoxy)-2-oxopyrrolidin-1-yl)piperidine-1-carboxylate (960 mg, 2.01 mmol) was dissolved in CH₂Cl₂ (20 mL) and the mixture was cooled to 0° C. under nitrogen. 1H-imidazole (341 mg, 5.02 mmol), triphenylphosphine (1158 mg, 4.41 mmol) and perbromomethane (1464 mg, 4.41 mmol) were added and the reaction was allowed to warm to ambient temperature overnight. The reaction was partitioned between water and EtOAc, dried over Na₂SO₄, filtered and concentrated. The residue was purified over silica gel (100% EtOAc) to afford (S)-tert-butyl 4-(3-(2-fluoro-4-(5-methyl-1,3,4-oxadiazol-2-yl)phenoxy)-2-oxopyrrolidin-1-yl)piperidine-1-carboxylate (680 mg, 1.48 mmol, 73.6% yield).

Step E:

(S)-tert-Butyl 4-(3-(2-fluoro-4-(5-methyl-1,3,4-oxadiazol-2-yl)phenoxy)-2-oxopyrrolidin-1-yl)piperidine-1-carboxylate (680 mg, 1.48 mmol) was dissolved in CH₂Cl₂ (10 mL) and TFA (4 mL) was added. The mixture was stirred at ambient temperature for 1 hour and then concentrated in vacuo to afford crude (S)-3-(2-fluoro-4-(5-methyl-1,3,4-oxadiazol-2-yl)phenoxy)-1-(piperidin-4-yl)pyrrolidin-2-one 2,2,2-trifluoroacetate as an oil which was taken forward without further purification.

Step F:

(S)-3-(2-Fluoro-4-(5-methyl-1,3,4-oxadiazol-2-yl)phenoxy)-1-(piperidin-4-yl)pyrrolidin-2-one 2,2,2-trifluoroacetate (150 mg, 0.316 mmol) was dissolved in DMF (3 mL). N-ethyl-N-isopropylpropan-2-amine (138 μL, 0.790 mmol) and 5-chloro-3-isopropyl-1,2,4-thiadiazole (61.7 mg, 0.379 mmol) were added and the reaction was heated to 100° C. for 2 hours. The reaction was cooled to ambient temperature, water was added and the mixture was extracted with EtOAc, washed with brine, dried over Na₂SO₄, filtered and concentrated. The residue was purified twice over silica gel (100% EtOAc) to afford (S)-3-(2-fluoro-4-(5-methyl-1,3,4-oxadiazol-2-yl)phenoxy)-1-(1-(3-isopropyl-1,2,4-thiadiazol-5-yl)piperidin-4-yl)pyrrolidin-2-one (21.6 mg, 0.0444 mmol, 14.0% yield). Mass spectrum (apci) m/z=487.2 (M+H).

### Example 44

### 3-(4-(Cyclopropylmethylsulfonyl)-2-fluorophenoxy)-1-(1-(3-isopropyl-1,2,4-thiadiazol-5-yl)piperidin-4-yl)pyrrolidin-2-one trifluoroacetate

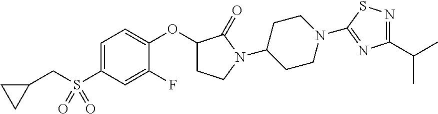 [SMILES: CC(C)c1nsc(N2CCC(N3CCC(Oc4ccc(S(=O)(=O)CC5CC5)cc4F)C3=O)CC2)n1]

Step A:

To a solution of 3-(4-bromo-2-fluorophenoxy)-1-(1-(3-isopropyl-1,2,4-thiadiazol-5-yl)piperidin-4-yl)pyrrolidin-2-one (Example 33; 0.50 g, 1.0 mmol) in dioxane (10 mL) continuously purged with nitrogen was added (9,9-dimethyl-9H-xanthene-4,5-diyl)bis(diphenylphosphine) (0.06 g, 0.10 mmol), N-ethyl-N-isopropylpropan-2-amine (0.23 mL, 1.2 mmol), Pd₂ dba₃ (0.05 g, 0.05 mmol), and methyl 3-mercaptopropanoate (0.84 g, 6.99 mmol). The reaction was heated to 80° C. for 4 hours. The reaction was concentrated in vacuo and the residue was purified over silica gel (25% EtOAc/CH₂Cl₂) to yield methyl 3-(3-fluoro-4-(1-(1-(3-isopropyl-1,2,4-thiadiazol-5-yl)piperidin-4-yl)-2-oxopyrrolidin-3-yloxy)phenylthio)propanoate (0.50 g, 92%).

Step B:

To a solution of methyl 3-(3-fluoro-4-(1-(1-(3-isopropyl-1,2,4-thiadiazol-5-yl)piperidin-4-yl)-2-oxopyrrolidin-3-yloxy)phenylthio)propanoate (0.50 g, 0.96 mmol) in THF (10 mL) was added 1M potassium 2-methylpropan-2-olate in THF (1.9 mL, 1.9 mmol). The reaction was stirred for 5 minutes at ambient temperature, followed by addition of (bromomethyl)cyclopropane (0.26 g, 1.9 mmol) and reaction was stirred for 1 hour at ambient temperature. The reaction was poured into water, extracted with EtOAc, washed with brine, dried over MgSO₄ and concentrated in vacuo. The residue was purified over silica gel (30% EtOAc/CH₂Cl₂) to yield 3-(4-(cyclopropylmethylthio)-2-fluorophenoxy)-1-(1-(3-isopropyl-1,2,4-thiadiazol-5-yl)piperidin-4-yl)pyrrolidin-2-one (0.16 g, 34%).

Step C:

To a solution of 3-(4-(cyclopropylmethylthio)-2-fluorophenoxy)-1-(1-(3-isopropyl-1,2,4-thiadiazol-5-yl)piperidin-4-yl)pyrrolidin-2-one (0.16 g, 0.33 mmol) in CH₂Cl₂ (10 mL) was added 3-chlorobenzoperoxoic acid (0.24 g, 0.98 mmol) and the reaction was stirred at ambient temperature for 2 hours. The reaction was concentrated in vacuo and the solids were partitioned between EtOAc and 1N sodium hydroxide. The combined organic layers were washed with 1N sodium hydroxide, and brine, dried over MgSO₄, filtered and concentrated in vacuo. The residue was purified over silica gel (100% EtOAc) to yield a foam. The foam was purified by reverse phase chromatography (5 to 95% acetonitrile in water with 0.1% TFA) to yield 3-(4-(cyclopropylmethylsulfonyl)-2-fluorophenoxy)-1-(1-(3-isopropyl-1,2,4-thiadiazol-5-yl)piperidin-4-yl)pyrrolidin-2-one (0.0076 g, 0.015 mmol, 4.5% yield). Mass Spectrum (apci) m/z=523 (M+H).

The following compound was prepared according to the procedure of Example 44.

| | | | Mass spectrum |
|---|---|---|---|
| Example | Structure | Name | (apci) |
| 45 | 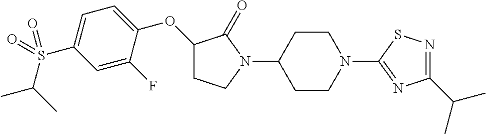 [SMILES: CC(C)c1nsc(N2CCC(N3CCC(Oc4ccc(S(=O)(=O)C(C)C)cc4F)C3=O)CC2)n1] | 3-(2-fluoro-4- (isopropylsulfonyl)phenoxy)- 1-(1-(3-isopropyl-1,2,4- thiadiazol-5-yl)piperidin-4- yl)pyrrolidin-2-one | m/z = 511.2 (M + H) |

### Example 46

### 3-(4-(Cyclopropylsulfonyl)-2-fluorophenoxy)-1-(1-(3-isopropyl-1,2,4-thiadiazol-5-yl)piperidin-4-yl)pyrrolidin-2-one trifluoroacetate

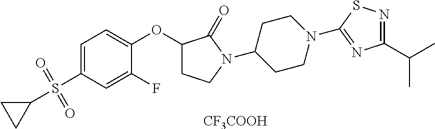 [SMILES: CC(C)c1nsc(N2CCC(N3CCC(Oc4ccc(S(=O)(=O)C5CC5)cc4F)C3=O)CC2)n1 ; "CF3COOH"]

Step A:

To a solution of methyl 3-(3-fluoro-4-(1-(1-(3-isopropyl-1,2,4-thiadiazol-5-yl)piperidin-4-yl)-2-oxopyrrolidin-3-yloxy)phenylthio)propanoate (Example 44, Step A; 1.4 g, 2.7 mmol) in THF (50 mL) was added potassium 2-methylpropan-2-olate (8.0 mL, 8.0 mmol) and the reaction was stirred for 5 minutes at ambient temperature, followed by the addition of water (10 mL) and 1-chloro-3-iodopropane (0.86 mL, 8.0 mmol). The reaction was stirred at ambient temperature for 2 hours. The reaction was poured into water and extracted into EtOAc. The combined organic layers were washed with brine, dried over MgSO₄, filtered and concentrated in vacuo. The crude material was purified over silica gel (30% EtOAc/CH₂Cl₂) to yield 3-(4-(3-chloropropylthio)-2-fluorophenoxy)-1-(1-(3-isopropyl-1,2,4-thiadiazol-5-yl)piperidin-4-yl)pyrrolidin-2-one (1.2 g, 86%).

Step B:

To a solution of 3-(4-(3-chloropropylthio)-2-fluorophenoxy)-1-(1-(3-isopropyl-1,2,4-thiadiazol-5-yl)piperidin-4-yl)pyrrolidin-2-one (1.2 g, 2.3 mmol) in CH₂Cl₂ (40 mL) cooled to 0° C. was added 3-chlorobenzoperoxoic acid (1.3 g, 5.4 mmol) and the reaction was stirred for 2 hours. The reaction was poured into water and extracted into EtOAc. The combined organic layers were washed with 1N sodium hydroxide and brine, dried over MgSO₄, filtered and concentrated in vacuo. The residue was purified over silica gel (100% EtOAc) to yield 3-(4-(3-chloropropylsulfonyl)-2-fluorophenoxy)-1-(1-(3-isopropyl-1,2,4-thiadiazol-5-yl)piperidin-4-yl)pyrrolidin-2-one (0.10 g, 7.8%).

Step C:

A solution of 3-(4-(3-chloropropylsulfonyl)-2-fluorophenoxy)-1-(1-(3-isopropyl-1,2,4-thiadiazol-5-yl)piperidin-4-yl)pyrrolidin-2-one (0.10 g, 0.183 mmol) in THF (10 mL) was cooled to −78° C. Sodium hexamethyl disilylazide (0.55 mL, 0.550 mmol, 1 M solution in THF) was added and the reaction was stirred at −78° C. for 1 hour. Water was added at −78° C. The mixture was added to water and extracted into EtOAc. The combined organic layers were washed with brine, dried over MgSO₄, filtered and concentrated in vacuo. The residue was purified by reverse phase HPLC (5 to 95% acetonitrile in water with 0.1% TFA) to yield 3-(4-(cyclopropylsulfonyl)-2-fluorophenoxy)-1-(1-(3-isopropyl-1,2,4-thiadiazol-5-yl)piperidin-4-yl)pyrrolidin-2-one (0.0247 g, 0.0486 mmol, 26.5% yield). Mass Spectrum (apci) m/z=509.2 (M+H).

### Example 47

### 1-(1-(3-Cyclopropyl-1,2,4-thiadiazol-5-yl)piperidin-4-yl)-3-(2,5-difluoro-4-(methylsulfonyl)phenoxy)pyrrolidin-2-one trifluoroacetate

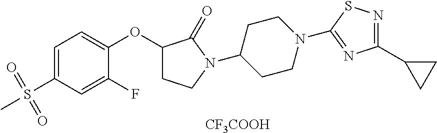 [SMILES: CS(=O)(=O)c1ccc(OC2CCN(C3CCN(c4nc(C5CC5)ns4)CC3)C2=O)c(F)c1 ; "CF3COOH"]

Step A:

Tert-butyl 4-aminopiperidine-1-carboxylate (10 g, 49.9 mmol) and triethylamine (7.66 mL, 54.9 mmol) were dissolved in THF (250 mL). 2,4-Dibromobutanoyl chloride (6.60 mL, 49.9 mmol) was added slowly and the reaction was stirred at ambient temperature for 30 minutes. The resultant solids were filtered and the filtrate was concentrated in vacuo to afford crude tert-butyl 4-(2,4-dibromobutanamido)piperidine-1-carboxylate (21.4 g, 50.0 mmol, 100% yield) which was taken on to the next reaction without further purification.

Step B:

Tert-butyl 4-(2,4-dibromobutanamido)piperidine-1-carboxylate (21.4 g, 50.0 mmol) was dissolved in DMF (250 mL) and 60% sodium hydride (2.00 g, 50.0 mmol) was added and the reaction was stirred at ambient temperature for 2 hours. The reaction was concentrated in vacuo, partitioned between aqueous NH₄Cl and EtOAc, dried over Na₂SO₄, filtered and concentrated. The residue was purified over silica gel to afford tert-butyl 4-(3-bromo-2-oxopyrrolidin-1-yl)piperidine-1-carboxylate (12.5 g, 36.0 mmol, 72.0% yield).

Step C:

To a solution of potassium carbonate (4.78 g, 34.6 mmol) in acetone was added 4-bromo-2,5-difluorophenol (4.87 g, 23.3 mmol) and the reaction was stirred for 10 minutes, followed by the addition of tert-butyl 4-(3-bromo-2-oxopyrrolidin-1-yl)piperidine-1-carboxylate (6.0 g, 17.3 mmol). The reaction was stirred overnight at ambient temperature. The reaction was filtered and concentrated in vacuo and the residue partitioned between EtOAc and 1N NaOH. The combined organic layers were washed with water, brine, dried over MgSO₄, filtered and concentrated in vacuo. The residue was purified over silica gel (20% EtOAc/CH₂Cl₂) to yield tert-butyl 4-(3-(4-bromo-2,5-difluorophenoxy)-2-oxopyrrolidin-1-yl)piperidine-1-carboxylate (5.7 g, 69% yield).

Step D:

A solution of tert-butyl 4-(3-(4-bromo-2,5-difluorophenoxy)-2-oxopyrrolidin-1-yl)piperidine-1-carboxylate (5.6 g, 11.8 mmol) in DMSO (30 mL) was bubbled through with N₂ for 30 minutes. Trans-cyclohexane-1,2-diamine (0.538 g, 4.71 mmol), sodium methane sulfinate (1.68 g, 16.5 mmol) and Cu(I) triflate benzene complex (0.593 g, 1.18 mmol) were added and the reaction was stirred for 2 days at 100° C. The reaction was poured into water and extracted with EtOAc. The combined organic layers were washed with water and brine, dried over MgSO₄ and concentrated in vacuo. The crude material was purified over silica gel (15-100% EtOAc/CH₂Cl₂) to yield tert-butyl 4-(3-(2,5-difluoro-4-(methylsulfonyl)phenoxy)-2-oxopyrrolidin-1-yl)piperidine-1-carboxylate (2.3 g, 42% yield).

Step E:

To a solution of tert-butyl 4-(3-(2,5-difluoro-4-(methylsulfonyl)phenoxy)-2-oxopyrrolidin-1-yl)piperidine-1-carboxylate (2.3 g, 4.8 mmol) in CH₂Cl₂ was added trifluoroacetic acid (11 g, 97 mmol) and the reaction was stirred for 2 hours at ambient temperature. The reaction was concentrated in vacuo and the material partitioned between EtOAc and 1N NaOH. The layers were separated and the combined organic layers were dried over MgSO₄ and concentrated in vacuo to afford 3-(2,5-difluoro-4-(methylsulfonyl)phenoxy)-1-(piperidin-4-yl)pyrrolidin-2-one (1.5 g, 83% yield).

Step F:

To a solution of N-(methylsulfonyloxy)cyclopropanecarbimidoyl chloride (0.11 g, 0.56 mmol) in EtOAc (10 mL) was added sodium isothiocyanate (0.065 g, 0.80 mmol) and pyridine (0.13 g, 1.6 mmol). The reaction was stirred at 60° C. for 45 minutes. 3-(2,5-Difluoro-4-(methylsulfonyl)phenoxy)-1-(piperidin-4-yl)pyrrolidin-2-one (0.15 g, 0.40 mmol) was added and the reaction was heated to 60° C. overnight. The reaction was poured into water and extracted into EtOAc. The combined organic layers were washed with water, brine, dried over MgSO₄, filtered and concentrated in vacuo. The residue was purified over silica gel (100% EtOAc) to yield a solid, which was further purified by reverse phase HPLC (5 to 95% acetonitrile in water with 0.1% TFA) to yield 1-(1-(3-cyclopropyl-1,2,4-thiadiazol-5-yl)piperidin-4-yl)-3-(2,5-difluoro-4-(methylsulfonyl)phenoxy)pyrrolidin-2-one (0.028 g, 0.056 mmol, 14% yield). Mass Spectrum (apci) m/z=499.2 (M+H).

The following compounds were also prepared according to the method of Example 47.

| | | | Mass spectrum |
|---|---|---|---|
| Example | Structure | Name | (apci) |
| 48 | 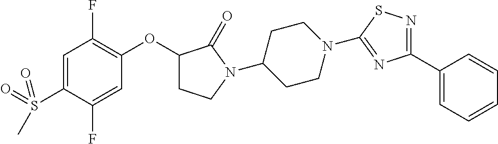 [SMILES: CS(=O)(=O)c1cc(F)c(OC2CCN(C3CCN(c4nc(-c5ccccc5)ns4)CC3)C2=O)cc1F] | 3-(2,5-difluoro-4- (methylsulfonyl)phenoxy)-1- (1-(3-phenyl-1,2,4-thiadiazol- 5-yl)piperidin-4-yl)pyrrolidin- 2-one trifluoroacetate | m/z = 535.2 (M + H). |
| 49 | 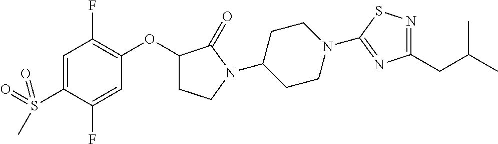 [SMILES: CC(C)Cc1nsc(N2CCC(N3CCC(Oc4cc(F)c(S(C)(=O)=O)cc4F)C3=O)CC2)n1] | 3-(2,5-difluoro-4- (methylsulfonyl)phenoxy)-1- (1-(3-isobutyl-1,2,4-thiadiazol- 5-yl)piperidin-4-yl)pyrrolidin- 2-one trifluoroacetate | m/z = 515.3 (M + H). |

### Example 50

### (S)-3-(2-fluoro-4-(methylsulfonyl)phenoxy)-1-(1-(3-isopropyl-1,2,4-thiadiazol-5-yl)piperidin-4-yl)pyrrolidin-2-one

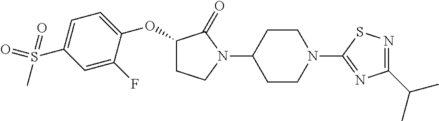 [SMILES: CC(C)c1nsc(N2CCC(N3CC[C@H](Oc4ccc(S(C)(=O)=O)cc4F)C3=O)CC2)n1]

Step A:

To a stirred suspension of (R)-3-hydroxydihydrofuran-2(3H)-one (3.0 g, 29 mmol) in toluene (200 mL) was added triphenylphosphine (9.2 g, 35 mmol) and 4-bromo-2-fluorophenol (6.7 g, 35 mmol). The mixture was cooled to 0° C. The solution was degassed with nitrogen for 10 minutes. Di-tert-butyl diazene-1,2-dicarboxylate (8.1 g, 35 mmol) was dissolved in toluene (50 mL) and added over 5 minutes to the reaction mixture. The reaction mixture was allowed to slowly warm to ambient temperature overnight. The reaction mixture was partitioned between water and EtOAc and extracted twice with EtOAc. The combined organic layers were dried over Na₂SO₄, filtered and concentrated. The product was purified over silica gel (25% EtOAc in hexanes) to afford (S)-3-(4-bromo-2-fluorophenoxy)dihydrofuran-2(3H)-one (5.2 g, 19 mmol, 64% yield) as a white solid.

Step B:

To a stirred solution of 1-(3-isopropyl-1,2,4-thiadiazol-5-yl)piperidin-4-amine (2.5 g, 11 mmol) in CH₂Cl₂ (20 mL) was added 1M trimethylaluminum in toluene (5.5 mL, 11 mmol) dropwise. The resulting mixture was stirred for 15 minutes. (S)-3-(4-Bromo-2-fluorophenoxy)dihydrofuran-2(3H)-one (2.5 g, 9.2 mmol) in CH₂Cl₂ (20 mL) was added slowly and the mixture was stirred at ambient temperature overnight. The reaction was slowly quenched with 5% tartaric acid and partitioned between saturated aqueous NaHCO₃ and EtOAc. The mixture was filtered and the filtrate was extracted with EtOAc. The combined organic layers were dried over Na₂SO₄, filtered and concentrated. The residue was purified over silica gel (100% EtOAc) to afford (S)-2-(4-bromo-2-fluorophenoxy)-4-hydroxy-N-(1-(3-isopropyl-1,2,4-thiadiazol-5-yl)piperidin-4-yl)butanamide (3.5 g, 7.0 mmol, 76% yield) as a white solid.

Step C:

Tributylphosphine (2.24 mL, 9.07 mmol) was added over 5 minutes to a degassed solution of di-tert-butyl diazene-1,2-dicarboxylate (2.09 g, 9.07 mmol) in dry THF (20 mL) at ambient temperature. The resulting mixture was stirred for 5 minutes, then added dropwise to a solution of (S)-2-(4-bromo-2-fluorophenoxy)-4-hydroxy-N-(1-(3-isopropyl-1,2,4-thiadiazol-5-yl)piperidin-4-yl)butanamide (3.5 g, 6.98 mmol) in THF (20 mL) at 0° C. and stirred for 2 hours. The reaction was partitioned between water and EtOAc and extracted with EtOAc. The combined organic layers were dried over Na₂SO₄, filtered and concentrated. The residue was purified over silica gel (70 to 90% EtOAc in hexanes) to afford (S)-3-(4-bromo-2-fluorophenoxy)-1-(1-(3-isopropyl-1,2,4-thiadiazol-5-yl)piperidin-4-yl)pyrrolidin-2-one (2.34 g, 4.84 mmol, 69.3% yield) as a white solid.

Step D:

(S)-3-(4-Bromo-2-fluorophenoxy)-1-(1-(3-isopropyl-1,2,4-thiadiazol-5-yl)piperidin-4-yl)pyrrolidin-2-one (2.34 g, 4.84 mmol), trans-cyclohexane-1,2-diamine (0.233 mL, 1.94 mmol), sodium methanesulfinate (0.741 g, 7.26 mmol) was dissolved in DMSO (40 mL) and nitrogen bubbled through the reaction mixture for 15 minutes. Cu(I) triflate benzene complex (0.244 g, 0.484 mmol) was added. The reaction was placed in a 110° C. oil bath and stirred under nitrogen for 8 hours. The reaction was partitioned between water and EtOAc and extracted with EtOAc. The combined organic layers were dried over Na₂SO₄, filtered and concentrated. The residue was purified over silica gel (2 to 5% methanol in EtOAc) to afford (S)-3-(2-fluoro-4-(methylsulfonyl)phenoxy)-1-(1-(3-isopropyl-1,2,4-thiadiazol-5-yl)piperidin-4-yl)pyrrolidin-2-one (1.99 g, 4.12 mmol, 85.2% yield) as a white solid. Mass spectrum (apci) m/z=483.2 (M+H).

The following compounds were also prepared according steps A-C or A-D of Example 50.

| | | | Mass spectrum |
|---|---|---|---|
| Example | Structure | Name | (apci) |
| 51 | 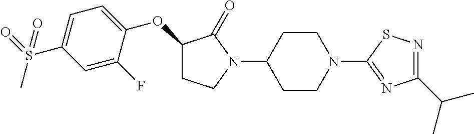 [SMILES: CC(C)c1nsc(N2CCC(N3CC[C@@H](Oc4ccc(S(C)(=O)=O)cc4F)C3=O)CC2)n1] | (R)-3-(2-fluoro-4- (methylsulfonyl)phenoxy)-1- (1-(3-isopropyl-1,2,4- thiadiazol-5-yl)piperidin-4- yl)pyrrolidin-2-one | m/z = 483.2 (M + H). |
| 52 | 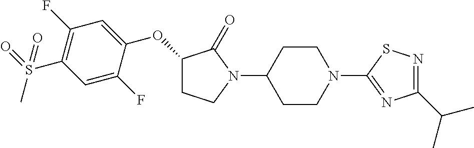 [SMILES: CC(C)c1nsc(N2CCC(N3CC[C@H](Oc4cc(F)c(S(C)(=O)=O)cc4F)C3=O)CC2)n1] | (S)-3-(2,5-difluoro-4- (methylsulfonyl)phenoxy)-1- (1-(3-isopropyl-1,2,4- thiadiazol-5-yl)piperidin-4- yl)pyrrolidin-2-one | m/z = 501.2 (M + H). |
| 53 | 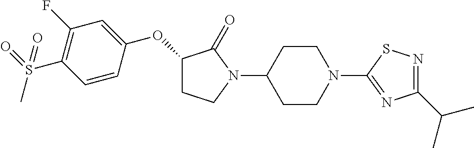 [SMILES: CC(C)c1nsc(N2CCC(N3CC[C@H](Oc4ccc(S(C)(=O)=O)c(F)c4)C3=O)CC2)n1] | (S)-3-(3-fluoro-4- (methylsulfonyl)phenoxy)-1- (1-(3-isopropyl-1,2,4- thiadiazol-5-yl)piperidin-4- yl)pyrrolidin-2-one | m/z = 483.2 (M + H). |
| 54 | 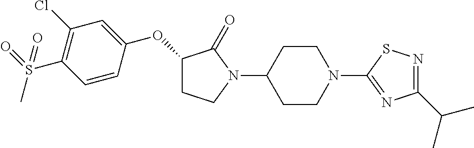 [SMILES: CC(C)c1nsc(N2CCC(N3CC[C@H](Oc4ccc(S(C)(=O)=O)c(Cl)c4)C3=O)CC2)n1] | (S)-3-(3-chloro-4- (methylsulfonyl)phenoxy)-1- (1-(3-isopropyl-1,2,4- thiadiazol-5-yl)piperidin-4- yl)pyrrolidin-2-one | m/z = 499.2 (M + H). |
| 55 | 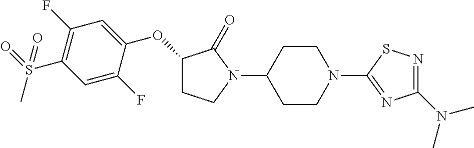 [SMILES: CN(C)c1nsc(N2CCC(N3CC[C@H](Oc4cc(F)c(S(C)(=O)=O)cc4F)C3=O)CC2)n1] | (S)-3-(2,5-difluoro-4- (methylsulfonyl)phenoxy)-1- (1-(3-(dimethylamino)-1,2,4- thiadiazol-5-yl)piperidin-4- yl)pyrrolidin-2-one | m/z = 502.2 (M + H). |
| 56 | 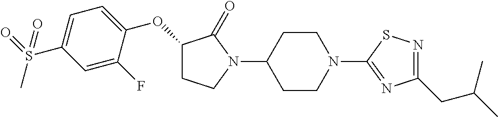 [SMILES: CC(C)Cc1nsc(N2CCC(N3CC[C@H](Oc4ccc(S(C)(=O)=O)cc4F)C3=O)CC2)n1] | (S)-3-(2-fluoro-4- (methylsulfonyl)phenoxy)-1- (1-(3-isobutyl-1,2,4-thiadiazol- 5-yl)piperidin-4-yl)pyrrolidin- 2-one | m/z = 497.2 (M + H). |
| 57 | 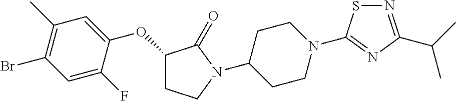 [SMILES: Cc1cc(O[C@H]2CCN(C3CCN(c4nc(C(C)C)ns4)CC3)C2=O)c(F)cc1Br] | (S)-3-(4-bromo-2-fluoro-5- methylphenoxy)-1-(1-(3- isopropyl-1,2,4-thiadiazol-5- yl)piperidin-4-yl)pyrrolidin-2- one | m/z = 497.2, 499.2 (M + H). |
| 58 | 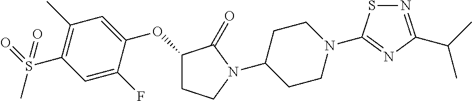 [SMILES: Cc1cc(O[C@H]2CCN(C3CCN(c4nc(C(C)C)ns4)CC3)C2=O)c(F)cc1S(C)(=O)=O] | (S)-3-(2-fluoro-5-methyl-4- (methylsulfonyl)phenoxy)-1- (1-(3-isopropyl-1,2,4- thiadiazol-5-yl)piperidin-4- yl)pyrrolidin-2-one | m/z = 497.3 (M + H). |
| 59 | 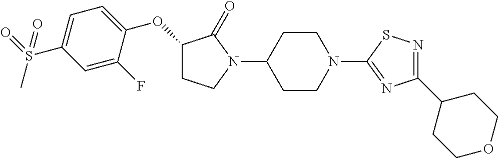 [SMILES: CS(=O)(=O)c1ccc(O[C@H]2CCN(C3CCN(c4nc(C5CCOCC5)ns4)CC3)C2=O)c(F)c1] | (S)-3-(2-fluoro-4- (methylsulfonyl)phenoxy)-1- (1-(3-(tetrahydro-2H-pyran-4- yl)-1,2,4-thiadiazol-5- yl)piperidin-4-yl)pyrrolidin-2- one | m/z = 525.2 (M + H). |
| 60 | 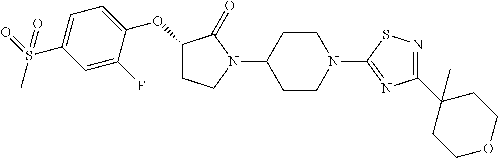 [SMILES: CC1(c2nsc(N3CCC(N4CC[C@H](Oc5ccc(S(C)(=O)=O)cc5F)C4=O)CC3)n2)CCOCC1] | (S)-3-(2-fluoro-4- (methylsulfonyl)phenoxy)-1- (1-(3-(4-methyltetrahydro-2H- pyran-4-yl)-1,2,4-thiadiazol-5- yl)piperidin-4-yl)pyrrolidin-2- one | m/z = 539.2 (M + H). |

### Example 61

### 3-(4-(ethylsulfonyl)-2-fluorophenoxy)-1-(1-(3-isopropyl-1,2,4-thiadiazol-5-yl)piperidin-4-yl)pyrrolidin-2-one

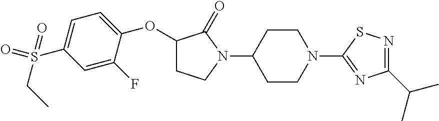 [SMILES: CCS(=O)(=O)c1ccc(OC2CCN(C3CCN(c4nc(C(C)C)ns4)CC3)C2=O)c(F)c1]

Step A:

In a sealed tube, 3-(4-bromo-2-fluorophenoxy)-1-(1-(3-isopropyl-1,2,4-thiadiazol-5-yl)piperidin-4-yl)pyrrolidin-2-one (Example 33; 200 mg, 0.414 mmol), Xantphos (23.9 mg, 0.0414 mmol), and N-ethyl-N-isopropylpropan-2-amine (216 μL, 1.24 mmol) were dissolved in dioxane (4 mL) and degassed with nitrogen for 5 minutes. Ethanethiol (61.3 μL, 0.827 mmol) and Pd₂ dba₃ (18.9 mg, 0.0207 mmol) were added. The mixture was placed in a 95° C. oil bath and stirred for 4 hours. The reaction was cooled, filtered and concentrated. The residue was purified over silica gel (80% EtOAc in hexanes) to afford 3-(4-(ethylthio)-2-fluorophenoxy)-1-(1-(3-isopropyl-1,2,4-thiadiazol-5-yl)piperidin-4-yl)pyrrolidin-2-one (188 mg, 0.405 mmol, 97.8% yield).

Step B:

3-(4-(Ethylthio)-2-fluorophenoxy)-1-(1-(3-isopropyl-1,2,4-thiadiazol-5-yl)piperidin-4-yl)pyrrolidin-2-one (188 mg, 0.405 mmol) was dissolved in CH₂Cl₂ (5 mL) and mCPBA (200 mg, 0.809 mmol) was added. The reaction was stirred at ambient temperature overnight. The reaction was partitioned between saturated aqueous NaHCO₃ and EtOAc, dried over Na₂SO₄, filtered and concentrated. The residue was purified over silica gel (100% EtOAc) to afford 3-(4-(ethylsulfonyl)-2-fluorophenoxy)-1-(1-(3-isopropyl-1,2,4-thiadiazol-5-yl)piperidin-4-yl)pyrrolidin-2-one (134 mg, 0.270 mmol, 66.7% yield) as a white solid. Mass spectrum (apci) m/z=497.2 (M+H).

### Example 62

### 3-(2-Fluoro-4-(phenylsulfonyl)phenoxy)-1-(1-(3-isopropyl-1,2,4-thiadiazol-5-yl)piperidin-4-yl)pyrrolidin-2-one

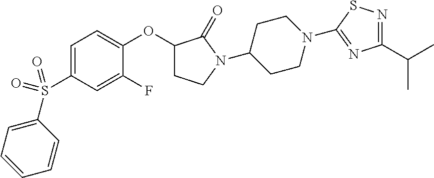 [SMILES: CC(C)c1nsc(N2CCC(N3CCC(Oc4ccc(S(=O)(=O)c5ccccc5)cc4F)C3=O)CC2)n1]

3-(4-Bromo-2-fluorophenoxy)-1-(1-(3-isopropyl-1,2,4-thiadiazol-5-yl)piperidin-4-yl)pyrrolidin-2-one (Example 33; 100 mg, 0.207 mmol) was dissolved in DMSO (2 mL) and nitrogen was bubbled through the reaction mixture for 15 minutes. Sodium benzenesulfinate (50.9 mg, 0.310 mmol), trans-cyclohexane-1,2-diamine (9.95 μL, 0.0827 mmol) and Cu(I) triflate benzene complex (10.4 mg, 0.0207 mmol) were added. The reaction was placed in a 110° C. oil bath and stirred under nitrogen overnight. The reaction was cooled, partitioned between water and EtOAc and extracted with EtOAc. The combined organic layers were dried over Na₂SO₄, filtered and concentrated. The residue was purified over silica gel (80% EtOAc in hexanes) to afford 3-(2-fluoro-4-(phenylsulfonyl)phenoxy)-1-(1-(3-isopropyl-1,2,4-thiadiazol-5-yl)piperidin-4-yl)pyrrolidin-2-one (84 mg, 0.154 mmol, 74.6% yield) as a white solid. Mass spectrum (apci) m/z=545.2 (M+H).

### Example 63

### 3-Fluoro-4-(1-(1-(3-isopropyl-1,2,4-thiadiazol-5-yl)piperidin-4-yl)-2-oxopyrrolidin-3-yloxy)benzonitrile

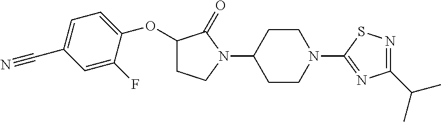 [SMILES: CC(C)c1nsc(N2CCC(N3CCC(Oc4ccc(C#N)cc4F)C3=O)CC2)n1]

3-(4-Bromo-2-fluorophenoxy)-1-(1-(3-isopropyl-1,2,4-thiadiazol-5-yl)piperidin-4-yl)pyrrolidin-2-one (Example 33; 300 mg, 0.621 mmol) was dissolved in NMP (1 mL). Cu(I)CN (222 mg, 2.48 mmol) was added and the mixture was heated to 160° C. overnight. The reaction was cooled to ambient temperature, diluted with water, filtered, extracted with EtOAc, dried over Na₂SO₄, filtered and concentrated. The residue was purified over silica gel to afford 3-fluoro-4-(1-(1-(3-isopropyl-1,2,4-thiadiazol-5-yl)piperidin-4-yl)-2-oxopyrrolidin-3-yloxy)benzonitrile (66 mg, 0.154 mmol, 24.8% yield) as a tan solid. Mass spectrum (apci) m/z=430.2 (M+H).

The following compound was prepared according to the procedure for Example 63.

| | | | Mass spectrum |
|---|---|---|---|
| Example | Structure | Name | (apci) |
| 64 | 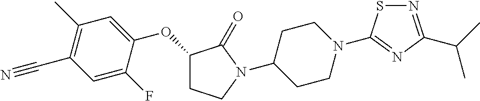 [SMILES: Cc1cc(O[C@H]2CCN(C3CCN(c4nc(C(C)C)ns4)CC3)C2=O)c(F)cc1C#N] | (S)-5-fluoro-4-(1-(1-(3- isopropyl-1,2,4-thiadiazol-5- yl)piperidin-4-yl)-2- oxopyrrolidin-3-yloxy)-2- methylbenzonitrile | m/z = 444.2 (M + H). |

### Example 65

### -(1-(3-Isopropyl-1,2,4-thiadiazol-5-yl)piperidin-4-yl)-3-(4-(methylsulfonyl)phenoxy)pyrrolidin-2-one

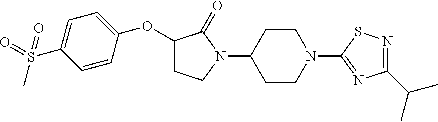 [SMILES: CC(C)c1nsc(N2CCC(N3CCC(Oc4ccc(S(C)(=O)=O)cc4)C3=O)CC2)n1]

1-(1-(3-Isopropyl-1,2,4-thiadiazol-5-yl)piperidin-4-yl)-3-(4-(methylthio)phenoxy)pyrrolidin-2-one (Example 32; 122 mg, 0.282 mmol) was dissolved in CH₂Cl₂ (5 mL) and 70% mCPBA (153 mg, 0.620 mmol) was added. After 1 hour the reaction was partitioned between saturated aqueous NaHCO₃ and CH₂Cl₂, extracted with CH₂Cl₂, dried over Na₂SO₄, filtered and concentrated. The residue was purified over silica gel (90 to 100% EtOAc in hexanes) to afford 1-(1-(3-isopropyl-1,2,4-thiadiazol-5-yl)piperidin-4-yl)-3-(4-(methylsulfonyl)phenoxy)pyrrolidin-2-one (79.2 mg, 0.170 mmol, 60.4% yield) as a white solid. Mass spectrum (apci) m/z=465.2 (M+H).

### Example 66

### 3-(4-(ethylsulfonyl)-2-fluorophenoxy)-1-(1-(3-methyl-1,2,4-thiadiazol-5-yl)piperidin-4-yl)pyrrolidin-2-one

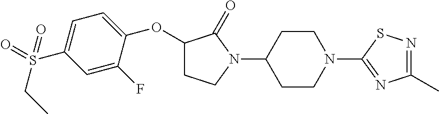 [SMILES: CCS(=O)(=O)c1ccc(OC2CCN(C3CCN(c4nc(C)ns4)CC3)C2=O)c(F)c1]

Step A:

Tert-butyl 4-aminopiperidine-1-carboxylate (10 g, 49.9 mmol) and triethylamine (7.66 mL, 54.9 mmol) were dissolved in THF (250 mL). 2,4-Dibromobutanoyl chloride (6.60 mL, 49.9 mmol) was added slowly, and the mixture was stirred at ambient temperature for 30 minutes. The resultant solids were filtered and the filtrate was concentrated to afford crude tert-butyl 4-(2,4-dibromobutanamido)piperidine-1-carboxylate (21.4 g, 50.0 mmol, 100% yield).

Step B:

Tert-butyl 4-(2,4-dibromobutanamido)piperidine-1-carboxylate (21.4 g, 50.0 mmol) was dissolved in DMF (250 mL). Sodium hydride (2.00 g, 50.0 mmol) was added and the reaction was stirred at ambient temperature for 2 hours. The reaction was concentrated in vacuo, partitioned between aqueous NH₄Cl and EtOAc, dried over Na₂SO₄, filtered and concentrated. The residue was purified over silica gel to afford tert-butyl 4-(3-bromo-2-oxopyrrolidin-1-yl)piperidine-1-carboxylate (12.5 g, 36.0 mmol, 72.0% yield).

Step C:

Tert-butyl 4-(3-bromo-2-oxopyrrolidin-1-yl)piperidine-1-carboxylate (12.2 g, 35.1 mmol) and 4-bromo-2-fluorophenol (10.1 g, 52.7 mmol) were dissolved in DMF (200 mL). K₂CO₃ (5.34 g, 38.6 mmol) was added and the reaction was heated to 50° C. for 2 hours. The reaction was filtered and concentrated in vacuo. The crude material was purified over silica gel (70% EtOAc in hexanes) to afford tert-butyl 4-(3-(4-bromo-2-fluorophenoxy)-2-oxopyrrolidin-1-yl)piperidine-1-carboxylate (11.2 g, 24.5 mmol, 69.7% yield) as an amber oil that slowly solidified.

Step D:

tert-Butyl 4-(3-(4-bromo-2-fluorophenoxy)-2-oxopyrrolidin-1-yl)piperidine-1-carboxylate (11.2 g, 24.5 mmol) was dissolved in dioxane (200 mL) in a 350 mL pressure flask. Xantphos (0.709 g, 1.22 mmol) and N-ethyl-N-isopropylpropan-2-amine (8.53 mL, 49.0 mmol) were added. Nitrogen was bubbled through the solution for 10 minutes. Pd₂ dba₃ (0.561 g, 0.612 mmol) and ethanethiol (1.99 mL, 26.9 mmol) were added and the flask was sealed and placed in 95° C. oil bath for 3 hours. The reaction was cooled to ambient temperature, and the resultant precipitate was filtered. The filtrate was concentrated and purified over silica gel (50% EtOAc in hexanes) to afford tert-butyl 4-(3-(4-(ethylthio)-2-fluorophenoxy)-2-oxopyrrolidin-1-yl)piperidine-1-carboxylate (8.3 g, 18.9 mmol, 77.3% yield).

Step E:

tert-Butyl 4-(3-(4-(ethylthio)-2-fluorophenoxy)-2-oxopyrrolidin-1-yl)piperidine-1-carboxylate (8.3 g, 19 mmol) was dissolved in CH₂Cl₂ (200 mL) and mCPBA (9.8 g, 40 mmol) was added. The reaction was stirred at ambient temperature for 1 hour. The reaction was cooled on an ice bath to precipitate out most of carboxylic acid. The mixture was filtered into saturated aqueous NaHCO₃, extracted with CH₂Cl₂, dried over Na₂SO₄, filtered and concentrated to afford tert-butyl 4-(3-(4-(ethylsulfonyl)-2-fluorophenoxy)-2-oxopyrrolidin-1-yl)piperidine-1-carboxylate (9.0 g, 19 mmol, 101% yield) as a pale yellow solid.

Step F:

Tert-butyl 4-(3-(4-(ethylsulfonyl)-2-fluorophenoxy)-2-oxopyrrolidin-1-yl)piperidine-1-carboxylate (9.0 g, 19 mmol) was dissolved in CH₂Cl₂ (50 mL), 4N HCl in dioxane (50 mL) was added and the reaction mixture was stirred at ambient temperature for 30 minutes. The reaction was concentrated and dried on high vacuum to afford 3-(4-(ethylsulfonyl)-2-fluorophenoxy)-1-(piperidin-4-yl)pyrrolidin-2-one hydrochloride (7.6 g, 19 mmol, 98% yield) as a yellow solid.

Step G:

N-(Methylsulfonyloxy)acetimidoyl chloride (94.9 mg, 0.553 mmol) was dissolved in CH₃CN (4 mL). Pyridine (148 μL, 1.84 mmol) and NaNCS (44.8 mg, 0.553 mmol) were added. The reaction was heated to 45° C. for 45 minutes. 3-(4-(Ethylsulfonyl)-2-fluorophenoxy)-1-(piperidin-4-yl)pyrrolidin-2-one hydrochloride (150 mg, 0.369 mmol) was added and the reaction was heated at 45° C. for 45 minutes. The reaction was cooled to ambient temperature, partitioned between water and EtOAc, dried over Na₂SO₄, filtered and concentrated. The residue was purified over silica gel (5% methanol in EtOAc) to afford 3-(4-(ethylsulfonyl)-2-fluorophenoxy)-1-(1-(3-methyl-1,2,4-thiadiazol-5-yl)piperidin-4-yl)pyrrolidin-2-one (85 mg, 0.181 mmol, 49.2% yield) as a pale yellow solid. Mass spectrum (apci) m/z=469.2 (M+H).

The following compounds were prepared according to the method of Example 66.

| | | | Mass spectrum |
|---|---|---|---|
| Example | Structure | Name | (apci) |
| 67 | 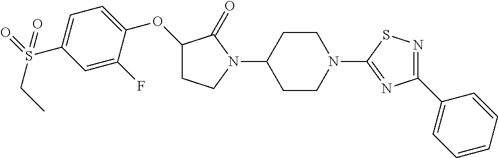 [SMILES: CCS(=O)(=O)c1ccc(OC2CCN(C3CCN(c4nc(-c5ccccc5)ns4)CC3)C2=O)c(F)c1] | 3-(4-(ethylsulfonyl)-2- fluorophenoxy)-1-(1-(3- phenyl-1,2,4-thiadiazol-5- yl)piperidin-4-yl)pyrrolidin-2- one | m/z = 531.2 (M + H). |
| 68 | 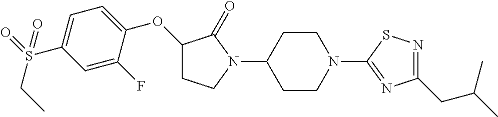 [SMILES: CCS(=O)(=O)c1ccc(OC2CCN(C3CCN(c4nc(CC(C)C)ns4)CC3)C2=O)c(F)c1] | 3-(4-(ethylsulfonyl)-2- fluorophenoxy)-1-(1-(3- isobutyl-1,2,4-thiadiazol-5- yl)piperidin-4-yl)pyrrolidin-2- one | m/z = 511.2 (M + H). |

### Example 69

### (S)-1-(1-(3-bromo-1,2,4-thiadiazol-5-yl)piperidin-4-yl)-3-(2-fluoro-4-(methylsulfonyl)phenoxy)pyrrolidin-2-one

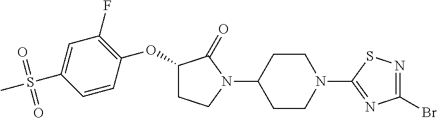 [SMILES: CS(=O)(=O)c1ccc(O[C@H]2CCN(C3CCN(c4nc(Br)ns4)CC3)C2=O)c(F)c1]

To a mixture of (S)-3-(2-fluoro-4-(methylsulfonyl)phenoxy)-1-(piperidin-4-yl)pyrrolidin-2-one hydrochloride (Preparation II; 0.358 g, 0.911 mmol) in THF (10 mL) was added triethylamine (0.286 mL, 2.05 mmol) and the mixture was stirred at ambient temperature. A solution of 3-bromo-5-chloro-1,2,4-thiadiazole (0.200 g, 1.00 mmol) in THF (5 mL) was added, and the reaction was stirred overnight at ambient temperature. The mixture was diluted with EtOAc and washed with water. The combined organic layers were dried over Na₂SO₄ and concentrated to afford (S)-1-(1-(3-bromo-1,2,4-thiadiazol-5-yl)piperidin-4-yl)-3-(2-fluoro-4-(methylsulfonyl)phenoxy)pyrrolidin-2-one (0.493 g, 0.949 mmol, 104% yield) as a white solid. Mass spectrum (apci) m/z=519.0, 521.1 (M+H).

### Example 70

### (S)-3-(2-fluoro-4-(methylsulfonyl)phenoxy)-1-(1-(3-(oxazol-5-yl)-1,2,4-thiadiazol-5-yl)piperidin-4-yl)pyrrolidin-2-one

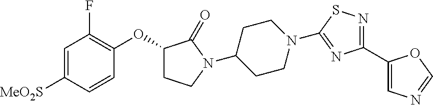 [SMILES: CS(=O)(=O)c1ccc(O[C@H]2CCN(C3CCN(c4nc(-c5cnco5)ns4)CC3)C2=O)c(F)c1]

A mixture of CsF (0.0461 g, 0.303 mmol), (S)-1-(1-(3-bromo-1,2,4-thiadiazol-5-yl)piperidin-4-yl)-3-(2-fluoro-4-(methylsulfonyl)phenoxy)pyrrolidin-2-one (Example 69; 0.075 g, 0.144 mmol), 5-(4,4,5,5-tetramethyl-1,3,2-dioxaborolan-2-yl)oxazole (0.042 g, 0.217 mmol) and bis(di-tert-butyl(4-dimethylaminophenyl)phosphine)dichloropalladium (II) (0.020 g, 0.0289 mmol) was degassed with nitrogen and 10% aqueous dioxane (2 mL) was added. The reaction was heated at 80° C. overnight. The reaction was cooled to ambient temperature and partitioned between EtOAc and 1 N NaOH. The combined organic layers were washed with water and brine, dried over Na₂SO₄, filtered and concentrated. The residue was purified by preparative HPLC (Parallex Flex) to give (S)-3-(2-fluoro-4-(methylsulfonyl)phenoxy)-1-(1-(3-(oxazol-5-yl)-1,2,4-thiadiazol-5-yl)piperidin-4-yl)pyrrolidin-2-one (0.003 g, 4.09% yield). Mass spectrum (apci) m/z=508.2 (M+H).

The following compounds were prepared according to the method of Example 70.

| | | | Mass spectrum |
|---|---|---|---|
| Example | Structure | Name | (apci) |
| 71 | 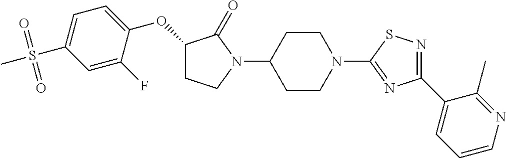 [SMILES: Cc1ncccc1-c1nsc(N2CCC(N3CC[C@H](Oc4ccc(S(C)(=O)=O)cc4F)C3=O)CC2)n1] | (S)-3-(2-fluoro-4- (methylsulfonyl)phenoxy)-1- (1-(3-(2-methylpyridin-3-yl)- 1,2,4-thiadiazol-5- yl)piperidin-4-yl)pyrrolidin-2- one | m/z = 532.2 (M + H). |
| 72 | 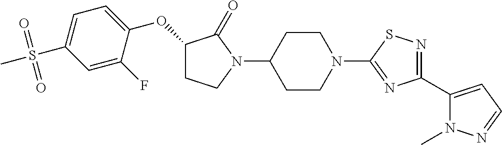 [SMILES: Cn1nccc1-c1nsc(N2CCC(N3CC[C@H](Oc4ccc(S(C)(=O)=O)cc4F)C3=O)CC2)n1] | (S)-3-(2-fluoro-4- (methylsulfonyl)phenoxy)-1- (1-(3-(1-methyl-1H-pyrazol- 5-yl)-1,2,4-thiadiazol-5- yl)piperidin-4-yl)pyrrolidin-2- one | m/z = 521.2 (M + H). |
| 73 | 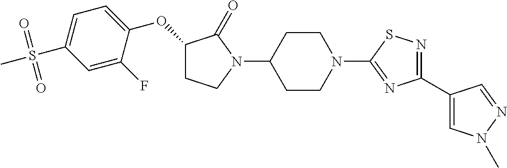 [SMILES: Cn1cc(-c2nsc(N3CCC(N4CC[C@H](Oc5ccc(S(C)(=O)=O)cc5F)C4=O)CC3)n2)cn1] | (S)-3-(2-fluoro-4- (methylsulfonyl)phenoxy)-1- (1-(3-(1-methyl-1H-pyrazol- 4-yl)-1,2,4-thiadiazol-5- yl)piperidin-4-yl)pyrrolidin-2- one | m/z = 521.1 (M + H). |

### Example 74

### (S)-3-(2-fluoro-4-(methylsulfonyl)phenoxy)-1-(1-(3-phenoxy-1,2,4-thiadiazol-5-yl)piperidin-4-yl)pyrrolidin-2-one

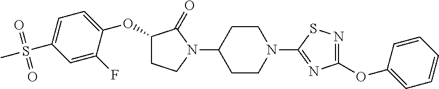 [SMILES: CS(=O)(=O)c1ccc(O[C@H]2CCN(C3CCN(c4nc(Oc5ccccc5)ns4)CC3)C2=O)c(F)c1]

To a mixture of (S)-1-(1-(3-bromo-1,2,4-thiadiazol-5-yl)piperidin-4-yl)-3-(2-fluoro-4-(methylsulfonyl)phenoxy)pyrrolidin-2-one (Example 69; 0.200 g, 0.385 mmol), phenol (0.047 g, 0.501 mmol) and Cs₂CO₃ (0.376 g, 1.16 mmol) was added DMSO (2 mL). The mixture was stirred at 110° C. overnight. The reaction was cooled to ambient temperature and partitioned between EtOAc and water. The combined organic layers were dried over Na₂SO₄, filtered and concentrated. The residue was purified by preparative HPLC (Parrallex Flex) to give (S)-3-(2-fluoro-4-(methylsulfonyl)phenoxy)-1-(1-(3-phenoxy-1,2,4-thiadiazol-5-yl)piperidin-4-yl)pyrrolidin-2-one (0.073 g, 35.6% yield). Mass spectrum (apci) m/z=533.2 (M+H).

The following compounds were prepared according to the method of Example 74.

| | | | Mass spectrum |
|---|---|---|---|
| Example | Structure | Name | (apci) |
| 75 | 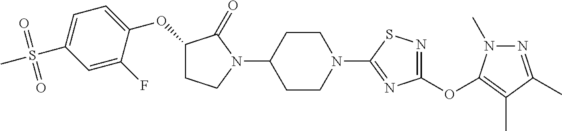 | (S)-3-(2-fluoro-4- (methylsulfonyl)phenoxy)- 1-(1-(3-(1,3,4-trimethyl-1H- pyrazol-5-yloxy)-1,2,4- thiadiazol-5-yl)piperidin-4- yl)pyrrolidin-2-one | m/z = 565.2 (M + H). |
| 76 | 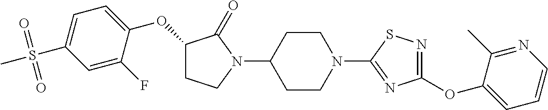 [SMILES: Cc1ncccc1Oc1nsc(N2CCC(N3CC[C@H](Oc4ccc(S(C)(=O)=O)cc4F)C3=O)CC2)n1] | (S)-3-(2-fluoro-4- (methylsulfonyl)phenoxy)- 1-(1-(3-(2-methylpyridin-3- yloxy)-1,2,4-thiadiazol-5- yl)piperidin-4-yl)pyrrolidin- 2-one | m/z = 548.2 (M + H). |

### Example 77

### 3-Fluoro-N-(2-hydroxyethyl)-4-(1-(1-(3-isopropyl-1,2,4-thiadiazol-5-yl)piperidin-4-yl)-2-oxopyrrolidin-3-yloxy)benzamide hydrochloride

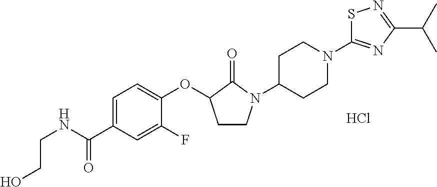 [SMILES: CC(C)c1nsc(N2CCC(N3CCC(Oc4ccc(C(=O)NCCO)cc4F)C3=O)CC2)n1 ; Cl]

Step A:

To a solution of tert-butyl-4-(3-bromo-2-oxopyrrolidin-1-yl)piperidine-1-carboxylate (750 mg, 2.16 mmol), and methyl-3-fluoro-4-hydroxybenzoate

(441 mg, 2.59 mmol) in DMSO (4 mL) was added K₂CO₃ (328 mg, 2.38 mmol). The reaction was heated to 60° C. overnight. The reaction mixture was diluted with water (5 mL) and EtOAc (20 mL). The organic layer was separated and the aqueous layer washed with EtOAc. The combined organics layers were dried over MgSO₄ and concentrated. The crude material was purified over silica gel (eluting with a gradient of 4:1 up to 2:1 hexanes/EtOAc) to give tert-butyl-4-(3-(2-fluoro-4-(methoxycarbonyl)phenoxy)-2-oxopyrrolidin-1-yl)piperidine-1-carboxylate (300 mg, 32%) as a light yellow solid. The solid material was dissolved into DCM (15 mL) and excess 4N HCl in dioxane (5 mL) added. The reaction was allowed to stir overnight and concentrated to give methyl 3-fluoro-4-(2-oxo-1-(piperidin-4-yl)pyrrolidin-3-yloxy)benzoate hydrochloride (190 mg, 89%) as a light tan solid.

Step B:

To a suspension of N-methylsulfonlyoxy)isobutyrimidoyl chloride (122 mg, 0.612 mmol), and sodium isothiocyanate (49.6 mg, 0.612 mmol) in EtOAc (5 mL) was added pyridine (403 mg, 5.10 mmol). The reaction was heated to 60° C. for 45 minutes. 3-Fluoro-4-(2-oxo-1-(piperidin-4-yl)pyrrolidin-3-yloxy)benzoate HCl salt (190 mg, 0.510 mmol) was added and the reaction temperature was maintained at 60° C. overnight. The reaction was partitioned between water (5 mL) and EtOAc (10 mL). The organic layer was separated and the aqueous layer washed with EtOAc (2×10 mL). The combined organics were dried over MgSO₄, filtered and concentrated. The crude material was purified over silica gel (4:1-2:1 hexanes/EtOAc) to give methyl 3-fluoro-4-(1-(1-(3-isopropyl-1,2,4-thiadiazol-5-yl)piperidin-4-yl)-2-oxopyrrolidin-3-yloxy)benzoate (200 mg, 85%) as an off white foam.

Step C:

To a solution of methyl 3-fluoro-4-(1-(1-(3-isopropyl-1,2,4-thiadiazol-5-yl)piperidin-4-yl)-2-oxopyrrolidin-3-yloxy)benzoate (200 mg, 0.432 mmol) in a mixture of THF/MeOH/H₂O (5/2/2 mL) was added NaOH (20.0 mg, 0.50 mmol) and the reaction allowed to stir at ambient temperature overnight. The reaction was concentrated and then diluted with EtOAc (10 mL) and 1N aqueous HCl (2 mL). The organic layer was separated and the aqueous layer washed with EtOAc (2×5 mL). The combined organic layers were dried over MgSO₄, filtered and concentrated to give 3-fluoro-4-(1-(1-(3-isopropyl-1,2,4-thiadiazol-5-yl)piperidin-4-yl)-2-oxopyrrolidin-3-yloxy)benzoic acid (191 mg, 98%) as an off white solid which was used without further purification.

Step D:

To a solution of 3-fluoro-4-(1-(1-(3-isopropyl-1,2,4-thiadiazol-5-yl)piperidin-4-yl)-2-oxopyrrolidin-3-yloxy)benzoic acid (22 mg, 0.049 mmol), and HATU (20.5 mg, 0.054 mmol) in CH₂Cl₂ (4 mL) was added a solution of 2-aminoethanol (3.3 mg, 0.054 mmol) in CH₂Cl₂ (1 mL). The reaction was allowed to stir overnight at ambient temperature and then diluted with water (5 mL). The organic layer was separated and the aqueous layer washed with CH₂Cl₂ (2×5 mL). The combined organic layers were dried over MgSO₄, filtered and concentrated. The crude material was purified over silica gel (33 to 100% EtOAc in hexanes) to give 3-fluoro-N-(2-hydroxyethyl)-4-(1-(1-(3-isopropyl-1,2,4-thiadiazol-5-yl)piperidin-4-yl)-2-oxopyrrolidin-3-yloxy)benzamide (9 mg, 37%) as a clear oil. The oil was dissolved into CH₂Cl₂ (3 mL) and HCl (2N in ether) was added. The mixture was concentrated to give 3-Fluoro-N-(2-hydroxyethyl)-4-(1-(1-(3-isopropyl-1,2,4-thiadiazol-5-yl)piperidin-4-yl)-2-oxopyrrolidin-3-yloxy)benzamide hydrochloride. Mass spectrum (apci) m/z=492.2 (M+H).

The following compounds were also prepared according to the procedure for Example 77.

| | | | Mass |
|---|---|---|---|
| | | | Spectrum |
| Example | Structure | Name | (apci) |
| 78 | 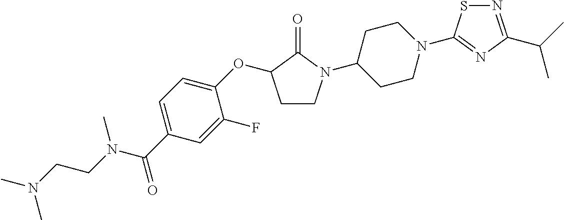 [SMILES: CC(C)c1nsc(N2CCC(N3CCC(Oc4ccc(C(=O)N(C)CCN(C)C)cc4F)C3=O)CC2)n1] | N-(2- (dimethylamino)ethyl)-3- fluoro-4-(1-(1-(3- isopropyl-1,2,4- thiadiazol-5-yl)piperidin- 4-yl)-2-oxopyrrolidin-3- yloxy)-N- methylbenzamide | m/z = 533.2 (M + H). |
| 79 | 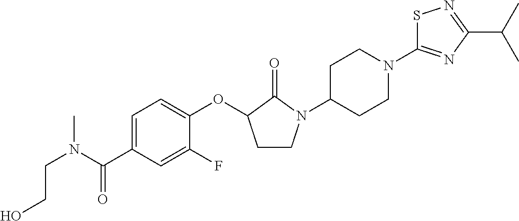 [SMILES: CC(C)c1nsc(N2CCC(N3CCC(Oc4ccc(C(=O)N(C)CCO)cc4F)C3=O)CC2)n1] | 3-fluoro-N-(2- hydroxyethyl)-4-(1-(1-(3 isopropyl-1,2,4- thiadiazol-5-yl)piperidin- 4-yl)-2-oxopyrrolidin-3- yloxy)-N- methylbenzamide | m/z = 506.2 (M + H). |
| 80 | 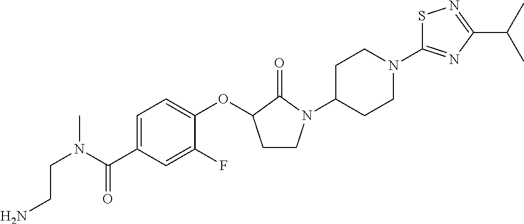 [SMILES: CC(C)c1nsc(N2CCC(N3CCC(Oc4ccc(C(=O)N(C)CCN)cc4F)C3=O)CC2)n1] | N-(2-aminoethyl)-3- fluoro-4-(1-(1-(3- isopropyl-1,2,4- thiadiazol-5-yl)piperidin- 4-yl)-2-oxopyrrolidin-3- yloxy)-N- methylbenzamide | m/z = 505.2 (M + H). |
| 81 | 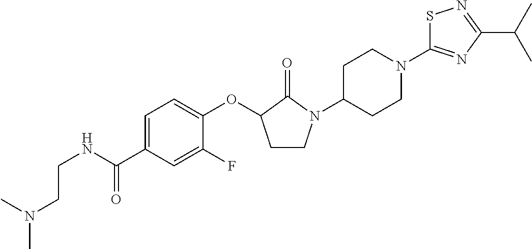 [SMILES: CC(C)c1nsc(N2CCC(N3CCC(Oc4ccc(C(=O)NCCN(C)C)cc4F)C3=O)CC2)n1] | N-(2- (dimethylamino)ethyl)-3 fluoro-4-(1-(1-(3- isopropyl-1,2,4- thiadiazol-5-yl)piperidin- 4-yl)-2-oxopyrrolidin-3- yloxy)benzamide | m/z = 519.3 (M + H). |

### Example 82

### 3-(6-(1H-tetrazol-1-yl)pyridin-3-yloxy)-1-(1-(3-isopropyl-1,2,4-thiadiazol-5-yl)piperidin-4-yl)pyrrolidin-2-one

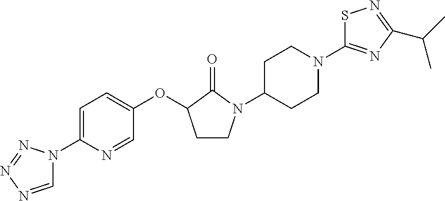 [SMILES: CC(C)c1nsc(N2CCC(N3CCC(Oc4ccc(-n5cnnn5)nc4)C3=O)CC2)n1]

Step A:

To a solution of sodium azide (2.90 g, 44 mmol) in triethylorthoformate (8 mL) and AcOH (50 mL) was added 6-amino-pyridine-3-ol (3.5 g, 32 mmol). The reaction was heated to 100° C. for 6 hours and then cooled to ambient temperature and allowed to stir overnight. The solids were filtered, washed with EtOAc and dried in vacuo to give 6-(1H-tetrazol-1-yl)pyridin-3-ol (4.20 g, 81%) as a beige solid.

Step B:

To a solution of tert-butyl 4(3-bromo-2-oxopyrrolidine-1-yl)piperidine-1-carboxylate (1.04 g, 3.0 mmol) and 6-(1H-tetrazol-1-yl)pyridin-3-ol (538 mg, 3.3 mmol) in DMSO (10 mL) was added K₂CO₃ (1.04 g, 7.5 mmol). The reaction was heated to 70° C. for 12 hours. The reaction was cooled to ambient temperature and diluted with water (10 mL) and EtOAc (20 mL). The organic layer was separated and the aqueous layer extracted with EtOAc (2×15 mL). The combined organics were dried over MgSO₄ and concentrated in vacuo. The material was purified by silica gel chromatography eluting Hexanes/EtOAc (3:1) to yield tert-butyl 4(3-(6-(1H-tetrazol-1-yl)pyridine-3-yloxy-2-oxopyrrolidin-1-yl)piperidine-1-carboxylate (978 mg, 76%) as an off white solid.

Step C:

To a solution of tert-butyl 4(3-(6-(1H-tetrazol-1-yl)pyridine-3-yloxy-2-oxopyrrolidin-1-yl)piperidine-1-carboxylate (978 mg, 2.28 mmol) in DCM (10 mL) was added excess 4N HCl in dioxane (3 mL). The reaction was stirred for 4 hours and then concentrated in vacuo to yield 3-(6-(1H-tetrazol-1-yl)pyridin-3-yloxy)-1-(piperidin-4-yl)pyrrolidin-2-one bishydrochloride salt (796 mg, 87%) as an off white solid, which was used without any further purification.

Step D:

To a solution of N-(methylsulfonyloxy)isobutyrimidoyl chloride (62 mg, 0.31 mmol) in EtOAc/CH₃CN (3:1 mL) was added NaNCS (25 mg, 0.31 mmol) and pyridine (204 mg, 2.6 mmol). The reaction was heated to 60° C. for 45 minutes. 3-(6-(1H-Tetrazol-1-yl)pyridin-3-yloxy)-1-(piperidin-4-yl)pyrrolidin-2-one bis-hydrochloride salt (104 mg, 0.26 mmol) was added and the reaction was allowed to stir at 60° C. for 10 hours. The reaction was quenched by the addition of water (5 mL) and EtOAc (5 mL). The organic layer was separated and the aqueous layer was extracted with EtOAc. The combined organic layers were dried over MgSO₄, filtered and concentrated in vacuo. The resulting residue was purified by silica gel chromatography, eluting with Hexanes/EtOAc (1:1)—100% EtOAc to give 3-(6-(1H-tetrazol-1-yl)pyridine-3-yloxy)-1-(1-(3-isopropyl-1,2,4-thiadiazol-5-yl)piperidin-4-yl)pyrrolidin-2-one (88 mg, 75%) as an off white solid. Mass spectrum (apci) m/z=456.0 M+H).

### Example 83

### 1-(1-(3-isopropyl-1,2,4-thiadiazol-5-yl)piperidin-4-yl)-3-(4-(2-methyl-2H-tetrazol-5-yl)phenoxy)pyrrolidin-2-one

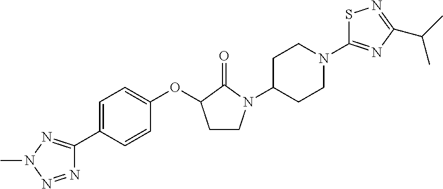 [SMILES: CC(C)c1nsc(N2CCC(N3CCC(Oc4ccc(-c5nnn(C)n5)cc4)C3=O)CC2)n1]

Step A:

To a solution of tert-butyl 4-(3-bromo-2-oxopyrrolidin-1-yl)piperidine-1-carboxylate (650 mg, 1.87 mmol) and 4-(2-methyl-2H-tetrazol-5-yl)phenol (330 mg, 1.87 mmol) in DMSO (10 mL) was added K₂CO₃ (647 mg, 4.68 mmol). The reaction was heated to 70° C. for 10 hours. The reaction was quenched by the addition of water (10 mL) and EtOAc (10 mL). The organic layer was separated and the aqueous layer extracted with EtOAc (2×15 mL). The combined organic layers were dried over MgSO₄ and concentrated in vacuo. The resulting residue was purified by silica gel chromatography, eluting with EtOAc, to give tert-butyl 4-(3-(4-(2-methyl-2H-tetrazol-5-yl)phenoxy)-2-oxopyrrolidin-1-yl)piperidine-1-carboxylate (300 mg, 36%) as a clear oil.

Step B:

To a solution of tert-butyl 4-(3-(4-(2-methyl-2H-tetrazol-5-yl)phenoxy)-2-oxopyrrolidin-1-yl)piperidine-1-carboxylate (300 mg, 0.68 mmol) in CH₂Cl₂ (10 mL) was added excess 4 N HCl in dioxane (3 mL). The reaction was allowed to stir for 4 hours and then concentrated in vacuo to give 3-(4-(2-methyl-2H-tetrazol-5-yl)phenoxy)-1-(piperidin-4-yl)pyrrolidin-2-one hydrochloride salt (248 mg, 97%) as an off white solid.

Step C:

To a solution of N-(methylsulfonyloxy)isobutyrimidoyl chloride (48 mg, 0.24 mmol) in EtOAc/CH₃CN (3:1 mL) was added NaNCS (15 mg, 0.19 mmol) and pyridine (146 mg, 1.9 mmol). The reaction was heated to 60° C. for 45 minutes. 3-(6-(1H-Tetrazol-1-yl)pyridin-3-yloxy)-1-(piperidin-4-yl)pyrrolidin-2-one bishydrochloride salt (104 mg, 0.26 mmol) was added and the reaction allowed to stir at 60° C. for 10 hours. The reaction was quenched by the addition of water (5 mL) and EtOAc (5 mL). The organic layer was separated and the aqueous layer was extracted with EtOAc (2×5 mL). The combined organic layers were dried over MgSO₄, filtered and concentrated in vacuo. The resulting residue was purified by silica gel chromatography, eluting with Hexanes/EtOAc (1:1)—100% EtOAc to give 1-(1-(3-isopropyl-1,2,4-thiadiazol-5-yl)piperidin-4-yl)-3-(4-(2-methyl-2H-tetrazol-5-yl)phenoxy)pyrrolidin-2-one (56 mg, 65%) as an off white solid. Mass spectrum (apci) m/z=469.2 (M+H).

The following compound was also prepared according to procedure of Example 83.

| | | | Mass spectrum |
|---|---|---|---|
| Example | Structure | Name | (apci) |
| 84 | 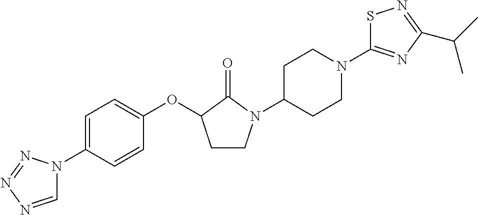 [SMILES: CC(C)c1nsc(N2CCC(N3CCC(Oc4ccc(-n5cnnn5)cc4)C3=O)CC2)n1] | 3-(4-(1H-tetrazol-1- yl)phenoxy)-1-(1-(3- isopropyl-1,2,4-thiadiazol-5- yl)piperidin-4-yl)pyrrolidin- 2-one | m/z = 455.1 (M + H). |

### Example 85

### 3-(2,5-Difluoro-4-(methylsulfonyl)phenoxy)-1′-(3-isopropyl-1,2,4-thiadiazol-5-yl)-[1,4′-bipiperidin]-2-one

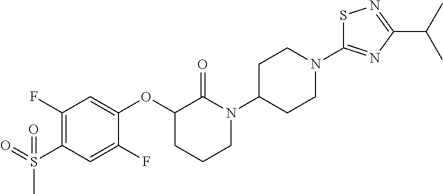 [SMILES: CC(C)c1nsc(N2CCC(N3CCCC(Oc4cc(F)c(S(C)(=O)=O)cc4F)C3=O)CC2)n1]

Step A:

To a 3-neck flask equipped with an addition funnel and reflux condenser was added red phosphorus (1.85 g, 60 mmol) and valerolactone (12.0 g, 120 mmol). The reaction was cooled to 0° C. with an ice bath and bromine (42.1 g, 264 mmol) was added dropwise. After the addition was complete, the dark slurry was heated with an oil bath at 50° C. for 12 hours. The reaction was cooled to ambient temperature and transferred to a fresh round bottom for distillation. The 2,5-dibromopentanoyl bromide (17.5 g, 45%) was isolated under a vacuum of 0.5 mm mercury at 83-85° C. as a clear liquid and used without any further purification.

Step B:

To solution of crude 2,5-dibromopentanoyl bromide (6.50 g, 20 mmol) and triethylamine (3.06 g, 30.2 mmol) in CH₂Cl₂ (40 mL) cooled to 0° C. was added tert-butyl-4-aminopiperidine-1-carboxylate (4.23 g, 21.1 mmol) in one portion. The reaction was allowed to stir for 4 hours and then was quenched by the addition of saturated aqueous NaHCO₃ (5 mL) and CH₂Cl₂ (10 mL). The organic layer was separated and the aqueous layer washed with additional CH₂Cl₂ (3×15 mL). The combined organic layers were dried over MgSO₄ and concentrated. The crude material was purified over silica gel (3:1 hexanes/EtOAc) to give tert-butyl 4-(2,5-dibromopentanamido)piperidine-1-carboxylate (8.00 g, 90%) as a white solid.

Step C:

To a solution of tert-butyl 4-(2,5-dibromopentanamido)piperidine-1-carboxylate (2.21 g, 5.00 mmol) dissolved in DMF (10 mL) was added 60% sodium hydride (0.220 g, 5.5 mmol). The reaction was allowed to stir for 2 hours. The reaction was concentrated under vacuum and then diluted with water and EtOAc. The organic layer was separated and the aqueous layer washed with EtOAc (2×20 mL). The combined organic layers were dried over MgSO₄ and concentrated. The residue was purified over silica gel (2:1 hexanes/EtOAc) to give tert-butyl 3-bromo-2-oxo-1,4′-bipiperidine-1′carboxylate (1.35 g, 75%) as a white solid.

Step D:

To a solution of tert-butyl 3-bromo-2-oxo-1,4′-bipiperidine-1′carboxylate (723 mg, 2.00 mmol) in DMF (5 mL) was added K₂CO₃ (553 mg, 4.00 mmol) and 4-bromo-2,5-difluorophenol (460 mg, 2.20 mmol). The reaction was heated at 60° C. overnight. The reaction was cooled and diluted with water and EtOAc. The organic layer was separated and the aqueous layer was washed with EtOAc (2×10 mL). The combined organic layers were dried over MgSO₄ and concentrated. The crude residue was purified over silica gel (2:1 hexanes/EtOAc) to give tert-butyl 3-(4-bromo-2,5-difluorophenoxy)-2-oxo-1,4′-bipiperidine-1′-carboxylate (710 mg, 73%) as a white solid.

Step E:

To a solution of tert-butyl 3-(4-bromo-2,5-difluorophenoxy)-2-oxo-1,4′-bipiperidine-1′-carboxylate (710 mg, 1.45 mmol) in DMSO (4 mL) was added Cu(I) triflate benzene complex (73 mg, 0.145 mmol), sodium methanesulfinate (222 mg, 2.18 mmol) and trans-cyclohexane-1,2-diamine (66.3 mg, 0.580 mmol). The reaction was heated at 110° C. overnight. The reaction was cooled to ambient temperature and diluted with water (5 mL) and EtOAc (5 mL). The organic layer was separated and the aqueous layer was washed with EtOAc (2×10 mL). The combined organic layers were dried over MgSO₄ and concentrated. The crude residue was purified over silica gel (2:1 hexanes/EtOAc) to give a clear oil. The oil was dissolved in CH₂Cl₂ (10 mL) and 5 mL HCl (2 N in ether) was added. The reaction was allowed to stir overnight and then concentrated under vacuum to give 3-(2,5-difluoro-4-(methylsulfonyl)phenoxy)-[1,4′-bipiperidin]-2-one HCl salt (238 mg, 89%) was an off white solid.

Step F:

A suspension of N-(methylsulfonyloxy)isobutyrimidoyl chloride (42 mg, 0.21 mmol) and sodium isothiocyanate (19 mg, 0.23 mmol) in EtOAc (4 mL) was heated at 60° C. for 1 hour. 3-(2,5-Difluoro-4-(methylsulfonyl)phenoxy)-[1,4′-bipiperidin]-2-one (80 mg, 0.18 mmol) was added and the reaction was stirred at 60° C. overnight. The reaction was cooled to ambient temperature and diluted with water (3 mL) and EtOAc (5 mL). The organic layer was separated and the aqueous washed with EtOAc (2×10 mL). The combined organic layers were dried over MgSO₄ and concentrated. The crude residue was purified by reverse phase HPLC (5 to 95% acetonitrile in water with 0.1% TFA). The fractions were concentrated to remove CH₃CN and the pH adjusted to neutral by the addition of NaHCO₃. The aqueous layer was extracted with EtOAc (3×10 mL). The combined organics were dried over MgSO₄ and concentrated to give 3-(2,5-difluoro-4-(methylsulfonyl)phenoxy)-1′-(3-isopropyl-1,2,4-thiadiazol-5-yl)-[1,4′-bipiperidin]-2-one (15 mg, 14%) as an off white solid. Mass spectrum (apci) m/z=515.2 (M+H).

### Example 86

(S)-3-(2-Fluoro-4-(methylsulfonyl)phenoxy)-1-(1-(3-(trifluoromethyl)-1,2,4-thiadiazol-5-yl)piperidin-4-yl)pyrrolidin-2-one

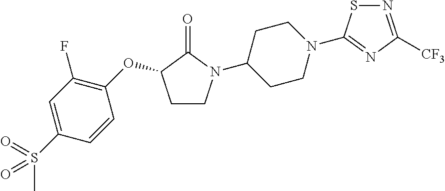 [SMILES: CS(=O)(=O)c1ccc(O[C@H]2CCN(C3CCN(c4nc(C(F)(F)F)ns4)CC3)C2=O)c(F)c1]

To a solution of (S)-3-(2-fluoro-4-(methylsulfonyl)phenoxy)-1-(piperidin-4-yl)pyrrolidin-2-one (Preparation II; 0.10 g, 0.28 mmol) in DMF was added N-ethyl-N-isopropylpropan-2-amine (0.11 g, 0.84 mmol) and 5-chloro-3-(trifluoromethyl)-1,2,4-thiadiazole (0.21 g, 1.1 mmol) and the reaction was stirred at ambient temperature for 3 hours. The reaction was diluted with EtOAc and washed with water and brine, dried over MgSO₄, filtered and concentrated in vacuo. The residue was chromatographed using 15% EtOAc in dichloromethane as eluent to yield (S)-3-(2-fluoro-4-(methylsulfonyl)phenoxy)-1-(1-(3-(trifluoromethyl)-1,2,4-thiadiazol-5-yl)piperidin-4-yl)pyrrolidin-2-one (0.027 g, 0.052 mmol, 18% yield). ¹H NMR (CDCl₃, 400 MHz) δ 1.80-1.90 (m, 4H), 2.30-2.38 (m, 1H), 2.58-2.68 (m, 1H), 3.03 (m, 3H), 3.30-3.41 (m, 3H), 3.50-3.57 (m, 1H), 4.0-4.18 (m, 2H), 4.23-4.32 (m, 1H), 5.14 (t, 1H), 7.51 (t, 1H), 7.66-7.75 (m, 2H).

The following compounds were also prepared according to the procedure for Example 86.

| | | | Mass |
|---|---|---|---|
| Ex. # | Structure | Name | spectrum |
| 87 | 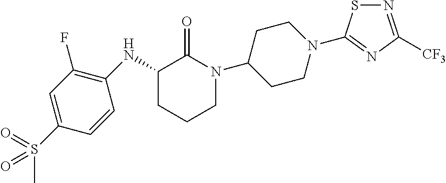 [SMILES: CS(=O)(=O)c1ccc(N[C@H]2CCCN(C3CCN(c4nc(C(F)(F)F)ns4)CC3)C2=O)c(F)c1] | (S)-3-(2-fluoro-4- (methylsulfonyl)phenylamino)- 1′-(3-(trifluoromethyl)- 1,2,4-thiadiazol-5-yl)-[1,4′- bipiperidin]-2-one | (apci) m/z = 521.9 (M + H) |
| 88 | 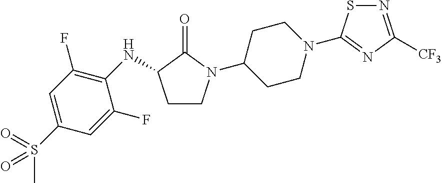 [SMILES: CS(=O)(=O)c1cc(F)c(N[C@H]2CCN(C3CCN(c4nc(C(F)(F)F)ns4)CC3)C2=O)c(F)c1] | (S)-3-(2,6-difluoro-4- (methylsulfonyl) phenylamino)-1-(1-(3- (trifluoromethyl)-1,2,4- thiadiazol-5-yl)piperidin-4- yl)pyrrolidin-2-one | (apci) m/z = 526.0 (M + H) |
| 89 | 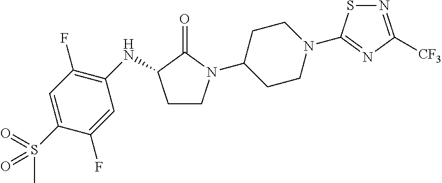 [SMILES: CS(=O)(=O)c1cc(F)c(N[C@H]2CCN(C3CCN(c4nc(C(F)(F)F)ns4)CC3)C2=O)cc1F] | (S)-3-(2,5-difluoro-4- (methylsulfonyl) phenylamino)-1-(1-(3- (trifluoromethyl)-1,2,4- thiadiazol-5-yl)piperidin-4- yl)pyrrolidin-2-one | (apci) m/z = 526.0 (M + H) |
| 90 | 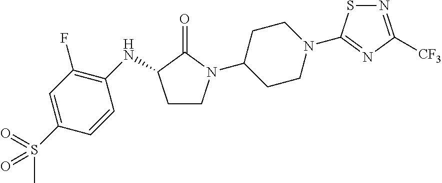 [SMILES: CS(=O)(=O)c1ccc(N[C@H]2CCN(C3CCN(c4nc(C(F)(F)F)ns4)CC3)C2=O)c(F)c1] | (S)-3-(2-fluoro-4- (methylsulfonyl)phenylamino)- 1-(1-(3-(trifluoromethyl)- 1,2,4-thiadiazol-5- yl)piperidin-4-yl)pyrrolidin- 2-one | (apci) m/z = 508.0 (M + H) |
| 91 | 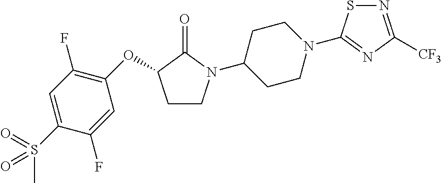 [SMILES: CS(=O)(=O)c1cc(F)c(O[C@H]2CCN(C3CCN(c4nc(C(F)(F)F)ns4)CC3)C2=O)cc1F] | (S)-3-(2,5-difluoro-4- (methylsulfonyl)phenoxy)- 1-(1-(3-(trifluoromethyl)- 1,2,4-thiadiazol-5- yl)piperidin-4-yl)pyrrolidin- 2-one | (apci) m/z = 527.1 (M + H) |

### Example 92

### 1-(1-(3-isopropyl-1,2,4-thiadiazol-5-yl)piperidin-4-yl)-3-(4-(methylsulfonyl)benzyl)pyrrolidin-2-one

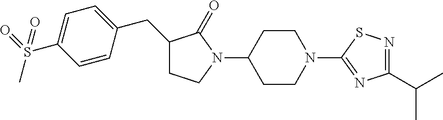 [SMILES: CC(C)c1nsc(N2CCC(N3CCC(Cc4ccc(S(C)(=O)=O)cc4)C3=O)CC2)n1]

Step A:

1-(3-Isopropyl-1,2,4-thiadiazol-5-yl)piperidin-4-amine hydrochloride (500 mg, 1.90 mmol) was suspended in THF (20 mL). Triethylamine (663 μL, 4.76 mmol) and 4-bromobutanoyl chloride (265 μL, 2.28 mmol) were added and stirred at ambient temperature for 30 minutes. The reaction was partitioned between water and EtOAc, dried over MgSO₄, filtered, and concentrated to afford 4-bromo-N-(1-(3-isopropyl-1,2,4-thiadiazol-5-yl)piperidin-4-yl)butanamide (711 mg, 1.89 mmol, 99% yield) as a white solid.

Step B:

4-Bromo-N-(1-(3-isopropyl-1,2,4-thiadiazol-5-yl)piperidin-4-yl)butanamide (711 mg, 1.89 mmol) was dissolved in DMF (10 mL). Sodium hydride (75.8 mg, 1.89 mmol) was added and the reaction was stirred at ambient temperature for 2 hours. The reaction was partitioned between EtOAc and water. The water layer was extracted with EtOAc (twice) and the combined organic layer was washed with brine, dried over MgSO₄, filtered, and concentrated to afford 1-(1-(3-isopropyl-1,2,4-thiadiazol-5-yl)piperidin-4-yl)pyrrolidin-2-one (354 mg, 1.20 mmol, 63% yield).

Step C:

1-(1-(3-Isopropyl-1,2,4-thiadiazol-5-yl)piperidin-4-yl)pyrrolidin-2-one (100 mg, 0.34 mmol) was dissolved in THF (3 mL) and cooled to −78° C. Lithium diisopropylamide (226 μL, 0.340 mmol) was added and the reaction was stirred for 5 minutes. 1-(Chloromethyl)-4-(methylsulfonyl)benzene (83.4 mg, 0.408 mmol) was added and the reaction was allowed to warm to ambient temperature for 1 hour. The reaction was partitioned between water and EtOAc and the organic layer was dried over MgSO₄, filtered, and concentrated. The crude was purified over silica gel (eluting with 2% methanol in EtOAc) to afford 1-(1-(3-isopropyl-1,2,4-thiadiazol-5-yl)piperidin-4-yl)-3-(4-(methylsulfonyl)benzyl)pyrrolidin-2-one (34 mg, 0.074 mmol, 21% yield) as a white solid. Mass spectrum (apci) m/z=463.3 (M+H).

### Example 93

### (S)-3-(2,5-difluoro-4-(methylsulfonyl)phenoxy)-1′-(3-(trifluoromethyl)-1,2,4-thiadiazol-5-yl)-[1,4′-bipiperidin]-2-one

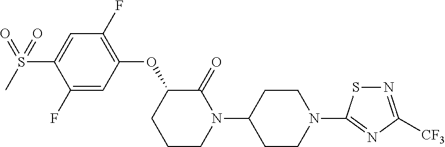 [SMILES: CS(=O)(=O)c1cc(F)c(O[C@H]2CCCN(C3CCN(c4nc(C(F)(F)F)ns4)CC3)C2=O)cc1F]

Step A:

To a stirred solution of (methoxymethyl)triphenylphosphonium chloride (1.19 g, 3.48 mmol) in anhydrous ether (50 mL) at −10° C. under nitrogen was added phenyllithium (1.93 mL, 3.48 mmol; 1.8 M solution in diethyl ether) over 1 minute using a syringe. The mixture was stirred at 0° C. for 30 minutes and then cooled to −78° C. A solution of (R)-2-(2,2-dimethyl-5-oxo-1,3-dioxolan-4-yl)acetaldehyde (Preparation EE, Step B; 0.500 g, 3.16 mmol) in ether/THF 1:1 (50 mL) was introduced via addition funnel, and the reaction mixture was stirred at −78° C. for 1 hour and then warmed to ambient temperature and stirred for 4 hours. The crude material was filtered and the residue was purified over silica gel (5-50% EtOAc in hexanes) to afford (R)-5-(3-methoxyallyl)-2,2-dimethyl-1,3-dioxolan-4-one (0.355 g, 1.90 mmol, 60% yield) as a clear, colorless oil (mixture of (E)- and (Z)-isomers).

Step B:

A solution of (R)-5-(3-methoxyallyl)-2,2-dimethyl-1,3-dioxolan-4-one (600 mg, 3.22 mmol) in acetone (32.2 mL, 3.22 mmol) and H₂SO₄ (1 drop) at ambient temperature was stirred for 70 minutes. Saturated aqueous NaHCO₃ (4-5 drops) was added and the mixture was concentrated in vacuo at ambient temperature. The residue was diluted with ether, washed with water, dried (Na₂SO₄), filtered and concentrated in vacuo to afford (R)-3-(2,2-dimethyl-5-oxo-1,3-dioxolan-4-yl)propanal (407 mg, 0.938 mmol, 58% yield) as a yellow oil (2:1 mixture of aldehyde to dimethyl acetal), which was used in the next step without purification.

Step C:

To a stirred solution of crude (R)-3-(2,2-dimethyl-5-oxo-1,3-dioxolan-4-yl)propanal (2.0 g, 8.71 mmol) in THF (120 mL) at 0° C. was added tert-butyl 4-aminopiperidine-1-carboxylate (1.92 g, 9.58 mmol). Sodium triacetoxyborohydride (2.77 g, 13.1 mmol) was added portionwise such that the internal temperature did not exceed 5° C. The mixture was stirred overnight while warming to ambient temperature. The reaction mixture was diluted with EtOAc and washed with brine. The aqueous layer was extracted twice with EtOAc, and the combined organic extracts were washed with brine, dried over magnesium sulfate, filtered and concentrated in vacuo. The residue was purified by reverse phase chromatography on C18 (0-60% ACN in water), to afford (R)-tert-butyl 3-hydroxy-2-oxo-[1,4′-bipiperidine]-1′-carboxylate as a light, white solid (1.55 g, 4.94 mmol, 57% yield). Mass spectrum (apci) m/z=199.1 (M+H-Boc).

Step D:

To a stirred solution of (R)-tert-butyl 3-hydroxy-2-oxo-1,4′-bipiperidine-1′-carboxylate (151 mg, 0.506 mmol) in THF (10 mL) at 8° C. was added N-ethyl-N-isopropylpropan-2-amine (0.176 μL, 1.01 mmol) in one portion. Methanesulfonyl chloride (47.3 μL, 0.607 mmol) was added such that the internal temperature did not exceed 5° C. After 45 minutes additional methanesulfonyl chloride (22 μL, 0.31 mmol) was added and stirring was continued for 15 minutes. To the reaction mixture was added 25 mL of EtOAc, followed by aqueous saturated NaHCO₃ (35 mL) via syringe such that the internal temperature did not exceed 5° C. The mixture was extracted with EtOAc, washed with, brine, dried (MgSO₄), filtered and concentrated in vacuo. The residue was purified over silica gel (50-100% EtOAc in hexanes) to give (R)-tert-butyl 3-(methylsulfonyloxy)-2-oxo-1,4′-bipiperidine-1′-carboxylate (105 mg, 0.273 mmol, 54% yield). ¹H NMR CDCl₃ δ 4.90 (m, 1H), 4.45 (m, 1H), 4.13 (m, 2H), 3.18 (s, 3H), 3.11 (m, 2H), 2.69 (m, 2H), 2.15 (m, 1H), 1.99 (m, 1H), 1.75 (m, 4H), 1.36 (s, 9H).

Step E:

To a stirred mixture of (R)-tert-butyl 3-(methylsulfonyloxy)-2-oxo-1,4′-bipiperidine-1′-carboxylate (1.10 g, 2.92 mmol) and potassium carbonate (485 mg, 3.51 mmol, 300 mesh, powdered) in THF (75 mL) was added 4-bromo-2,5-difluorophenol (733 mg, 3.51 mmol) and the reaction mixture was heated to reflux for 18 hours under nitrogen. The mixture was concentrated in vacuo and purified over silica gel (1:1 hexane/EtOAc) to afford (S)-tert-butyl 3-(4-bromo-2,5-difluorophenoxy)-2-oxo-1,4′-bipiperidine-1′-carboxylate obtained as a white solid (987 mg, 1.96 mmol, 67% yield). Mass spectrum (apci) m/z=389 (M+H-Boc). Chiral HPLC-analysis indicated this material was about 81% ee.

Normal Phase Chiral Method Conditions: Column: CHIRALPAK ADH (4.6×150 mm; 5 μm, Part #19324); UV: 222 nm; Sample preparation: 0.5 mg/mL methanol; Injection volume: 10 μL; Approximate retention times: (R)-enantiomer: 9.2 minutes; (S)-enantiomer: 9.8 minutes.

### Gradient:

| Time | Flow | Mobile phase A: | Mobile phase B: |
|---|---|---|---|
| (mins.) | (mL/min) | hexanes (%) | ethanol (200 proof) (%) |
| 0 | 0.8 | 90 | 10 |
| 1 | 0.8 | 90 | 10 |
| 20 | 0.8 | 5 | 95 |
| 30 | 0.8 | 5 | 95 |

Step F:

A suspension of (5)-tert-butyl 3-(4-bromo-2,5-difluorophenoxy)-2-oxo-1,4′-bipiperidine-1′-carboxylate (700 mg, 1.39 mmol), and sodium methanesulfinate (219 mg, 2.08 mmol) in DMSO (5.55 mL), was deoxygenated and purged with nitrogen. Cu(I) triflate benzene complex (77.6 mg, 0.139 mmol) and (1S,2S)-cyclohexane-1,2-diamine (63.4 mg, 0.555 mmol) were introduced and the heterogeneous mixture was sealed and heated to 110° C. in an oil bath and stirred for 18 hours. The mixture was cooled to ambient temperature, diluted with EtOAc (75 mL), washed with water (30 mL) and brine (three 50 mL washes), dried (Na₂SO₄), filtered and concentrated in vacuo. The residue was purified over silica gel (EtOAc) to afford (S)-tert-butyl 3-(2,5-difluoro-4-(methylsulfonyl)phenoxy)-2-oxo-1,4′-bipiperidine-1′-carboxylate (305 mg, 0.606 mmol, 44% yield) as a light yellow oil that solidified. Mass spectrum (apci) m/z=389.1 (M+H-Boc).

Step G:

To a stirred solution of (S)-tert-butyl 3-(2,5-difluoro-4-(methylsulfonyl)phenoxy)-2-oxo-1,4′-bipiperidine-1′-carboxylate (370 mg, 0.757 mmol) in methanol (5 mL) was added 5 M HCl in IPA (1.51 mL, 7.57 mmol) and the mixture was stirred at ambient temperature for 6 hours and concentrated in vacuo. The residue was stirred in 1M NaOH (20 mL) and DCM (25 mL). The organic layers were combined, dried (Na₂SO₄), filtered and concentrated in vacuo to give (S)-3-(2,5-difluoro-4-(methylsulfonyl)phenoxy)-[1,4′-bipiperidin]-2-one as light brown foam (282 mg, 0.726 mmol, 96% yield). Mass spectrum (apci) m/z=389.1 (M+H).

Step H:

To a solution of (S)-3-(2,5-difluoro-4-(methylsulfonyl)phenoxy)-[1,4′-bipiperidin]-2-one (20 mg, 0.051 mmol) in DMF (2 mL) was added 5-chloro-3-(trifluoromethyl)-1,2,4-thiadiazole (10 mg, 0.051 mmol) and N-ethyl-N-isopropylpropan-2-amine (20 mg, 0.15 mmol) and the reaction was stirred at ambient temperature for 2 hours. The reaction was purified by SP4 eluting 0-75% water/ACN over 30 minutes to give (S)-3-(2,5-difluoro-4-(methylsulfonyl)phenoxy)-1′-(3-(trifluoromethyl)-1,2,4-thiadiazol-5-yl)-[1,4′-bipiperidin]-2-one (19 mg, 68%,). Mass spectrum (apci) m/z=541.1 (M+H).

### Example 94

### (S)-1-(1-(3-isopropyl-1,2,4-thiadiazol-5-yl)piperidin-4-yl)-3-((6-(methylsulfonyl)pyridin-3-yl)oxy)pyrrolidin-2-one

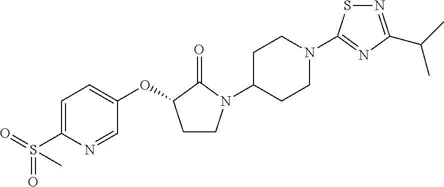 [SMILES: CC(C)c1nsc(N2CCC(N3CC[C@H](Oc4ccc(S(C)(=O)=O)nc4)C3=O)CC2)n1]

Step A:

To a suspension of (R)-3-hydroxydihydrofuran-2(3H)-one (500 mg, 4.9 mmol) in toluene (15 mL) was added triphenylphosphine (1.54 g, 5.88 mmol) and 6-bromopyridin-3-ol (1.02 g, 5.88 mmol). The solution was cooled to 0° C. and degassed with nitrogen bubble for 10 minutes. Di-tert-butyl diazene-1,2-dicarboxylate was dissolved in toluene (5 mL) and added over a 5 minute period. The reaction was allowed to stir for 12 hours with warming to ambient temperature. The reaction was concentrated in vacuo and the resulting material purified by silica gel chromatography eluting 1:1 hexanes/EtOAc to yield (S)-3-(6-bromopyridin-3-yloxy)dihydrofuran-2(3H)-one (1.0 g, 79%) as an off white solid.

Step B:

To a solution of tert-butyl 4-aminopiperidine-1-carboxylate (0.93 g, 4.65 mmol) in DCM (15 mL) was added dropwise 2M trimethylaluminum (2.8 mL, 5.7 mmol) in toluene. The resulting mixture was stirred for 15 minutes, and (S)-3-(6-bromopyridin-3-yloxy)dihydrofuran-2(3H)-one (1.0 g, 3.87 mmol) in DCM (10 mL) was added slowly over 5 minutes and the reaction stirred at ambient temperature for 2 hours. The reaction was slowly quenched by the addition of 5% tartaric acid (5 mL), saturated NaHCO₃ (5 mL) and DCM (10 mL). The organic layer was separated, dried over MgSO₄, filtered and concentrated in vacuo to provide (S)-tert-butyl 4-(2-(6-bromopyridin-3-yloxy)-4-hydroxybutanamido)piperidine-1-carboxylate. The crude material was taken on in the next step without further purification.

Step C:

A solution of (5)-tert-butyl 4-(2-(6-bromopyridin-3-yloxy)-4-hydroxybutanamido)piperidine-1-carboxylate (1.30 g, 2.84 mmol) and tributylphosphine (689 mg, 3.40 mmol) in toluene (15 mL) was degassed with nitrogen bubble for 10 minutes and then cooled to 0° C. Di-tert-butyl diazene-1,2-dicarboxylate (784 mg, 3.4 mmol) dissolved in toluene (5 mL) and was added over a 5 minute period. The reaction was allowed to warm to ambient temperature over 12 hours. The reaction was concentrated in vacuo and purified by silica gel chromatography eluting with 1:1 hexanes/EtOAc to yield (S)-tert-butyl-4-(3-(6-bromopyridin-3-yloxy)-2-oxopyrrolidin-1-yl)piperidine-1-carboxylate (934 mg, 75%) as an off-white solid.

Step D:

A solution of (S)-tert-butyl-4-(3-(6-bromopyridin-3-yloxy)-2-oxopyrrolidin-1-yl)piperidine-1-carboxylate (315 mg, 0.714 mmol) in degassed DMSO (5 mL) was added sodium ethanesulfinate (117 mg, 1.15 mmol), trans-cyclohexane-1,2-diamine (33 mg, 0.286 mmol) and Cu(I) triflate benzene complex (54 mg, 0.107 mmol). The reaction was heated to 110° C. for 12 hours, then cooled to ambient temperature and partitioned between water (5 mL) and EtOAc (10 mL). The organic layer was separated and the aqueous layer was extracted with EtOAc. The combined organic layers were dried over MgSO₄, filtered and concentrated in vacuo. The resulting material was purified by silica gel chromatography eluting with 1:1 hexanes/EtOAc to yield (S)-tert-butyl-4-(3-(6-(methylsulfonyl)pyridine-3-yloxy)-2-oxopyrrolidin-1-yl)piperidine-1-carboxylate (315 mg, 94%) as a white solid.

Step E:

To a solution of (S)-tert-butyl-4-(3-(6-(methylsulfonyl)pyridine-3-yloxy)-2-oxopyrrolidin-1-yl)piperidine-1-carboxylate (259 mg, 0.59 mmol) in DCM (10 mL) was added excess 4N HCl in dioxane (3 mL). The reaction was stirred for 4 hours, then concentrated in vacuo to yield (S)-3-(6-(methylsulfonyl)pyridin-3-yloxy)-1-(piperidin-4-yl)pyrrolidin-2-one hydrochloride salt (210 mg, 94%) as an off white solid, which was used in the next step without further purification.

Step F:

To a solution of N-(methylsulfonyloxy)isobutyrimidoyl chloride (62 mg, 0.32 mmol) in EtOAc/CH₃CN (3:1 mL) was added NaNCS (25 mg, 0.32 mmol) and pyridine (192 mg, 2.4 mmol). The reaction was heated to 60° C. for 45 minutes. (S)-3-(6-(methylsulfonyl)pyridin-3-yloxy)-1-(piperidin-4-yl)pyrrolidin-2-one hydrochloride salt (100 mg, 0.26 mmol) was added and the reaction allowed to stir at 60° C. overnight. The reaction was quenched by the addition of water (5 mL) and EtOAc (5 mL). The organic layer was separated and the aqueous layer was extracted with EtOAc (2×5 mL). The combined organic layers were dried over MgSO₄, filtered and concentrated in vacuo. The resulting residue was purified by silica gel chromatography, eluting with Hexanes/EtOAc (1:1)—100% EtOAc to give (S)-1-(1-(3-isopropyl-1,2,4-thiadiazol-5-yl)piperidin-4-yl)-3-((6-(methylsulfonyl)pyridin-3-yl)oxy)pyrrolidin-2-one (52 mg, 46%) Mass spectrum (apci) m/z=466.2 (M+H).

### Example 95

### (S)-1-(1-(2-(tert-butyl)2H-tetrazol-5-yl)piperidin-4-yl)-3-(2-fluoro-4-(methylsulfonyl)phenoxy)pyrrolidin-2-one

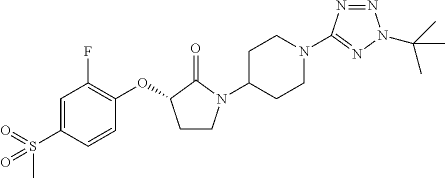 [SMILES: CC(C)(C)n1nnc(N2CCC(N3CC[C@H](Oc4ccc(S(C)(=O)=O)cc4F)C3=O)CC2)n1]

Step A:

To a solution of cyanic bromide (0.25 g, 2.4 mmol) in acetonitrile (40 mL) was added potassium carbonate (0.39 g, 2.8 mmol) and (S)-3-(2-difluoro-4-(methylsulfonyl)phenoxy)-1-(piperidin-4-yl)pyrrolidin-2-one (0.50 g, 1.34 mmol) and the reaction stirred for 1.5 hours at ambient temperature. The reaction was quenched with 1 N NaOH and extracted with EtOAc. The organic layer was separated and washed with 1 N NaOH and brine, dried over MgSO₄ and filtered. The filtrate was concentrated under vacuum to give (S)-4-(3-(2-difluoro-4-(methylsulfonyl)phenoxy)-2-oxopyrrolidin-1-yl)piperidine-1-carbonitrile (0.48 g, 1.2 mmol, 89% yield).

Step B:

(S)-4-(3-(2-Fluoro-4-(methylsulfonyl)phenoxy)-2-oxopyrrolidin-1-yl)piperidine-1-carbonitrile (0.300 g, 0.787 mmol) was dissolved in DMF (4 mL). NH₄Cl (0.0421 g, 0.787 mmol) and NaN₃ (0.205 g, 3.15 mmol) were added. The reaction was heated at 100° C. for 2 hours. The reaction was quenched with water and concentrated in vacuo to obtain (S)-1-(1-(2H-tetrazol-5-yl)piperidin-4-yl)-3-(2-fluoro-4-(methylsulfonyl)phenoxy)pyrrolidin-2-one (0.6 g, 1.41 mmol, 180% yield) as a white solid. The crude material was carried forward into the next reaction.

Step C:

To a solution of (S)-1-(1-(2H-tetrazol-5-yl)piperidin-4-yl)-3-(2-fluoro-4-(methylsulfonyl)phenoxy)pyrrolidin-2-one (0.200 g, 0.471 mmol) and 2,2,2-trifluoroacetic acid (1.61 g, 14.1 mmol) in DCM (50 mL) was added concentrated sulfuric acid (15 μL) and 2-methylpropan-2-ol (0.524 g, 7.07 mmol). The mixture was allowed to stir at ambient temperature for 1 hour and at 45° C. for 5 hours. The solution was cooled, diluted with saturated NaHCO₃ and extracted with DCM. The combined organic layers were dried over MgSO₄, filtered and concentrated. Purification of the crude material by flash chromatography (EtOAc) gave (S)-1-(1-(2-tert-butyl-2H-tetrazol-5-yl)piperidin-4-yl)-3-(2-fluoro-4-(methylsulfonyl)phenoxy)pyrrolidin-2-one (0.111 g, 0.231 mmol, 49.0% yield) Mass spectrum (apci) m/z=481.1 (M+H).

### Example 96

### (S)-3-(2,5-difluoro-4-(methylsulfonyl)phenoxy)-1-(1-(5-(trifluoromethyl)-1,3,4-thiadiazol-2-yl)piperidin-4-yl)pyrrolidin-2-one

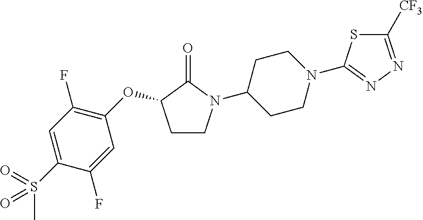 [SMILES: CS(=O)(=O)c1cc(F)c(O[C@H]2CCN(C3CCN(c4nnc(C(F)(F)F)s4)CC3)C2=O)cc1F]

To a solution of (S)-3-(2,5-difluoro-4-(methylsulfonyl)phenoxy)-1-(piperidin-4-yl)pyrrolidin-2-one (Preparation JJ; 0.30 g, 0.80 mmol) in DMF (2 mL) was added potassium carbonate (0.133 g, 0.96 mmol) and 2-chloro-5-(trifluoromethyl)-1,3,4-thiadiazole (0.181 g, 0.962 mmol). The reaction was stirred at 75° C. for 3 hours. The reaction was diluted with EtOAc and washed with water and brine, dried over MgSO₄, filtered and concentrated in vacuo. The residue purified by flash chromatography to yield (S)-3-(2,5-difluoro-4-(methylsulfonyl)phenoxy)-1-(1-(5-(trifluoromethyl)-1,3,4-thiadiazol-2-yl)piperidin-4-yl)pyrrolidin-2-one (0.111 g, 0.211 mmol, 26.3% yield). Mass spectrum (apci) m/z=527.0 (M+H).

### Example 97

### (S)-3-(2,5-difluoro-4-(methylsulfonyl)phenoxy)-1-(1-(2-(trifluoromethyl)oxazol-5-yl)piperidin-4-yl)pyrrolidin-2-one

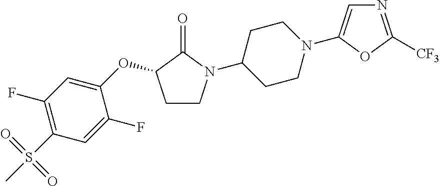 [SMILES: CS(=O)(=O)c1cc(F)c(O[C@H]2CCN(C3CCN(c4cnc(C(F)(F)F)o4)CC3)C2=O)cc1F]

Step A:

(S)-3-(2,5-difluoro-4-(methylsulfonyl)phenoxy)-1-(piperidin-4-yl)pyrrolidin-2-one (Preparation JJ; 4.00 g, 10.7 mmol) in NMP (10 mL) was charged with 2-(tert-butoxycarbonylamino)acetic acid (2.25 g, 12.8 mmol) and HOBT-H₂O (1.96 g, 12.8 mmol). EDCI (3.07 g, 16.0 mmol) was added and the reaction stirred at ambient temperature overnight. The reaction was diluted with ethyl acetate (50 mL), washed with water and saturated sodium bicarbonate (2×50 mL). The combined organic layers were dried over MgSO₄, filtered and concentrated in vacuo to give (S)-tert-butyl 2-(4-(3-(2,5-difluoro-4-(methylsulfonyl)phenoxy)-2-oxopyrrolidin-1-yl)piperidin-1-yl)-2-oxoethylcarbamate (3.07 g, 5.78 mmol).

Step B:

(S)-tert-butyl 2-(4-(3-(2,5-difluoro-4-(methylsulfonyl)phenoxy)-2-oxopyrrolidin-1-yl)piperidin-1-yl)-2-oxoethylcarbamate (3.07 g, 5.78 mmol) was dissolved in 4M HCl in dioxane and the mixture was stirred for 1 hour. The mixture was charged with ether and the solid was filtered to afford (S)-1-(1-(2-aminoacetyl)piperidin-4-yl)-3-(2,5-difluoro-4-(methylsulfonyl)phenoxy)pyrrolidin-2-one hydrochloride. The crude material was used directly in the next step without purification.

Step C:

(S)-1-(1-(2-aminoacetyl)piperidin-4-yl)-3-(2,5-difluoro-4-(methylsulfonyl)phenoxy)pyrrolidin-2-one hydrochloride (1.5 g, 3.21 mmol) in MeCN (15 mL) was treated with 2,2,2-trifluoroacetic anhydride (0.875 g, 4.17 mmol). The reaction stirred at ambient temperature for 1 hour. The solvent was removed and the residue was partitioned between EtOAc and saturated NaHCO₃. The aqueous phase was extracted with EtOAc and the combined organics were dried and concentrated to give (S)—N-(2-(4-(3-(2,5-difluoro-4-(methylsulfonyl)phenoxy)-2-oxopyrrolidin-1-yl)piperidin-1-yl)-2-oxoethyl)-2,2,2-trifluoroacetamide (0.629 g, 1.19 mmol, 37.2% yield).

Step D:

To a solution of PPh₃ (0.200 g, 0.763 mmol) in dry degassed DCM (2 mL) was added bromine (0.0391 ml, 0.763 mmol). The mixture stirred for 40 minutes at ambient temperature. Triethylamine (0.266 mL, 1.91 mmol) and (S)—N-(2-(4-(3-(2,5-difluoro-4-(methylsulfonyl)phenoxy)-2-oxopyrrolidin-1-yl)piperidin-1-yl)-2-oxoethyl)-2,2,2-trifluoroacetamide (0.334 g, 0.633 mmol) were then added and the mixture was heated at 40° C. for 1 hour. The solution was cooled to ambient temperature and stirred for 3 hours. The solution was diluted with water and extracted with EtOAc. The organic layer was separated, dried over MgSO₄, filtered and concentrated in vacuo. Flash chromatography of the crude material gave impure material which was further purified by reverse phase HPLC to give (S)-3-(2,5-difluoro-4-(methylsulfonyl)phenoxy)-1-(1-(2-(trifluoromethyl)oxazol-5-yl)piperidin-4-yl)pyrrolidin-2-one (0.0731 g, 0.143 mmol, 18.8% yield). Mass spectrum (apci) m/z=551.0 (M+MeCN).

### Example 98

### (S)-3-(2,5-difluoro-4-(methylsulfonyl)phenoxy)-1-(1-(2-(trifluoromethyl)thiazol-5-yl)piperidin-4-yl)pyrrolidin-2-one

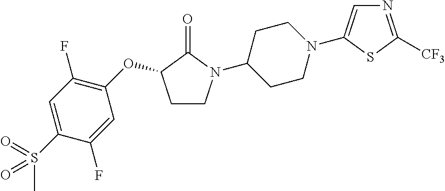 [SMILES: CS(=O)(=O)c1cc(F)c(O[C@H]2CCN(C3CCN(c4cnc(C(F)(F)F)s4)CC3)C2=O)cc1F]

(S)—N-(2-(4-(3-(2,5-difluoro-4-(methylsulfonyl)phenoxy)-2-oxopyrrolidin-1-yl)piperidin-1-yl)-2-oxoethyl)-2,2,2-trifluoroacetamide (Example 97, Steps A-C, 0.250 g, 0.474 mmol) was suspended in toluene (2 mL) and degassed with nitrogen for 5 minutes. 2,4-Bis-(4-methoxy-phenyl)-[1,3,2,4]dithiadiphosphetane 2,4-disulfide (0.192 g, 0.474 mmol) (Lawesson's reagent) was then added and the vial was capped and stirred in an oil bath at 120° C. overnight. The solution was cooled and diluted with water. The solution was extracted with EtOAc, dried over MgSO₄, filtered and concentrated in vacuo. Purification of the crude material by reverse phase HPLC gave (S)-3-(2,5-difluoro-4-(methylsulfonyl)phenoxy)-1-(1-(2-(trifluoromethyl)thiazol-5-yl)piperidin-4-yl)pyrrolidin-2-one (0.0251 g, 0.0478 mmol, 10.1% yield). Mass spectrum (apci) m/z=526.1 (M+H).

1. A compound having the general formula I 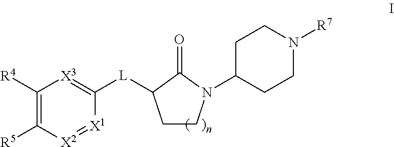
or a pharmaceutically acceptable salt or solvate thereof, wherein:
L is O, NR^{x} or CH₂;
R^{x} is H or (1-3C)alkyl;
X¹ is N or CR¹, X² is N or CR², and X³ is N or CR³, wherein only one of X¹ and X² may be N;
R¹, R², R³ and R⁴ are independently selected from H, halogen, CF₃, (1-6C)alkyl, CN and (1-6C)alkoxy;
R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, Br, tetrazolyl optionally substituted with (1-3C)alkyl, or oxadiazolyl optionally substituted with (1-3C)alkyl;
R′, R″ and R′″ are independently H or (1-4C)alkyl;
R⁷ is selected from 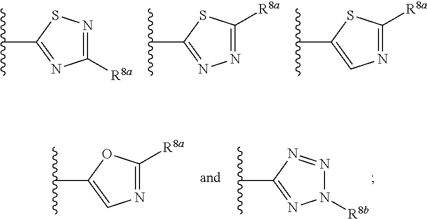
R^{8a} is selected from (1-6C)alkyl, fluoro(1-6C)alkyl, difluoro(1-6C)alkyl, trifluoro(1-6C)alkyl, (1-3C alkoxy)(1-6C)alkyl, dihydroxy(2-6C)alkyl, Br, Cyc¹, Ar¹, —OAr¹, hetCyc¹, hetAr¹ and —OhetAr¹;
R^{8b} is (1-6C)alkyl;
Cyc¹ is (3-6C)cycloalkyl optionally substituted with CF₃;
Ar¹ is phenyl optionally substituted with one or more groups independently selected from halogen, CF₃, (1-4C)alkyl and (1-4C)alkoxy;
hetCyc¹ is a 5-6 membered heterocycle having 1-2 ring heteroatoms and optionally substituted with one or more groups independently selected from (1-4C)alkyl;
hetAr¹ is a 5-6-membered heteroaryl having 1-2 ring heteroatoms and optionally substituted with one or more substituents independently selected from (1-4C)alkyl, halogen, CF₃ and (1-4C)alkoxy; and
n is 1, 2 or 3.

2. The compound of claim 1, wherein X¹ is CR¹, X² is CR², and X³ is CR³.

3. The compound of claim 2, wherein R¹, R², R³ and R⁴ are independently selected from H, F, Cl, CF₃, CN, methyl, ethyl, and propyl.

4. The compound of claim 3, wherein R¹ is H, F, Cl, CF₃, CN or Me, R² is H, F or Cl, R³ is H, and R⁴ is H, Me, F, or Cl.

5-10. (canceled)

11. The compound according to claim 1, wherein R⁵ is selected from (1-3C alkyl)sulfonyl, (3-6C cycloalkyl)sulfonyl, (cyclopropylmethyl)sulfonyl and phenylsulfonyl.

12. The compound of claim 11, wherein R⁵ is CH₃SO₂—, CH₃CH₂SO₂—, CH₃CH₂CH₂SO₂—, (CH₃)₂CHSO₂—, (cyclopropyl)SO₂—, (cyclopropylmethyl)sulfonyl or phenylsulfonyl.

13-15. (canceled)

16. The compound according to claim 1, wherein R⁷ is selected from the structures: 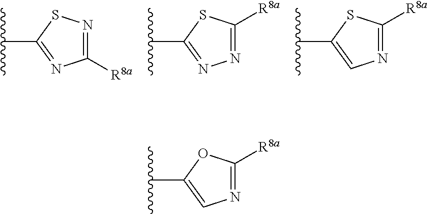
where R^{8a} is selected from (1-6C)alkyl, fluoro(1-6C)alkyl, difluoro(1-6C)alkyl, trifluoro(1-6C)alkyl, (1-3C alkoxy)(1-6C)alkyl, Cyc¹, Ar¹, —OAr¹, hetCyc¹, hetAr¹, —OhetAr¹, —CH(OH)CH₂OH, —C(CH₃)₂(OMe) and Br.

17. The compound of claim 16, wherein R^{8a} is selected from (1-6C)alkyl, fluoro(1-6C)alkyl, difluoro(1-6C)alkyl, trifluoro(1-6C)alkyl, (1-3C alkoxy)(1-6C)alkyl, Cyc¹, and -dihydroxy(2-6C)alkyl.

18. The compound of claim 17, wherein R⁸ is (1-6C)alkyl.

19-21. (canceled)

22. The compound according to claim 1, wherein L is O.

23-24. (canceled)

25. The compound according to claim 1, wherein n is 1.

26. The compound according to claim 1, wherein n is 2.

27. (canceled)

28. The compound according to claim 1, having the absolute configuration of Formula I-a:

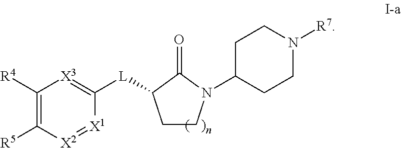

29. The compound according to claim 1, having the absolute configuration of Formula I-b:

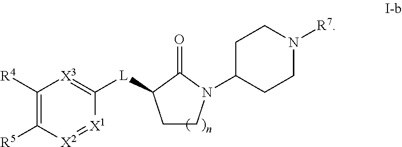

30. A pharmaceutical composition, which comprises a compound of Formula I as defined in claim 1 or a pharmaceutically acceptable salt thereof, and a pharmaceutically acceptable diluent, carrier or excipient.

31. A method of treating a disease or condition selected from type 2 diabetes, symptoms of diabetes, diabetic complications, metabolic syndrome (including hyperglycemia, impaired glucose tolerance, and insulin resistance), obesity, dyslipidemia, dyslipoproteinemia, vascular restenosis, diabetic retinopathy, hypertension, cardiovascular disease, Alzheimer's disease, schizophrenia, and multiple sclerosis in a mammal, which comprises administering to said mammal a therapeutically effective amount of a compound of Formula I as defined in claim 1 or a pharmaceutically acceptable salt thereof.

32. The method of claim 31, wherein the disease is type 2 diabetes.

33-34. (canceled)

35. A process for the preparation of a compound of claim 1, which comprises:
(a) for a compound of Formula I where L is NR^{x}, coupling a corresponding compound of Formula II 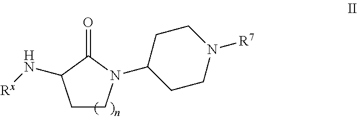
where R^{x}, R⁷ and n are as defined for Formula I, with a corresponding compound having the formula: 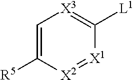
where X¹, X², X³ and R⁵ are as defined for Formula I and L¹ is a leaving group or atom, in the presence of (i) an alkali metal hydride or carbonate or (ii) a palladium catalyst and a ligand; or
(b) for a compound of Formula I where L is O, coupling a corresponding compound of Formula III 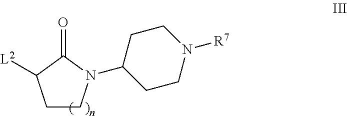
where n and R⁷ are as defined for Formula I and L² is a leaving atom, with a compound having the formula: 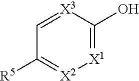
where X¹, X², X³ and R⁵ are as defined for Formula I, in the presence of a base; or
(c) for a compound of Formula I where L is CH₂, coupling a corresponding compound of Formula IV 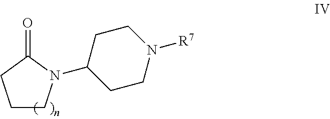
where n and R⁷ are as defined for Formula I, with a compound having the formula: 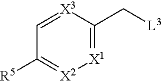
where X¹, X², X³ and R⁵ are as defined for Formula I and L³ is a leaving group of atom, in the presence of a base; or
(d) for a compound of Formula I where R⁷ is 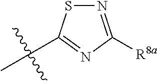
and R^{8a} is as defined for Formula I, reacting a corresponding compound of Formula V 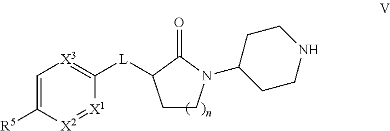
where R⁵, X¹, X², X³, L and n are as defined for Formula I, with a corresponding compound having the formula 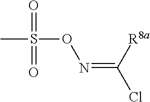
or a protected form thereof, where R^{8a} is as defined for Formula I, in the presence of sodium isothiocyanate and a base; or
(e) for a compound of Formula I where R⁷ is 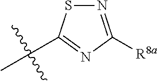
and R^{8a} is as defined for Formula I, reacting a corresponding compound of Formula V 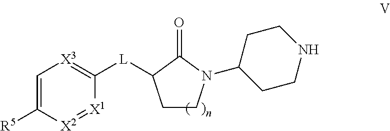
where R⁵, X¹, X², X³, L and n are as defined for Formula I, with a corresponding compound having the formula 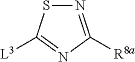
where L³ is a leaving group or atom and R^{8a} is as defined for Formula I, in the presence of a base; or
(f) for a compound of Formula I where R⁵ is (1-3C alkyl)sulfonyl, (3-6C cycloalkyl)sulfonyl, (cyclopropylmethyl)sulfonyl or phenylsulfonyl, reacting a corresponding compound having the Formula VI 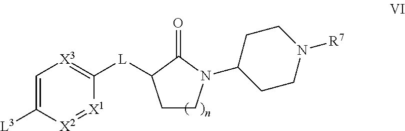
where X¹, X², X³, L, n and R⁷ are as defined for Formula I and L³ is a leaving group or atom, with a compound having the formula R^{y}SO₂Na where R^{y} is (1-3C)alkyl, (3-6C)cycloalkyl, cyclopropylmethyl or phenyl, in the presence of a base and a metal catalyst; or
(g) for a compound of Formula I where R⁵ is (1-3C alkyl)sulfonyl, (3-6C cycloalkyl)sulfonyl, (cyclopropylmethyl)sulfonyl or phenylsulfonyl, treating a corresponding compound having the Formula VII 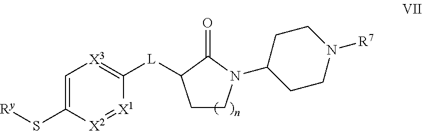
where R^{y} is (1-3C)alkyl, (3-6C)cycloalkyl, cyclopropylmethyl or phenyl, with an oxidizing agent; or
(h) for a compound of Formula I where R⁵ is CN, reacting a corresponding compound of Formula VIII 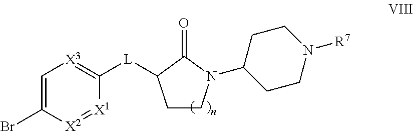
where X¹, X², X³, L, n and R⁷ are as defined for Formula I, with Cu(I)CN; or
(i) for a compound of Formula I where R⁵ is cyclopropylsulfonyl, treating a corresponding compound of Formula IX 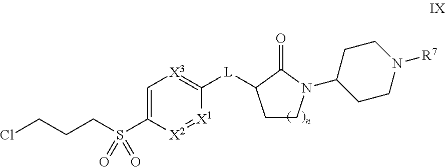
where X¹, X², X³, L, n and R⁷ are as defined for Formula I, with a base; or
(j) for a compound of Formula I where R⁵ is HOCH₂CH₂NHC(═O)— or R′R″NCH₂CH₂NR′″C(═O)— where R′, R″ and R′″ are as defined for Formula I, reacting a compound having the Formula X 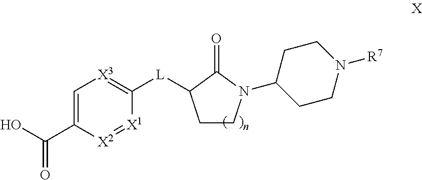
with a corresponding compound having the formula HOCH₂CH₂NH₂ or R′R″NCH₂CH₂NHR′″, respectively, in the presence of a coupling agent; or
(k) for a compound of Formula I where R⁷ is 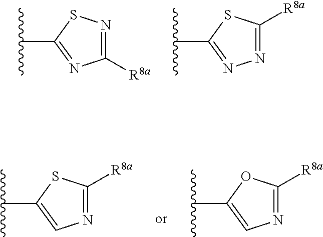
R^{8a} is hetAr¹, R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, and X¹, X², X³, L and n and are as defined for Formula I, coupling a corresponding compound of Formula XI 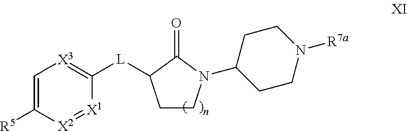
where R^{7a} is 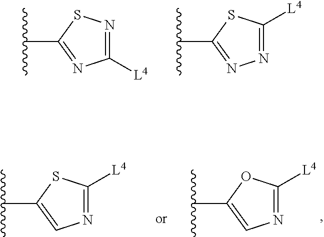
respectively, R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, and X¹, X², X³, L and n and are as defined for Formula I and L⁴ is a leaving atom, with a corresponding compound having the formula 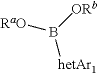
where hetAr¹ is as defined for Formula I and R^{a} and R^{b} are H or (1-6C)alkyl, or R^{a} and R^{b} together with the atoms to which they are connected form a 5-6 membered ring optionally substituted with 1-4 substituents selected from (1-3C alkyl), wherein said coupling takes place in the presence of a palladium catalyst and base and optionally in the presence of a ligand; or
(l) for a compound of Formula I where R⁷ is 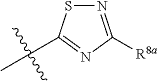
R^{8a} is —OAr¹ or —OhetAr¹, and R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, coupling a corresponding compound of Formula XI 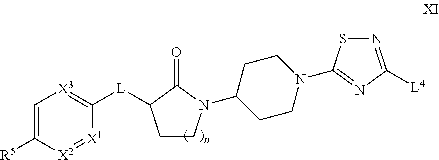
where R⁵ is (1-3C alkyl)sulfonyl, (3-6C cycloalkyl)sulfonyl, (cyclopropylmethyl)sulfonyl, phenylsulfonyl, di(1-3C alkyl)NSO₂—, (1-3C alkyl)S—, HOCH₂CH₂NHC(═O)—, R′R″NCH₂CH₂NR′″C(═O)—, CN, tetrazolyl optionally substituted with (1-3C)alkyl, or oxadiazolyl optionally substituted with (1-3C)alkyl, X¹, X², X³, L and n are as defined for Formula I and L⁴ is a leaving group or atom, with a corresponding compound having the formula HO—Ar¹ or HO-hetAr¹, respectively, in the presence of a base; or
(m) for a compound of Formula I where R⁷ is 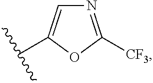 [SMILES: *c1cnc(C(F)(F)F)o1]
reacting a compound of Formula XII 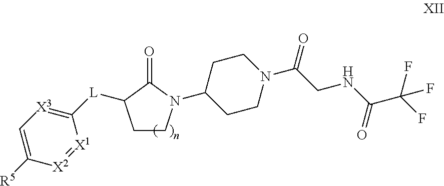
where X¹, X², X³, L, n and R⁵ are as defined for Formula I, with triphenylphosphine and bromine in the presence of a base; or
(n) for a compound of Formula I where R⁷ is 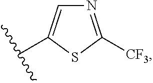 [SMILES: *c1cnc(C(F)(F)F)s1]
reacting a compound of Formula XII 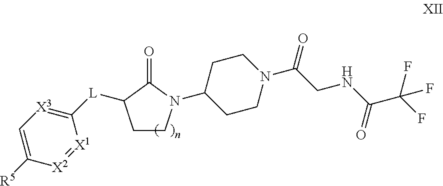
where X¹, X², X³, L, n and R⁵ are as defined for Formula I, with a thiation agent; or
(o) for a compound of Formula I where R⁷ is 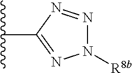
and R^{8b} is (1-6C)alkyl, reacting a corresponding compound having the formula XIII 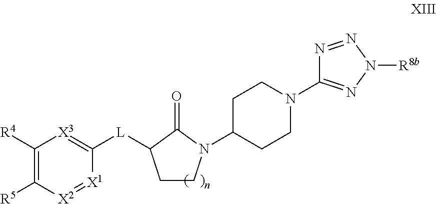
with (1-6C alkyl)OTf in the presence of an acid; and
optionally removing any protecting groups and optionally preparing a pharmaceutically acceptable salt thereof.
